(19) United States
(12) Patent Application Publication
(10) Pub. No.: US 2025/0283177 A1
(43) Pub. Date: Sep. 11, 2025

(54) BIOMARKERS AND METHODS OF USE THEREOF FOR TREATMENT OF PERIPHERAL T-CELL LYMPHOMA

(71) Applicants: BOARD OF REGENTS OF THE UNIVERSITY OF NEBRASKA, Lincoln, NE (US); THE GOV. OF THE USA AS REPRESENTED BY THE SECRETARY OF THE DEPARTMENT OF HEALTH AND HUMAN SERVICES, Rockville, MD (US)

(72) Inventors: Javeed Iqbal, Omaha, NE (US); Wing C. Chan, Pasadena, CA (US); George Wright, Rockville, MD (US); Louis M. Staudt, Bethesda, MD (US)

(21) Appl. No.: 18/837,058

(22) PCT Filed: Feb. 8, 2023
(86) PCT No.: PCT/US2023/012625

§ 371 Date: Aug. 8, 2024

Related U.S. Application Data
(60) Provisional application No. 63/307,905, filed on Feb. 8, 2022.

(51) Int. Cl. C12Q 1/6886

(52) U.S. Cl. CPC C12Q 1/6886; C12Q 2600/112; C12Q 2600/158

(57) ABSTRACT

The present disclosure is directed to methods of genetically subtypin peripheral t-cell lymphoma.

This application is a National Phase application of an International Application No. PCT/US2023/012625, filed on Feb. 8, 2023, which claims the benefit of priority from U.S. provisional patent application No. 63/307,905, filed on Feb. 8, 2022, the entire contents of each of which are incorporated herein by reference in their entireties.

All patents, patent applications, and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTEREST

This invention was made with government support under U01 CA157581 and UH3 CA206127 awarded by the National Institutes of Health (NIH). The government has certain rights in the invention.

### TECHNICAL FIELD

The presently disclosed subject matter relates to methods for diagnosis, prognosis, and/or treatment of peripheral T-cell lymphoma (PTCL). Additionally, the presently disclosed subject matter relates to a kit and reagents for diagnosis, prognosis, and/or treatment of PTCL. Further, the presently disclosed subject matter relates to methods of identification of a set of biomarkers for diagnosis, prognosis, and/or treatment of PTCL using software and analytical systems.

### Introduction

Malignancies derived from the T-cell and natural killer (NK) cell lineages comprise 10% of all non-Hodgkin lymphomas (NHLs) in Western countries and are more prevalent in Asia. Peripheral T-cell lymphoma (PTCL) is a heterogeneous cancer that constitutes up to 20% of all non-Hodgkin lymphoma (NHL). The World Health Organization (WHO) classification recognizes a number of distinctive subtypes of peripheral T-cell lymphoma (PTCL), including angioimmunoblastic T-cell lymphoma (AITL), anaplastic large cell lymphoma (ALCL), adult T-cell leukemia/lymphoma (ATLL), and entities derived mostly from NK cells including extranodal NK/T-cell lymphoma of the nasal type (ENKTL). There are additional rare PTCLs that are mostly extranodal tumors. The varied morphology and lack of definitive diagnostic markers for most of the subtypes make the diagnosis and classification of these diseases challenging and impede their investigation. With current immunophenotypic and molecular markers, about 30% to 50% of PTCL cases are not classifiable and are categorized as PTCL not otherwise specified (PTCL-NOS). Although a few recurrent genetic abnormalities have been reported in PTCL, no specific genetic abnormality is diagnostic of particular PTCL subtypes, with the exception of t(2;5)(p23;q35) in ALK(1)ALCL. Patients with PTCL generally have a poor prognosis with current standard-of-care therapy, and no improvement in the outcome of PTCL patients has been achieved in the last two decades. Therefore, accurate diagnosis of PTCL and its subtypes is needed to ensure selection of the most appropriate and effective treatment for each individual patient. In particular, the creation of a commercial assay for routine clinical application will improve the diagnosis, treatment, and outcomes of PTCL patients.

### SUMMARY OF THE INVENTION

In a first aspect, the present invention includes a method of differentiating between subtypes of Peripheral T-Cell Lymphoma (PTCL). In embodiments, the method comprises subjecting a sample from a subject to nucleic acid isolation; obtaining a gene expression profile from the sample; and identifying the subtype of PTCL based on the presence of specific genes within the gene expression profile. In certain embodiments, the PTCL subtype angioimmunoblastic T-cell lymphoma (AITL), anaplastic lymphoma kinase (ALK)-negative anaplastic large cell lymphoma (ALK−ALCL), adult T-cell leukemia/lymphoma (ATLL), extranodal natural killer/T-cell lymphoma (ENKTL), ALK-positive ALCL (ALK+ALCL), PTCL—not otherwise specified (PTCL-NOS), or indeterminate, wherein indeterminate indicates that the sample comprises features of at least two of the PTCL subtypes.

The PTCL subtype can be identified as AITL if the gene expression profile comprises any one or more of ACHE, ADRA2A, ARRDC4, COL4A4, EFNB2, FCAMR, GJA4, GNA14, IER3IP1, ISG15, MSR1, OLFM1, PAPLN, S1PR3, SOX8, TMEM206, TUBB2B, and VANGL2. The PTCL subtype can be identified as ALK−ALCL if the gene expression profile comprises any one or more of BATF3, C9orf89, CD28, DUSP2, ICMT, LGALS1, PIK3IP1, PRKCQ, TNFRSF8, TRAT1, and ZNF101. In embodiments, the PTCL subtype is identified as ENKTL if the gene expression profile comprises any one or more of ATP8B4, CD3D, CD5, CTSW, FAM102A, FASLG, GZMB, KLRC2, KLRC3, KLRC4, PRF1, SH2D1B, EBER, LMP1, and TNFRSF25. The PTCL subtype can be identified as ATLL if the gene expression profile comprises any one or more of ARSG, CCNE1, DOK5, FGF18, MYCN, NFATC1, NSMCE1, NUCB2, SAT1, SLC7A10, SPPL2A, STOM, TIAM2, UST, HBZ, and ZCCHC12.

The method of claim 2, wherein the PTCL subtype is identified as ALK+ALCL if the gene expression profile comprises any one or more of ALK, CACNA2D2, CCDC64, CCNA1, CCR4, DMBX1, GALNT2, GAS1, GATA3, HTRA3, ILIRAP, PCOLCE2, PFKFB3, RABGAP1L, TIAM2, TMEM158, and ZADH2. In certain embodiments, the PTCL subtype is identified as PTCL-NOS if the gene expression profile comprises any one or more of CASP2, EZH2, FLJ37453, IFI30, IFNG, LAGE3, LAP3, NR1H3, PLA2G7, SLAMF7, SLC31A2, TCN2, TMEM176B, WARS, TBX21, CXCR3, GATA3 and CCR4.

In embodiments, the sample is a biopsy specimen from the subject. The sample can comprises formalin-fixed paraffin-embedded tissue. In certain embodiments, the sample comprises fresh frozen tissue.

The method can further comprise providing the subject with an effective amount of a subtype-specific treatment. In embodiments, the sub-type specific treatment comprises a histone deacetylase (HDAC) inhibitor, an antifolate agent, an akylating agent, a protesome inhibitor, an antibody-drug conjugate, a phosphoinositide 3-kinases (PI3K) inhibitor, a Janus kinase (JAK) inhibitor, a signal transducer and activator of transcription (STAT) 3 inhibitor, a STAT5 inhibitor, an anaplastic lymphoma kinase (ALK) inhibitor, a hepatocyte growth factor (HGF) inhibitor, a cMET inhibitor, a platelet-derived growth factor receptor alpha (PDGFRα) inhibitor, a platelet-derived growth factor receptor beta (PDGFRβ) inhibitor, a mammalian target of rapamycin (mTOR) pathway inhibitor, an immune checkpoint inhibitor, a hypomethylating agent, an anti-cluster of differentiation 52 (CD52) antibody, an immunomodulatory drug, an anti-inducible T-cell costimulator (ICOS) antibody, a CC chemokine receptor 4 (CCR4) inhibitor, an isocitrate dehydrogenase (IDH) inhibitor, a B-cell lymphoma 2 inhibitor, an anti-interleukin-2 receptor alpha chain (CD25) antibody, a calcineurin inhibitor, a Notch signaling inhibitor, a Spleen tyrosine kinase (SYK) inhibitor, a bispecific antibody, a chimeric antigen receptor T (CAR-T) cell, or a combination thereof. In embodiments, the HDAC inhibitor comprises romidepsin, belinostat, panobinostat, or a combination thereof, the antifolate agents comprises pralatrexate; the akylating agent comprises bendamustine; the proteosome inhibitor comprises bortezomib; the antibody-drug conjugate comprises brentuximab vedotin; the PI3K inhibitor comprises duvelisib, tenalisib, or a combination thereof; the JAK inhibitor comprises ruxolitinib; the ALK inhibitor comprises crizotinib; the mTOR pathway inhibitor comprises everolimus; the hypomethylating agent comprises 5-azacytidine; the anti-CD52 antibody comprises alemtuzumab; the ImiD comprises lenalidomide; the CCR4 inhibitor comprises mogamulizumab; the IDH inhibitor comprises enasidenib; the BCL2 inhibitor comprises venetoclax; the anti-CD25 antibody comprises camidanlumab tesirine; the calcineurin inhibitor comprises cyclosporine A; the SYK inhibitor comprises cerdulatinib; the bispecific antibody comprises AFM13; the chimeric antigen receptor T (CAR-T) cell comprises CD30, CD7 or both; or a combination thereof.

The sub-type specific treatment for PTCL-NOS can comprise romidepsin, belinostat, brentuximab vedotin, duvelisib, or a combination thereof. The sub-type specific treatment for AITL can comprise romidepsin, 5-Aza, an isocitrate dehydrogenase (IDH) inhibitor, a calcineurin inhibitor, or a combination thereof. The sub-type specific treatment for ALK−ALCL can comprise brentuximab vedotin. The sub-type specific treatment for ALK+ALCL can comprise an ALK inhibitor, a platelet-derived growth factor receptor beta (PDGFRβ) inhibitor, or a combination thereof. The sub-type specific treatment for ATLL can comprise a NOTCH inhibitor, a hepatocyte growth factor (HGF) inhibitor, a cMET inhibitor, mogamulizumab, or a combination thereof. The sub-type specific treatment for ENKTL can comprise a platelet-derived growth factor receptor alpha (PDGFRα) inhibitor.

Another aspect of the present invention includes a diagnostic kit for identifying a subtype of Peripheral T-Cell Lymphoma (PTCL) in a sample from a subject, the kit comprising at least one of a means for detecting the presence of one or a combination of genes, representing a genetic signature that is indicative of a particular PTCL subtype and instructions for use. In embodiments, the PTCL subtype angioimmunoblastic T-cell lymphoma (AITL), anaplastic lymphoma kinase (ALK)-negative anaplastic large cell lymphoma (ALK−ALCL), adult T-cell leukemia/lymphoma (ATLL), extranodal natural killer/T-cell lymphoma (ENKTL), ALK-positive ALCL (ALK+ALCL), PTCL—not otherwise specified (PTCL-NOS), or indeterminate, wherein indeterminate indicates that the sample comprises features of at least two of the PTCL subtypes. The PTCL subtype can be indicative of AITL if the genetic signature comprises any one or more of ACHE, ADRA2A, ARRDC4, COL4A4, EFNB2, FCAMR, GJA4, GNA14, IER3IP1, ISG15, MSR1, OLFM1, PAPLN, S1PR3, SOX8, TMEM206, TUBB2B, and VANGL2. The PTCL subtype can be indicative of as ALK−ALCL if the genetic signature comprises any one or more of BATF3, C9orf89, CD28, DUSP2, ICMT, LGALS1, PIK3IP1, PRKCQ, TNFRSF8, TRAT1, and ZNF101. In embodiments, the PTCL subtype is indicative of ENKTL if the genetic signature comprises any one or more of ATP8B4, CD3D, CD5, CTSW, FAM102A, FASLG, GZMB, KLRC2, KLRC3, KLRC4, PRF1, SH2D1B, EBER, LMP1, and TNFRSF25. The PTCL subtype can be indicative of ATLL if the genetic signature comprises any one or more of ARSG, CCNE1, DOK5, FGF18, MYCN, NFATC1, NSMCE1, NUCB2, SAT1, SLC7A10, SPPL2A, STOM, TIAM2, UST, TAX, and ZCCHC12. The PTCL subtype can be indicative of ALK+ALCL if the gene signature comprises any one or more of ALK, CACNA2D2, CCDC64, CCNA1, CCR4, DMBX1, GALNT2, GAS1, GATA3, HTRA3, ILIRAP, PCOLCE2, PFKFB3, RABGAP1L, TIAM2, TMEM158, and ZADH2. The PTCL subtype can be indicative of PTCL-NOS if the gene signature comprises any one or more of CASP2, EZH2, FLJ37453, IFI30, IFNG, LAGE3, LAP3, NR1H3, PLA2G7, SLAMF7, SLC31A2, TCN2, TMEM176B, and WARS.

Another aspect includes a method of identifying a particular subtype of Peripheral T-Cell Lymphoma (PTCL) in a sample, the method comprising: obtaining a gene expression profile from a sample; comparing the gene expression profile to gene signatures associated with a particular PTCL subtype, wherein each subtype of PTCL comprises a unique gene signature; and identifying the subtype of PTCL within the sample as either angioimmunoblastic T-cell lymphoma (AITL), anaplastic lymphoma kinase (ALK)-negative anaplastic large cell lymphoma (ALK−ALCL), adult T-cell leukemia/lymphoma (ATLL), extranodal natural killer/T-cell lymphoma (ENKTL), ALK-positive ALCL (ALK+ALCL), PTCL—not otherwise specified (PTCL-NOS), or indeterminate, wherein indeterminate indicates that the gene expression profile of the sample comprises genes from the unique gene signature of at least two of the PTCL subtypes.

In embodiments, the gene signature of AITL comprises any one or more of ACHE, ADRA2A, ARRDC4, COL4A4, EFNB2, FCAMR, GJA4, GNA14, IER3IP1, ISG15, MSR1, OLFM1, PAPLN, S1PR3, SOX8, TMEM206, TUBB2B, and VANGL2; the gene signature of ALK−ALCL comprises any one or more of BATF3, C9orf89, CD28, DUSP2, ICMT, LGALS1, PIK3IP1, PRKCQ, TNFRSF8, TRAT1, and ZNF101; the gene signature of ENKTL comprises any one or more of ATP8B4, CD3D, CD5, CTSW, FAM102A, FASLG, GZMB, KLRC2, KLRC3, KLRC4, PRF1, SH2D1B, EBER, LMP1, and TNFRSF25; the gene signature of ATLL comprises any one or more of ARSG, CCNE1, DOK5, FGF18, MYCN, NFATC1, NSMCE1, NUCB2, SAT1, SLC7A10, SPPL2A, STOM, TIAM2, UST, TAX, and ZCCHC12; the gene signature of ALK+ALCL comprises any one or more of ALK, CACNA2D2, CCDC64, CCNA1, CCR4, DMBX1, GALNT2, GAS1, GATA3, HTRA3, ILIRAP, PCOLCE2, PFKFB3, RABGAP1L, TIAM2, TMEM158, and ZADH2; the gene signature of PTCL-NOS comprises any one or more of CASP2, EZH2, FLJ37453, IFI30, IFNG, LAGE3, LAP3, NR1H3, PLA2G7, SLAMF7, SLC31A2, TCN2, TMEM176B, and WARS; or a combination thereof.

In embodiments, identifying the subtype of PTCL comprises: a series of at least four binary predictors, wherein: a first binary predictor comprises determining whether the gene expression profile of the sample is more consistent with the genetic signature of AITL or PTCL-NOS; a second binary predictor comprises determining whether the gene expression profile of the sample is more consistent with the genetic signature of ALCL or PTCL-NOS; a third binary predictor comprises determining whether the gene expression profile of the sample is more consistent with the genetic signature of ATLL or PTCL-NOS; a fourth binary predictor comprises determining whether the gene expression profile of the sample is more consistent with the genetic signature of ENKTL or PTCL-NOS; and assigning the PTCL subtype of the specimen based on results from the binary predictors. The PTCL subtype can be identified as AITL when: in the first binary predictor, the gene expression profile of the sample is more consistent with the genetic signature of AITL; and in the second, third, and fourth binary predictors, the gene expression profile of the sample is more consistent with the genetic signature of PTCL-NOS. The PTCL subtype can be identified as ALCL when in the second binary predictor, the gene expression profile of the sample is more consistent with the genetic signature of ALCL; and in the first, third, and fourth binary predictors, the gene expression profile of the sample is more consistent with the genetic signature of PTCL-NOS. In one embodiment, the method further comprises a fifth binary predictor, wherein the fifth binary predictor comprises determining whether the gene expression profile of the sample is more consistent with the genetic signature of ALK+ALCL or ALK−ALCL; and identifying the PTCL subtype as ALK+ALCL when the gene expression profile of the sample is more consistent with the genetic signature of ALK+ALCL; or identifying the PTCL subtype as ALK−ALCL when the gene expression profile of the sample is more consistent with the genetic signature of ALK−ALCL.

In one embodiment, the PTCL subtype is identified as ATLL when: in the third binary predictor, the gene expression profile of the sample is more consistent with the genetic signature of ATLL; and in the first, second, and fourth binary predictors, the gene expression profile of the sample is more consistent with the genetic signature of PTCL-NOS. The PTCL subtype can be identified as ENKTL when: in the fourth binary predictor, the gene expression profile of the sample is more consistent with the genetic signature of ENKTL; and in the first, second, and third binary predictors, the gene expression profile of the sample is more consistent with the genetic signature of PTCL-NOS. The PTCL subtype can be identified as PTCL-NOS when in the first, second, third, and fourth binary predictors, the gene expression profile of the sample is more consistent with PTCL-NOS.

The PTCL subtype can be identified as indeterminate when at least two of the binary predictors are not more consistent with PTCL-NOS. In one embodiment, a sample identified as indeterminate undergoes an additional binary predictor, the additional binary predictor comprising: a first potential subtype that comprises one of the PTCL subtypes that was not more consistent with PTCL-NOS and a second potential subtype the comprises the at least one other PTCL subtype that was not more consistent with PTCL-NOS. In such embodiments, the method can further comprise: determining whether the gene expression profile of the sample is more consistent with the genetic signature of the first potential subtype or the second potential subtype; and identifying the PTCL subtype as the first potential subtype when the gene expression profile of the sample is more consistent with the genetic signature of the first potential subtype; or identifying the PTCL subtype the second potential subtype when the gene expression profile of the sample is more consistent with the genetic signature of the second potential subtype. If the PTCL subtype is identified as ALCL, the method can further comprises determining the ALCL subtype as either ALK+ALCL or ALK−ALCL using the fifth binary predictor as disclosed herein.

In embodiments, samples identified as PTCL-NOS cases are further segregated into GATA3- and TBX21-high subgroups. Samples identified as ENKTL can be subdivided into NK or gamma/delta T-cell lineages.

Another aspect includes a method of treating peripheral T-cell lymphoma. In embodiments, the method comprises: obtaining a gene expression profile from a sample of a subject; comparing the gene expression profile to gene signatures associated with a particular PTCL subtype, wherein each subtype of PTCL comprises a unique gene signature; identifying the subtype of PTCL within the sample as either angioimmunoblastic T-cell lymphoma (AITL), anaplastic lymphoma kinase (ALK)-negative anaplastic large cell lymphoma (ALK−ALCL), adult T-cell leukemia/lymphoma (ATLL), extranodal natural killer/T-cell lymphoma (ENKTL), ALK-positive ALCL (ALK+ALCL), PTCL—not otherwise specified (PTCL-NOS), or indeterminate, wherein indeterminate indicates that the gene expression profile of the sample comprises genes from the unique gene signature of at least two of the PTCL subtypes; and administering an effective amount of a therapeutic agent to the subject, wherein the therapeutic agent is configured to treat the identified PTCL subtype. In embodiments, the therapeutic agent comprises a histone deacetylase (HDAC) inhibitor, an antifolate agent, an akylating agent, a protesome inhibitor, an antibody-drug conjugate, a phosphoinositide 3-kinases (PI3K) inhibitor, a Janus kinase (JAK) inhibitor, a signal transducer and activator of transcription (STAT) 3 inhibitor, a STAT5 inhibitor, an anaplastic lymphoma kinase (ALK) inhibitor, a hepatocyte growth factor (HGF) inhibitor, a cMET inhibitor, a platelet-derived growth factor receptor alpha (PDGFRα) inhibitor, a platelet-derived growth factor receptor beta (PDGFRβ) inhibitor, a mammalian target of rapamycin (mTOR) pathway inhibitor, an immune checkpoint inhibitor, a hypomethylating agent, an anti-cluster of differentiation 52 (CD52) antibody, an immunomodulatory drug, an anti-inducible T-cell costimulator (ICOS) antibody, a CC chemokine receptor 4 (CCR4) inhibitor, an isocitrate dehydrogenase (IDH) inhibitor, a B-cell lymphoma 2 inhibitor, an anti-interleukin-2 receptor alpha chain (CD25) antibody, a calcineurin inhibitor, a Notch signaling inhibitor, a Spleen tyrosine kinase (SYK) inhibitor, a bispecific antibody, a chimeric antigen receptor T (CAR-T) cell, or a combination thereof.

In one embodiment, the HDAC inhibitor comprises romidepsin, belinostat, panobinostat, or a combination thereof; the antifolate agents comprises pralatrexate; the akylating agent comprises bendamustine; the proteosome inhibitor comprises bortezomib; the antibody-drug conjugate comprises brentuximab vedotin; the PI3K inhibitor comprises duvelisib, tenalisib, or a combination thereof; the JAK inhibitor comprises ruxolitinib; the ALK inhibitor comprises crizotinib; the mTOR pathway inhibitor comprises everolimus; the hypomethylating agent comprises 5-azacytidine (5-Aza); the anti-CD52 antibody comprises alemtuzumab; the ImiD comprises lenalidomide; the CCR4 inhibitor comprises mogamulizumab; the IDH inhibitor comprises enasidenib; the BCL2 inhibitor comprises venetoclax; the anti-CD25 antibody comprises camidanlumab tesirine; the calcineurin inhibitor comprises cyclosporine A; the SYK inhibitor comprises cerdulatinib; the bispecific antibody comprises AFM13; the chimeric antigen receptor T (CAR-T) cell comprises CD30, CD7 or both; or a combination thereof.

In embodiments, the therapeutic agent comprises romidepsin, belinostat, brentuximab vedotin, duvelisib, or a combination thereof when PTCL subtype is identified as PTCL-NOS. The therapeutic agent can comprises romidepsin, 5-Aza, an isocitrate dehydrogenase (IDH) inhibitor, a calcineurin inhibitor, or a combination thereof when the PTCL subtype is identified as AITL. The therapeutic agent can comprise NOTCH inhibitor, a hepatocyte growth factor (HGF) inhibitor, a cMET inhibitor, mogamulizumab when the PTCL subtype is identified as ATLL. In embodiments, the therapeutic agent comprises brentuximab vedotin when the PTCL subtype is identified as ALK−ALCL. The therapeutic agent can comprise an ALK inhibitor, a platelet-derived growth factor receptor beta (PDGFRβ) inhibitor, or a combination thereof when the PTCL subtype is identified as ALK+ALCL. In one embodiment, the therapeutic agent comprises a platelet-derived growth factor receptor alpha (PDGFRα) inhibitor when the PTCL subtype is identified as ENKTL.

### BRIEF DESCRIPTION OF THE DRAWINGS

The patent or application file contains at least one drawing executed in color. Copies of this patent or patent application publication with color drawings will be provided by the Office upon request and payment of necessary fee. Drawings described herein correspond to Amador C, Bouska A, Wright G, et at. Gene Expression Signatures for the Accurate Diagnosis of Peripheral f-Cell Lymphoma Entities in the Routine Clinical Practice. J Clin Oncol. 2022; 40(36):4261-4275. doi:10.1200/JCO.21.02707, which is incorporated by reference in its entirety.

Certain features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 shows molecular diagnostic signatures of PTCL subgroups. Unique gene expression signatures were identified for the major PTCL entities using a compound covariate prediction model (see ref nos. 10, 11 for details). Each column represents a PTCL patient and each row represents a unique gene of the classifier.

FIG. 2 shows two major molecular subgroups within PTCL-NOS. (A) A Bayesian predictor for the GATA3 and TBX21 subtypes was derived. Leave-one-out cross validation was used for classification precision. Approx. 18% of cases were not clearly defined (UC, unclassifiable). (B) Overall survival (OS) analysis of the GATA3 and TBX21 subtypes showed significant differences in clinical outcome (P=0.01) (Ref no. 10).

FIG. 3 shows error rate vs. number of genes in model. The Y-axis shows the average cross-validated error rate of 500 simulated data sets, with noise added to approximate the effect of a change of platform, as a function of the X axis, which indicates the number of candidate genes available. Each colored line represents a different diagnostic distinction (see legend in figure). There is a steep drop in the curves from left to right, with a flattening of each curve at 15-20 genes. These results demonstrate that this number of genes is sufficient to make each of the diagnostic distinctions and that additional genes do not further decrease the error rate.

FIG. 4 shows performance of the Lymph2Cx assay in an independent validation cohort. (A) The Lymph2Cx assay is shown in the form of a gene expression heat map with 67 patients. The 20 genes that contribute to the model are shown at the left, including 5 housekeeping genes. The cell-of-origin assignments are shown for the assay and compared with the gold standard method, which uses previously published algorithms on gene expression from fresh frozen tissue and 3 immunohistochemistry-based algorithms^{20,23}. (B) Comparison of the Lymph2Cx scores in the validation cohort from two independent laboratories (MoCha, CLC). The horizontal and vertical light gray lines represent the thresholds between the GCB, unclassified, and ABC subtypes. The R2 is 0.996 and the slope of the line of best-fit is 1.015. MoCha, Molecular Characterization Laboratory, Frederick National Laboratory for Cancer Research (Frederick, MD); CLC, Centre for Lymphoid Cancer, BC Cancer Agency (Vancouver, BC).

FIG. 5 includes (A) Histogram showing distribution of correlations between Nanostring vs. Affymetrix data for 667 diagnostic and prognostic genes in DLBCL. (B) Genes present in “academic” PTCL signature. The majority of the 40 genes are well correlated, with a median correlation of 0.76.

FIG. 6 shows assay development using U133 array data from FF samples independent of the validation set to develop a linear predictor of subtypes including cut-points. In the training set, replicate samples will be used to adjust gene weights and additive shift to account for platform differences and to mimic the FF predictor score as closely as possible with FFPE specimens/NanoString analysis, establishing a locked version for validation. Phase II studies that will follow the current proposal are also indicated.

FIG. 7 shows variability (Y-axis) versus Signal Intensity (X-axis). Each colored line represents data from a different Lymphoma/Leukemia Molecular Profiling Project (LLMPP) institution. Overlap of the lines indicates excellent reproducibility between institutions, with some variability at the lowest signal intensities.

FIG. 8 shows exemplary immunohistochemical staining for sub-classification of ALCL and PTCL-NOS. ALCL can be sub-classified as CD30+ or ALK+, and GATA3 and TBX21 represent two subgroups of PTCL-NOS.

FIG. 9 provides a histogram comparison of RNA yield from FFPE tissues collected at varying times using different protocols for DNA/RNA isolation. RNA isolation was performed using either the STORM protocol, a modified STORM protocol, or Quiagen protocol. Recovery of RNA from older blocks was generally lower, but was not different using newer kits.

FIG. 10A includes representative gel images of RNA isolated from FFPE and Fresh Frozen samples using varying protocols. The tissues were collected in the year indicated.

FIG. 10B provides a histogram comparing the effectiveness of different protocols in isolating larger RNA molecules (greater than 200 base pairs in length) from samples of varying ages.

FIG. 11 shows a refined gene signature with additional genes added in the diagnostic signature list as compared to FIG. 1 (modified from Ref 10).

FIG. 12 provides PTCL Molecular Classifier. FIG. 12A provides the study design and schematics of the molecular diagnosis of PTCL. A molecular classifier for PTCL subclassification was derived using HG-U133plus2.0 array data from PTCL with fresh frozen (FF) tissue (n=109), designated as the training cohort. This molecular classifier had 442 distinct genes, including housekeeping and other genes involved in T-cell biology. This transcriptomic signature was considered for nCounter® analysis (NanoString, Inc) using corresponding matched FFPE samples. Transcripts that showed a high correlation between FF and FFPE data were selected. The algorithm was further refined to have the minimum number of transcripts for subclassification and mimic the FF predictor score with the NanoString platform (see M&M for details). The final diagnostic model resulted in 153 transcripts (99 diagnostic, 5 viral and 16 housekeeping and 33 T-cell biology related) and was validated in an independent cohort of PTCL cases rigorously characterized by pathology and other ancillary methods. The classification algorithm was based on a series of several binary predictors to distinguish one entity from another, as detailed in FIG. 17. FIG. 12B provides a heatmap of the finalized nCounter® classifier in the training and the validation cohorts. EBV and HTLV-1 viral transcripts commonly expressed in specific PTCL subtypes are shown, and housekeeping genes used for normalization and that do not vary between PTCL subtypes are displayed for comparison. FIG. 12C shows a Kaplan-Meier curve of overall survival for 89 of the 105 training cohort cases and 66 of the 140 validation cases with available outcome data. FIG. 12D shows Kaplan-Meier curve of OS of PTCL subtypes included in training cohort (molecular classification by HG-U133plus2.0 array). FIG. 12E shows Kaplan-Meier curve of OS of PTCL entities included in the validation cohort (pathology classification).

FIG. 13 shows AITL Classification. FIG. 13A provides a scatterplot of the AITL diagnostic score vs. the average expression of 5 TFH related genes in training and validation AITLs. FIG. 13B provides a boxplot of EBER and EBNA1 transcript expression in the AITLs (training/validation cohort). FIG. 13C provides a Kaplan-Meier curve of OS of AITLs in the combined cohorts by CD20 mRNA expression. Solid lines are cases classified as AITL by molecular classification (p=0.02); dotted lines are AITL by pathology (p=0.06). CD20 expression in a representative low and high expression AITL case (400×). FIG. 13D shows the mutation status of cases with available sequencing data. Cases that were AITL-PTCL-NOS intermediate or did not classify as AITL on the NanoString classifier are noted with an asterisk. FIG. 13E provides a violin and dot plot of AITL classification diagnostic scores in AITL and PTCL-NOS cases profiled on the nCounter®. Cases that were discordant between AITL and PTCL-NOS are colored in red and intermediate AITL cases in grey. FIG. 13F provides heatmaps of AITL showing disagreement by nCounter® classification in the validation cohort. The mean signature of the concordant cases is shown. For cases labeled intermediate, those diagnosed as AITL by consensus pathology review are on the left, and intermediate cases diagnosed as PTCL-NOS are on the right. 2 PTCL-NOS classified as AITL by nCounter®. Figures G-I provide an example of focal expression of BCL6 and ICOS (400×; G, H&I; B, BCL-6 and C, ICOS) seen in a PTCL-NOS case that classified as AITL by nCounter® platform.

FIG. 14 shows ALCL Classification. FIG. 14A provides a violin and scatter plot of ALCL classification scores vs. PTCL-NOS. FIG. 14B shows ALK-positive ALCL versus ALK-negative ALCL on the nCounter® platform. Cases that were discordant between ALCL and PTCL-NOS or ALK-negative and ALK-positive are colored in red. FIG. 14C provides heatmaps of ALK-negative and ALK-positive ALCL cases showing disagreement by nCounter® classification in the validation cohort. H and E and CD30 and ALK immunostains of a representative ALK-negative ALCL case that classified as ALK-positive ALCL by NanoString Assay are shown (400×). FIG. 14D provides a Kaplan-Meier curve of OS of ALCLs in the training and validation cohort by NanoString Classification. FIG. 14E provides a heatmap of CD30 and cytotoxic transcript expression in ALCL and PTCL-NOS cases. Discrepant cases are noted with red lines.

FIG. 15 shows ATLL and ENKTCL Classification. FIG. 15A provides a violin and dot plot of ATLL classification scores in ATLL and PTCL-NOS cases profiled on the nCounter®. FIG. 15B provides heatmaps of ATLL cases discordant by nCounter® classification in the validation cohort. H&E and CD4 staining for a case diagnosed as PTCL-NOS but classified at ATLL by nCounter® (lower panel). FIG. 15C provides a scatterplot of expression of the HTLV-1 specific transcripts HBZ vs. ATLL score in training and validation ATLL cases. Solid fitted line represents training data and dashed line validation data. FIG. 15D provides a heatmap of HBZ and TAX expression in the ATLL and PTCL-NOS validation cohorts. The discrepant cases are noted by a red asterisk. FIG. 15E provides a scatterplot of HBZ expression measured by qPCR vs. nCounter®. FIGS. 15F-G provide violin and dotplots of ENKTCL classification scores (F) or EBER scores (G) in ENKTCLs and PTCL-NOS cases profiled in the training and validation cohorts on the nCounter®. Discordant cases are red. FIG. 15H provides a heatmap of expression of CD3 gamma and delta and EBV transcripts in ENKTCL and PTCL-NOS cases in the training and validation cohorts. FIG. 15I provides a heatmap of expression of relevant signatures in the ENKTCL discordant case compared to average signatures in the validation cohort.

FIG. 16 shows PTCL-NOS Subclassification. FIG. 16A provides violin and dot plots of PTCL-GATA3 classification scores in PTCL-NOS cases profiled in the training and validation cohorts on the nCounter®. FIG. 16B shows the mutation status of PTCL-NOS cohort with available sequencing data. FIG. 16C provides heatmaps of expression of CD4, CD8, CD20, and cytotoxic genes in the training (upper) and validation (lower) cases. FIG. 16D provides a scatterplot of the average expression of the cytotoxic genes vs. CD20 in cases that classified as PTCL-TBX21 by NanoString. FIG. 16E provides H&E and IHC stains for one representative PTCL-GATA3 case showing GATA3 (left) and one PTCL-TBX21 case showing TBX21 and CD8 expression (right). FIG. 16F shows a KM curve of overall survival for PTCL-NOS cases with available outcome data in the combined training and validation cohorts classified as PTCL-GATA3 or PTCL-TBX21 NanoString.

FIG. 17 provides a schematic of algorithm design for PTCL-subclassification: Six pairwise sub-models are combined to generate a final predictor for classification. Stage-1: Initially four categories (AITL, ALCL, ENKTCL, or ATLL) are distinguished from PTCL-NOS. Subsequently EBV-viral transcript (EBER) is added to the ENKTCL classifier (versus PTCL-NOS) and finally ALCL is subclassified into ALK−ALCL vs ALK+ALCL. Stage-2: In the second stage samples considered as PTCL-NOS are distinguished into PTCL-GATA3 or PTCL-TBX21 subtypes. If more than one of the 4 predictors resulted in a non-PTCL-NOS diagnosis (e.g., ALCL and AITL), then an additional head-to-head predictor between those types is applied to break the tie (e.g., ALCL vs. AITL), with all genes characteristic of either type selected to be included in the model.

FIG. 18A provides a scatter plot of log 2(counts) measured by the nCounter442 gene Assay (NanoString, Inc) vs log 2 (probe-Intensity) value for the corresponding transcript measure by HG-U133 plus2 (Affymetrix, Inc) for the same RNA extracted from fresh frozen tissue. FIG. 18B provides a histogram of the correlation values for each mRNA transcript for 10 cases profiled by Two planforms (nCounter, NanoString, Inc vs and HG-U133 plus 2 (Affymetrix, Inc). FIG. 18C provides a gel image comparing RNA extracted by the Qiagen (Q) or the Storm Kit (S). FIG. 18D provides a bar graph comparing the % of RNA>200 bases in RNA extracted by the Qiagen (Q) or the Storm Kit (S). FIG. 18E provides scatter plots and histograms comparing log 2(counts) generated by nCounter analysis of RNA from fresh frozen (FF) or FFPE extracted using Qiagen or Storm kits from a corresponding biopsy. The number in the boxes represent the correlation coefficient. FIG. 18F provides a scatter plot of log 2(counts) generated by nCounter analysis of RNA extracted from FF or FFPE tissue from a corresponding biopsy.

FIG. 19A provides a histogram of the correlation values for each mRNA transcript for FFPE RNA analyzed by nCounter(NanoString, Inc) and FF RNA analyzed by HG-U133 plus 2 (Affymetrix, Inc) for 100 cases profiled both platform. FIG. 19B provides a histogram of the correlation values for each mRNA transcript in the classifiers for FFPE RNA analyzed by nCounter and FF RNA analyzed by HG-U133 plus 2 for 100 cases profiled both platforms. FIG. 19C-D provides heatmaps of Affymetrix U133 plus2 data from FF RNA from cases included in the training cohort depicting the original, but extended classifier gene lists (C) and reduced classifier gene lists (D) ultimately used in the nCounter platform classification model. Housekeeping genes that do not vary between PTCL subtypes are displayed for comparison. FIG. 19E provides a boxplot and dot plot of variances of housekeeping and model genes analyzed in FFPE tissue by nCounter in training and validation cases run. FIG. 19F provides a Kaplan-Meier curve of overall survival of PTCL-NOS cases classified as GATA3 or TBX21 using FF RNA analyzed by HG-U133 plus2 Affymetrix array (upper) or FFPE RNA analyzed by nCounter (lower).

FIG. 20 shows a comparison of model scores (FFPE RNA) generated at UNMC (initial design) vs model scores on finalized diagnostic model run at 3 different sites (UNMC, Insight. Inc, and COH). The threshold for classifying a case as an PTCL entity are labeled in green. The light green line depicts the threshold for Intermediate AITL/PTCL-NOS score. Red line indicates the results of an orthogonal regression, which can be compared to the grey line indicating the diagonal of equal model scores. *nCounterpanel using the reduced gene design containing 153 CodeSets. **nCounterpanel using the initial design containing 442 CodeSets. All cases were run on the original 442 CodeSetdesign at UNMC and run on the finalized 153 CodeSetdesign at other sites, while 7 samples were also repeated at UNMC using the 153 finalized design CodeSetpanel. All scores were generated using the finalized model.

FIG. 21 provides violin and dotplots of classification scores in all PTCL cases profiled in the Training and Validation cohorts on the nCounter. The x axis is the known diagnosis. Cases where the nCounter classification did not agree with the expected diagnosis are colored according to how they classified, while concordant cases are grey.

FIG. 22 shows H&E and IHC stains for one of the nodal ENKTL cases with strong EBER and cytotoxic (TIA-1 and perforin) marker expression

FIG. 23A provides a heatmap of TFH, AITL, GATA3 and TBX21 signature expression in AITL, PTCL-TFH, and PTCL-TBX21, PTCL-GATA3, and reactive hyperplasia. FIG. 23B provides a boxplot of the average expression of TFH specific genes in the noted entities.

FIG. 24 shows concordance between gold standard diagnosis and refined diagnostic signature in training cohort. AITL, angioimmunoblastic T-cell lymphoma; ALCL, anaplastic large cell lymphoma; ATLL, adult T-cell lymphoma/leukemia; ENKTCL, extranodal NK/T-cell lymphoma; PTCL-NOS, peripheral T-cell lymphoma, not otherwise specified; FFPE, formalin fized paraffin embedded tissue; FF, fresh frozen tissues. *Borderline cases were not considered discordant ^{#}Combination of pathological and molecular diagnosis based on fresh frozen GEP.

FIG. 25 shows concordance between gold standard diagnosis and refined diagnostic signature in validation cohort. The transcriptional classifier matched the pathology diagnosis rendered by 3 expert hematopathologists in 85% (119/140) of the cases and showed a “borderline association” with the molecular signatures in 6% (8/140). Overall, 127/140 cases (in green) are considered concordant between consensus pathology review and molecular diagnosis. The classifier improved the pathology diagnosis in 2 cases PTCL-NOS cases (aqua) that were molecularly classified as ATLL which at re-examination were concordant with subsequent clinicopathological information. Among the remaining 11 cases with “disagreements”, 4 had a molecular classification (yellow) that may provide an improvement over pathologic diagnosis based on the overall transcriptomic signature and morphological features. AITL, angioimmunoblastic T-cell lymphoma; ALCL, anaplastic large cell lymphoma; ATLL, adult T-cell lymphoma/leukemia; ENKTCL, extranodal NK/T-cell lymphoma; PTCL-NOS, peripheral T-cell lymphoma, not otherwise specified *Borderline cases were not considered discordant ⁺PTCL-NOS cases molecularly classified as ATLL which at re-examination were concordant with subsequent clinicopathological information ⁺⁺PTCL-NOS cases molecularly classified as AITL which at re-examination showed TFH marker expression ⁺⁺⁺ALK−ALCL cases that based on the overall transcriptomic signatures may represent ALK-positive-like ALCL.

### DESCRIPTION OF EXEMPLARY EMBODIMENTS

The details of one or more embodiments of the presently disclosed subject matter are provided herein. It is to be understood, however, that the presently disclosed subject matter can be embodied in various forms. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and not to be interpreted as limiting. In case of conflict, the specification of this document, including definitions, will control.

While the terms used herein are believed to be well understood by those of ordinary skill in the art, certain definitions are set forth to facilitate explanation of the presently disclosed subject matter. Unless defined otherwise, all technical and scientific terms used herein have the same meaning as commonly understood by one of ordinary skill in the art to which the presently disclosed subject matter belongs.

Although any methods, devices, and materials similar or equivalent to those described herein can be used in the practice or testing of the presently disclosed subject matter, representative methods, devices, and materials are now described.

In certain instances, nucleotides and polypeptides disclosed herein are included in publicly-available databases, such as GENBANK® and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

Unless defined otherwise, all technical and scientific terms used herein have the same meaning as is commonly understood by one of skill in the art to which the invention(s) belong. All patents, patent applications, published applications and publications, GenBank sequences, databases, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently disclosed subject matter, representative methods, devices, and materials are now described.

Following long-standing patent law convention, the terms “a” “an”, and “the” refer to “one or more” when used in this application, including the claims. Thus, for example, reference to “a cell” includes a plurality of such cells, and so forth.

The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, a “biological sample” refers to a sample of biological material obtained from a subject. In embodiments, the subject comprises a human subject. Examples of a biological sample include a tissue, a tissue sample, a cell sample, a fluid sample, or a combination thereof. A biological sample can be obtained in the form of e.g., a tissue biopsy, such as, an aspiration biopsy, a brush biopsy, a surface biopsy, a needle biopsy, a punch biopsy, an excision biopsy, an open biopsy, an incision biopsy and an endoscopic biopsy. “Biological sample” can refer to a sample of tissue or fluid isolated from a subject, including but not limited to, for example, blood, plasma, serum, tumor biopsy, urine, stool, sputum, spinal fluid, pleural fluid, nipple aspirates, lymph fluid, the external sections of the skin, respiratory, intestinal, and genitourinary tracts, tears, saliva, milk, cells, tumors, organs, and also samples of in vitro cell culture constituent. Non-limiting example of the biological sample includes formalin-fixed paraffin-embedded (FFPE) tissue, fresh frozen (FF) samples, or other prepared sample types.

The skilled artisan will recognize that a biological sample from a subject can be obtained by a variety of convention techniques. The methods described herein typically involve obtaining a biological sample from the subject, such as a subject suspected of having cancer or a subject afflicted with cancer. As used herein, the phrase “obtaining a biological sample” refers to any process for directly or indirectly acquiring a biological sample from a subject. For example, a biological sample can be obtained (e.g., at a point-of-care facility, e.g., a physician's office, a hospital, laboratory facility) by procuring a tissue or fluid sample (e.g., tissue biopsy, blood draw, marrow sample, spinal tap) from a subject. Alternatively, a biological sample can be obtained by receiving the biological sample (e.g., at a laboratory facility) from one or more persons who procured the sample directly from the subject. The biological sample can be, for example, a tissue (e.g., tissue biopsy, blood), cell (e.g., hematopoietic cell such as hematopoietic stem cell, leukocyte, or reticulocyte, stem cell, or plasma cell, cancer cell), vesicle, biomolecular aggregate or platelet from the subject.

In an embodiment, the sample is from a resection, biopsy, or core needle biopsy of a primary or metastatic tumor. In addition, fine needle aspirate samples are used. Samples can be either paraffin-embedded or frozen tissue.

In embodiments, the biological sample can be a biopsy tissue, wherein the biopsy tissue contains at least one cell that is a cancer cell or suspected of being a cancer cell. The term “biopsy tissue” can refer to a sample of tissue that is removed from a subject for the purpose of determining if the sample contains cancerous tissue, or examining a tissue that is cancerous. In some embodiment, biopsy tissue is obtained because a subject is suspected of having cancer. The biopsy tissue is then examined for the presence or absence of cancer. In other embodiments, biopsy tissue is obtained because a subject is known to have cancer, and then the biopsy tissue is then examined for markers that indicate cancer stage and/or treatment options.

As used herein, “diagnose” refers to detecting and identifying a disease in a subject. The term can also encompass assessing or evaluating the disease status (progression, regression, stabilization, response to treatment, etc.) in a patient known to have the disease (e.g. PTCL).

As used herein, the term “prognosis” refers to providing information regarding the impact of the presence of cancer (e.g., as determined by the diagnostic methods of the present invention) on a subject's future health (e.g., expected morbidity or mortality, the likelihood of getting cancer, and the risk of metastasis). In other words, the term “prognosis” refers to providing a prediction of the probable course and outcome of a cancer or the likelihood of recovery from the cancer. The term “prognosis” is recognized in the art and encompasses predictions about the likely course of disease or disease progression, particularly with respect to likelihood of disease remission, disease relapse, tumor recurrence, metastasis, and death. A “good prognosis” can refer to the likelihood that a patient afflicted with cancer will remain cancer-free after therapy. A “poor prognosis” can refer to the likelihood of a relapse or recurrence of the underlying cancer after treatment, the likelihood of developing metastases, and/or the likelihood of death. In particular embodiments, the time frame for assessing prognosis is, for example, less than one year, one, two, three, four, five, six, seven, eight, nine, ten, fifteen, twenty, or more years.

The term “treat” “treatment” or “treating” as used herein refers to any type of treatment that imparts a benefit to a patient afflicted with a disease, including improvement in the condition of the patient (e.g., in one or more symptoms), delay in the progression of the condition, etc.

Aspects of the invention refer to a method for the treatment of cancer in a patient in need thereof. Treatment of cancer, as used herein, can refer to partially or totally inhibiting, delaying or preventing the progression of cancer, including cancer metastasis; inhibiting, delaying or preventing the recurrence of cancer; or preventing the onset or development of cancer (chemoprevention) in a mammal, for example a human. Treating cancer can be indicated by stopping or reducing tumor development, tumor growth, proliferation, angiogenesis, and/or metastasis.

Aspects of the invention also comprise providing diagnostic information for a cancer in a subject. For example, aspects of the invention can comprise providing diagnostic information to a subject suspected of having cancer, or a subject at risk of having cancer. The term “subject suspected of having cancer” can refer to a subject that presents one or more symptoms indicative of a cancer (e.g., a noticeable lump or mass) or is being screened for a cancer (e.g., during a routine physical). A subject suspected of having cancer can also have one or more risk factors. A subject suspected of having cancer has generally not been tested for cancer. However, a “subject suspected of having cancer” encompasses an individual who has received an initial diagnosis but for whom the stage of cancer is not known. The term further includes people who once had cancer (e.g., an individual in remission). As used herein, the term “subject at risk for cancer” refers to a subject with one or more risk factors for developing a specific cancer. Risk factors include, but are not limited to, gender, age, genetic predisposition, environmental expose, previous incidents of cancer, preexisting non-cancer diseases, and lifestyle.

The phrase “effective amount” refers to that amount of therapeutic agent that results in an improvement in the patient's condition. In certain embodiments, the effective amount increases survival rate by at least one year. The effective amount can be measured by the years of survival after initiating treatment in which the disease does not substantially progress. In embodiments, the effective amount can be an amount sufficient to cause at least one year of progression-free survival (PFS). The effective amount can be sufficient to induce more than one year of PFS. In embodiments, the effective amount is a dosage sufficient to cause a 1-year PFS, 2-year PFS, 3-year PFS, 4-year PFS, or 5-year PFS. In embodiments, the therapeutic agent is an anti-cancer agent.

As used herein, the term “subject” refers to a target of administration. The subject of the herein disclosed methods can be a vertebrate, such as a mammal, a fish, a bird, a reptile, or an amphibian. Thus, the subject of the herein disclosed methods can be a human or non-human. Thus, veterinary therapeutic uses are provided in accordance with the presently disclosed subject matter.

“Subject” can refer to mammals such as humans and non-human primates, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; rabbits, guinea pigs, and rodents. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

In embodiments, the subject has been previously diagnosed as carrying a cancer, and possibly has already undergone treatment for the cancer. In alternative embodiments, the subject has not been previously diagnosis as carrying a cancer. The present invention can be useful with all patients at risk for a cancer. Although each type of cancer has their own set of risk factors, the risk of developing cancer increases as with aged, gender, race and personal and family medical history. Other risk factors are largely related to lifestyle choices, while certain infections, occupational exposures and some environmental factors can also be related to developing cancer.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraoral administration, intracerebral administration, rectal administration, and parenteral administration, including injectable such as intravenous administration, intra-arterial administration, intramuscular administration, and subcutaneous administration. Administration can be continuous or intermittent. A preparation can be administered therapeutically; that is, administered to treat an existing condition of interest. A preparation can be administered prophylactically; that is, administered for prevention of a condition of interest.

The presently disclosed subject matter relates to methods for diagnosis, prognosis, or treatment of cancer in a subject by determining the presence or amount of one or more biomarkers in a biological sample from a subject. In embodiments, the cancer is non-Hodgkin lymphoma. The cancer can be PTCL.

In an embodiment, the method comprises obtaining a biological sample from a subject, wherein the biological sample comprises at least one cell that is a cancer cell or suspected of being a cancer cell; measuring or determining the expression level of genes from the biological sample; comparing the expression level in the biological sample to a control sample; and providing diagnostic, prognostic, or predictive information based on the measuring step.

In embodiments, a measurement, such as a level of nucleotides or protein of a gene, can be compared to a control and, if varying from that of the control, the subject can have an increased likelihood of having and/or developing cancer, a decreased likelihood of having or developing cancer, an increased likelihood of responding to a given treatment, or a decreased likelihood of responding to a given treatment. “varying from that of a control” sample or subject is understood as having a level of the analyte or diagnostic or therapeutic indicator (e.g., marker) to be detected at a level that is statistically different than a sample from a normal, untreated, or abnormal state control sample. Determination of statistical significance is within the ability of those skilled in the art, e.g., the number of standard deviations from the mean that constitute a positive or negative result. For example, the control sample can comprise one or more non-cancerous cells, or a biological sample form a patient who has not been diagnosed with a cancer.

In embodiments, a measurement, such as the level of mRNA and/or protein can be compared to a threshold to determine if a subject has cancer, to diagnose the type or subtype of a cancer, to provide a prognosis for the patient, to determine a specific treatment regimen, or a combination thereof. The term “threshold” can refer to a value derived from a plurality of biological samples for a biomarker above which threshold is associated with an increased likelihood of having and/or developing cancer, or an increased likelihood of responding to a given treatment.

According to certain embodiments of the inventive methods, the presence or amount of a gene product, e.g., a polypeptide or a nucleic acid, encoded by a gene is detected in a sample derived from a subject (e.g., a sample of tissue or cells obtained from a tumor or a blood sample obtained from a subject). The sample can be subjected to various processing steps prior to or in the course of detection.

For the purposes of the present invention, the term “gene” has its meaning as understood in the art. However, it will be appreciated by those of ordinary skill in the art that the term “gene” has a variety of meanings in the art, some of which include gene regulatory sequences (e.g., promoters, enhancers, etc.) and/or intron sequences, and others of which are limited to coding sequences. It will further be appreciated that definitions of “gene” include references to nucleic acids that do not encode proteins but rather encode functional RNA molecules such as tRNAs. For the purpose of clarity we note that, as used in the present application, the term “gene” generally refers to a portion of a nucleic acid that encodes a protein; the term can optionally encompass regulatory sequences. This definition is not intended to exclude application of the term “gene” to non-protein coding expression units but rather to clarify that, in most cases, the term as used in this document refers to a protein coding nucleic acid.

A gene product or expression product is, in general, an RNA transcribed from the gene or a polypeptide encoded by an RNA transcribed from the gene.

Expression of a gene can be measured by a variety of techniques known in the art. Certain techniques can make use of a polynucleotides corresponding to part or all of the gene rather than an antibody that binds to a polypeptide encoded by the gene. Appropriate techniques include, but are not limited to, in situ hybridization, Northern blot, and various nucleic acid amplification techniques such as PCR, quantitative PCR, and the ligase chain reaction.

In some embodiments, the present application provides a method of identifying distinct subgroups PTCL based on the presence or amount of one or more biomarkers in a biological sample. Non-limiting subgroups of PTCL include PTCL-NOS, AITL, ALCL, ALK(−) ALCL, ENKTL, NK and γδ-T-PTCL. In embodiments, PTCL-NOS can be further subdivided into one of two major molecular subtypes characterized by either high expression of the transcription factors GATA3 or TBX21 and their target genes.

In certain embodiments, the biomarkers comprise genetic signatures that are uniquely present in different subtypes of PTCL. The genetic signatures for each subtype of PTCL can include one or more of the genes listed in Table 1, below.

TABLE 1
Exemplary PTCL Subtype Gene Signatures

| | ALK- | ALK- | | | | |
|---|---|---|---|---|---|---|
| | NEGATIVE | POSITIVE | | | | |
| AITL | ALCL | ALCL | ENKTL | ATLL | GATA3/TBX21 | HOUSEKEEPING |
| ACHE | BATF3 | ALK | ATP8B4 | ARSG | CASP2 | CLPX |
| ADRA2A | C9orf89 | CACNA2D2 | CD3D | CCNE1 | EZH2 | COX15 |
| ARRDC4 | CD28 | CCDC64 | CD5 | DOK5 | FLJ37453 | FAM120A |
| COL4A4 | DUSP2 | CCNA1 | CTSW | FGF18 | IFI30 | FBXO7 |
| EFNB2 | ICMT | CCR4 | FAM102A | MYCN | IFNG | FBXO9 |
| FCAMR | LGALS1 | DMBX1 | FASLG | NFATC1 | LAGE3 | GUSB |
| GJA4 | PIK3IP1 | GALNT2 | GZMB | NSMCE1 | LAP3 | MARK3 |
| GNA14 | PRKCQ | GAS1 | KLRC2 | NUCB2 | NR1H3 | MAX |
| IER3IP1 | TNFRSF8 | GATA3 | KLRC3 | SAT1 | PLA2G7 | OGFOD1 |
| ISG15 | TRAT1 | HTRA3 | KLRC4 | SLC7A10 | SLAMF7 | PAFAH1B2 |
| MSR1 | ZNF101 | IL1RAP | PRF1 | SPPL2A | SLC31A2 | PRPSAP1 |
| OLFM1 | | PCOLCE2 | SH2D1B | STOM | TCN2 | RAB21 |
| PAPLN | | PFKFB3 | TNFRSF25 | TIAM2 | TMEM176B | RAF1 |
| S1PR3 | | RABGAPIL | EBNA1 | UST | WARS | TIAL1 |
| SOX8 | | TIAM2 | CD3G | ZCCHC12 | GATA3 | TOX4 |
| TMEM206 | | TMEM158 | EBER | HBZ | TBX21 | YY1 |
| TUBB2B | | ZADH2 | | TAX | CXCR3 | |
| VANGL2 | | | | | CCR4 | |
| CXCL13 | | | | | EOMES | |
| PDCD1 | | | | | | |

In some embodiments, the one or more biomarkers include GATA, TBX21, or their associated target genes. The one or more biomarkers can include NK or gamma/delta T-cell lineages. In certain embodiments, NK lineage comprise EBV viral transcription (EBER and LMP1). In embodiments, the gene signature for the ATLL subtype further comprises HTLV-1 viral transcript expression (TAX, HBZ, or a combination thereof).

In some embodiments, the present application provides a method of diagnosis, prognosis, and/or treatment of cancer in a subject by determining expression profiles of a specific panel of genes.

In some embodiments, the present application provides a method of predicting the survival of a subject diagnosed with specific subgroup of PTCL. In some embodiments, the method relates to detecting the expression of profiles of specific panels of genes.

Further provided, in some embodiments of the presently disclosed subject matter, is a method for treatment for a specific subgroup of PTCL in a subject. In embodiments, the method of treatment comprises and administering to the subject an effective amount of an anti-cancer agent that to the subject. The anti-cancer agent can vary depending on the subject's PTCL subtype. In embodiments, the treatment is optimized to specifically target the subject's PTCL subtype. Non-limiting examples of such agents comprise chemotherapy, immunotherapy, toxin therapy, radiotherapy, or a combination thereof. The anti-cancer agents can comprise pralatrexate, romidepsin, belinostat, brentuximab vedotin, duvelisib, decitabine and related compounds, EZH1/2 inhibitors, other PTCL-subtype specific treatments currently known or later discovered, or combinations thereof,

Additionally, the presently disclosed subject matter relates to a kit and reagents for diagnosis, prognosis, and/or treatment of PTCL. In some embodiments, the kit includes a means of detecting expression profiles of specific panels of genes. In some embodiments, the kit comprises one or more probes.

Further, the presently disclosed subject matter relates to methods of identification of a set biomarkers for diagnosis, prognosis and/or treatment of PTCL using software and analytical systems. In some embodiments, the presently disclosed subject matter provides a method of subtyping PTCL by comparing expression levels of a number of genes.

Embodiments employ methodology to analyze a large number of nucleic acids in a single reaction. In embodiments, the methods or kits employ high-throughput nucleic acid sequencing technologies such as next generation sequencing (NGS). In embodiments, a targeted RNA-sequence panel can be offered on an NGS platform. Non-limiting examples of such NGS technologies include instruments and protocols from Illumina, Inc (San Diego, CA, USA), Thermo Fischer Scientific (Waltham, MA, USA), Qiagen (Venlo, Netherlands). In still other embodiments, certain multiplex PCR-based platforms are employed. Embodiments use qPCR-based platforms configured to accommodate a substantial number of analytes, such as the Qiagen Modaplex (Venlo, Netherlands) or similar platform. Embodiments can employ conventional real time PCR platforms. Alternative embodiments utilize any of various expression microarray-based platforms, including, but not limited to those from Affymetrix (Santa Clara, CA, USA), Aknonni Biosystems (Frederick, MD, USA), Biofire Diagnostics (Salt Lake City, UT, USA) or similar platforms. Some embodiments employ non-array platforms and protocols. In certain non-array embodiments, a quantitative nuclease protection assay is utilized, Other non-array embodiments employ platforms and protocols from NanoString Technologies, Inc. (Seattle, WA, USA). The examples provided herein are merely exemplary and should not be considered limiting in any way. Embodiments employ any of various molecular diagnostic platforms.

Certain embodiments employ a DNA chip, which is a device that is convenient to compare expression levels of a number of genes at the same time. DNA chip-based expression profiling can be carried out, for example, by the method as disclosed in “Microarray Biochip Technology” (Mark Schena, Eaton Publishing, 2000), etc.

A DNA chip comprises immobilized high-density probes to detect a number of genes. Thus, expression levels of many genes can be estimated at the same time by a single-round analysis. Namely, the expression profile of a specimen can be determined with a DNA chip.

In some embodiments, the presently disclosed subject matter relates to a method of identifying biomarker that are specific for different subtypes of cancer, such as PTCL. Embodiments comprise the steps of performing a diagnostic algorithm based on GEP analysis of biological samples. Certain embodiments employ a step-wise binary model algorithm for subgroup classification. Embodiments provide a method of substantially reducing the number of genes in a cancer subgroup classifier without significantly compromising diagnostic accuracy. In embodiments, the total number of genes is reduced to less than 50. In certain embodiments, the total number of genes is less than 25. The total number of genes can be reduced to between 15-20, inclusive. In some embodiments the total number of genes are reduced to about 1, 2, 3, 4, 5, 6,7, 8,9, 10, 11, 12, 13, 14, 15, 16, 17, 18, 19, 20, 21, 22, 23, 24, 25, 26, 27, 28, 29, about 30. The method further includes identifying a distinct oncogenic pathway which includes potential therapeutic targets in these various molecular PTCL entities.

In some embodiments, the presently disclosed subject matter provides a method of distinguishing PTCL subtypes using the refined diagnostic and prognostic biomarkers in a subject.

The methods described herein are also useful for selecting treatment for a cancer. For example, an embodiment can comprise providing a cancer sample or obtaining (e.g., isolating) a cancer sample from a subject; measuring at least one of the expression, activity, or product of a gene in the PTCL signature; and selecting a treatment based on the measuring step. Embodiments can further comprise a step of comparing measured levels to those of a control sample or a threshold.

Aspects of the invention also encompasses kits, such as for treating and/or diagnosing cancer.

In an embodiment, the kit can comprise one or more compositions as described herein, such as inhibiting agents, for example.

For example, the kit can comprise one or more reagents useful for detection of the level of a gene in the PTCL signature, or other proteins in a sample. The one or more reagents can be immobilized to a solid support. Non-limiting examples of the composition of the solid support structure comprise plastic, cardboard, glass, plexiglass, tin, paper, or a combination thereof. The solid support can also comprise a dip stick, spoon, scoopula, filter paper or swab.

The reagents can comprise a labeled compound or agent capable of detecting a cancer or tumor cell (e.g., an scFv or monoclonal antibody) in a biological sample; means for determining the amount of gene expression in the sample; and means for comparing the amount of gene expression in the sample with a standard, such as a control sample or threshold. The standard is, in some embodiments, a non-cancer cell or cell extract thereof. The compound or agent can be packaged in a suitable container. The kit can further comprise instructions for using the kit to detect cancer in a sample.

In embodiments, the kit can also include primers for amplifying an mRNA transcribed from a gene that encodes the polypeptide and/or control samples for testing the primers. For example, the control samples can comprise nucleic acids that hybridize to the primers.

The kit can also comprise a sample collection apparatus such as those devices used for collecting a biological fluid or tumor biopsy.

In an embodiment, the kit can include a container that contains the one or more reagents and, optionally (b) informational material. The informational material can be descriptive, instructional, marketing or other material that relates to the methods described herein and/or the use of the agents for diagnostic purposes. In an embodiment, the kit includes also includes one or more anti-cancer therapeutics.

The informational material of the kits is not limited in its form. In one embodiment, the informational material can include information about production of the components of the kit, such as molecular weight, concentration, date of expiration, batch or production site information, and so forth. In one embodiment, the informational material relates to methods of using the components of the kit. The information can be provided in a variety of formats, include printed text, computer readable material, video recording, or audio recording, or information that provides a link or address to substantive material.

The kit can include other ingredients, such as solvents or buffers, a stabilizer, or a preservative. Optionally, the kit can comprise therapeutic agents that can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

The overarching goal of this study is to transition the “academic” PTCL diagnostic and prognostic gene expression (GE) signature originally developed using fresh frozen specimens analyzed with expression microarrays to an optimized, locked-down signature applicable to formalin-fixed paraffin-embedded (FFPE) specimens analyzed on the Nanostring nCounter platform. The two Aims of this proposal—Aim 1. Identify a reduced set of transcripts and create a new model able to replicate the existing “academic” diagnostic and prognostic PTCL signatures; Aim 2. Confirm the applicability of the refined diagnostic and prognostic PTCL gene signature sets on the NanoString nCounter platform for the analysis of FFPE specimens—will determine the feasibility of the optimized FFPE/nCounter assay as a candidate for further commercial test development, which would occur in Phase II studies that would immediately follow the current proposal and include the complete assessment and validation of the analytical and clinical performance, as well as the clinical utility, of the assay.

### Specific Aims

Phase I: Optimization of an “academic” gene expression profiling (GEP) signature for Peripheral T-cell Lymphoma (PTCL) diagnosis and prognosis, and initial assessment of the feasibility of the optimized signature for commercial assay development.

Goal. The overarching goal of this study is to transition an academic PTCL diagnostic/prognostic GEP signature originally developed using fresh frozen (FF) specimens analyzed with expression microarrays to an optimized, locked-down signature applicable to formalin-fixed paraffin-embedded (FFPE) specimens analyzed on the Nanostring nCounter platform. This effort is divided into two Aims and the specific activities for each are detailed below. These Phase I studies will serve to determine the feasibility of the optimized FFPE specimen/nCounter assay as a candidate for further commercial test development (including its offering in CLIA-certified laboratories, and its further development as an in vitro diagnostic (IVD) kit and companion diagnostic (CDx) assay for new or existing PTCL therapies), which will take place—should Phase I objectives be met—in Phase II follow-up studies that would include the complete assessment and validation of the analytical and clinical performance, as well as the clinical utility, of the assay.

Aim 1: Identify a reduced set of transcripts and create a new model able to replicate the existing “academic” diagnostic and prognostic PTCL signatures.

1a. Identify a reduced set of transcripts required for the optimal distinction of each PTCL entity. Deliverables: Identification of those gene transcripts from the original PTCL subtyping signatures that can be transitioned from a fresh frozen (FF) specimen/expression array platform to an FFPE specimen/nCounter platform. These studies will determine the performance of codesets in RNA from approximately 60 FFPE specimens on the nCounter platform compared to data from FF samples from the same cases previously characterized using arrays.

1b. Refine the diagnostic and prognostic signatures that can be applied to FFPE tissues to distinguish PTCL entities. Deliverables: Identification of a single, working minimal gene set that can reproduce the original, “academic” PTCL signatures with an agreement as near unity as possible. Transcripts shown in Aim 1a to transition well from FF/microarrays to the FFPE/nCounter platform will be used in a training set of approximately 120 PTCL cases to develop a classification model and algorithm comprised of the minimal number of genes possible to distinguish among diagnostic and prognostic PTCL subtypes of clinical relevance.

Aim 2: Confirm the applicability of the refined diagnostic and prognostic PTCL gene signature sets on the NanoString nCounter platform for the analysis of FFPE specimens.

2a. Create locked down diagnostic and prognostic PTCL gene signatures and evaluate inter-laboratory reproducibility. Deliverables: Lock-down of the optimized PTCL signature based on an initial validation cohort and inter-laboratory study results. Approximately 100 PTCL FFPE cases will be subtyped using the refined diagnostic and prognostic gene signatures independently in the molecular diagnostic laboratories at UNMC, City of Hope, and HealthChart together with the consulting commercial CLIA lab, Insight Molecular Labs, and the results compared to the FF/microarray gold standard subtyping previously obtained from matched specimens. The inter-lab assay reproducibility will be assessed, the assay algorithm finalized, and the final assay SOP derived.

2b. Validate “locked” diagnostic/prognostic signatures for Phase II assay transition. Deliverables: Validation cohort results confirming the suitability of the locked down signature for advancement into Phase II development. Analysis of a PTCL validation cohort of about 30 FFPE cases will be performed on the nCounter platform in multiple independent laboratories worldwide to confirm the suitability of the optimized subtyping signature, algorithm, and SOP for advancement into Phase II assay development.

### A. Scientific and Clinical Significance

Peripheral T-cell lymphoma (PTCL) is a heterogeneous entity with therapeutic challenges: PTCL constitutes up to 20% of non-Hodgkin's lymphoma^{1,2}. The World Health Organization (WHO) recognizes a number of PTCL subtypes including angioimmunoblastic T-cell lymphoma (AITL), anaplastic large-cell lymphoma (ALCL), adult T-cell leukemia/lymphoma (ATLL), and extra-nodal NK/T-cell lymphoma (ENKTL)³. The diagnosis of PTCL is challenging even for expert hematopathologists⁴ and 30-50% of PTCL are not classifiable with current immunophenotypic and molecular markers and are categorized as PTCL, not otherwise specified (PTCL-NOS)³. With the exception of ALK(+) ALCL (which was characterized by the co-PI, Dr. Steve Morris)⁵ and ALK(−) ALCL with DUSP22 rearrangement, patients with PTCL have a poor prognosis with CHOP-like chemotherapy or more intensive regimens^{4,6,7}. Indeed, the 5-year progression-free survival (PFS) has remained only 20-25% over the last two decades⁸⁻¹². However, new therapies targeted to specific groups of patients have started to be tested, some with spectacular results (see Table 2)¹³⁻¹⁶. Accurate PTCL classification will therefore be essential for both therapeutic trials and in selecting the best treatments for patients in the clinic¹⁷ (e.g., see letters of support from Celgene and Seattle Genetics).

TABLE 2
Phase II studies of new therapeutic agents with
response by subtype (adapted from ref. no. 13).

| | Overall response rate by common subtype | | | |
|---|---|---|---|---|
| | | | | Brentuximab |
| Subtype | Pralatrexate | Romidepsin | Belinostat | vedotin |
| PTCL-NOS | 31% | 29% | 23% | 33% |
| AITL | 8% | 30% | 46% | 54% |
| ALCL | 29% | 24% | 15% | 86% |

Improvement in PTCL diagnosis requires new approaches: We have performed extensive gene expression profiling (GEP) studies and defined robust molecular signatures that can classify the major subtypes of PTCL¹⁰⁻¹². We were also able to re-classify a number of PTCL-NOS instead into distinct entities (15% as AITL, 10% as ALCL, and 9% as γδ-PTCL). Importantly, we also identified two new major subgroups within PTCL-NOS that show enrichment of distinct signaling pathways as well as different prognoses. Further, we identified expression signatures predictive of the survival of AITL patients^{10,11,18}.

Of several platforms tested by the LLMPP for FFPE specimens including Affymetrix¹⁹, NanoString^{20,21}, and quantitative nuclease protection assay (qNPA; HTG Molecular Diagnostics, Inc.)²², the FDA-cleared NanoString nCounter system was superior with respect to specimen handling, lack of a requirement for enzymatic reactions, and direct quantitation of mRNA expression, while also offering high precision and sensitivity²³. Of note, the LLMPP utilized a similar approach as described herein to establish the Lymph2Cx expression-based assay for DLBCL classification on the nCounter platform^{20,21}. This assay was subsequently exclusively licensed by NanoString and is currently being developed in a collaboration with Celgene (worth up to $45 million to NanoString in upfront, development and regulatory milestones, and commercial payments) as an in vitro diagnostic (IVD) companion diagnostic (CDx) test to support the development of lenalidomide as a treatment for patients with DLBCL (http://www.nanostring.com/company/corp_press_release?id=116).

### Innovation

The approach will be practice-changing for the diagnosis, prognostication and management of PTCL by creation of a high-content, quantitative, reproducible molecular assay that will provide greater precision and accuracy of diagnosis of currently defined PTCL subtypes as well as new entities not previously recognizable.

We propose to translate the research diagnostic and prognostic signatures to a platform applicable to formalin-fixed paraffin-embedded (FFPE) tissue and to validate the signatures initially in a CLIA laboratory setting and later as an IVD kit to enable broad clinical usage.

Since new agents in clinical trials show differences in response rates in different PTCL subtypes (e.g., see Table 2), it is critical to evaluate the molecular signatures in experimental trials using a standardized and validated assay. The molecular assay to be developed herein will facilitate the practice of precision medicine for PTCL much like the Lymph2Cx assay is helping to guide the individualized therapy of DLBCL^{20,23}

In summary, we will develop and validate a molecular diagnostic/prognostic assay that reliably identifies clinically relevant PTCL subtypes in routinely available samples, enabling optimal personalized treatment and facilitating trials of new agents by accurate patient stratification.

### B. Preliminary Studies

B.1. Specimen cohort for assay development: We have access to over 1,000 FFPE cases⁴ and over 300 fresh frozen (FF) archival PTCL tissues with pathologic and clinical data¹⁰⁻¹². The FFPE blocks corresponding to the FF samples used in our original GEP studies will be utilized for the initial assay refinement and optimization to be performed herein. We will obtain additional retrospectively collected PTCL samples as needed from multiple European and Asian collaborators (see bios/letters of support) for the initial validation studies to be performed as part of this Phase I proposal, and during the Phase II studies to follow. These tissue and data resources are unparalleled and, combined with cutting-edge technology and sophisticated bioinformatics analysis, will enable us to further optimize the signatures and validate the PTCL assay for clinical applications.

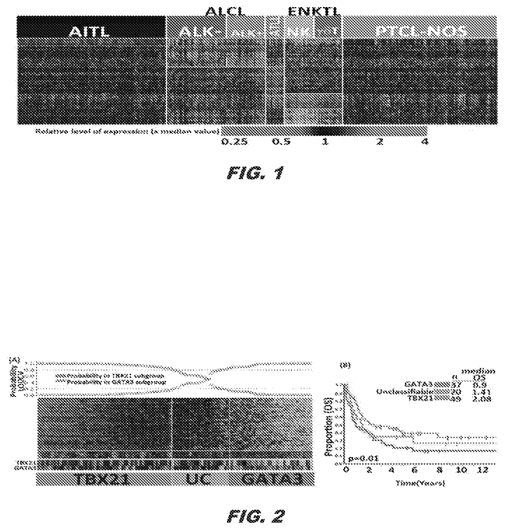

B.2. Robust diagnostic and prognostic signature of PTCL: During development of the academic version of the PTCL assay, Drs. Chan and Iqbal and the extended LLMPP investigators completed gene expression profiling (GEP) on 160 fresh frozen PTCL tissue biopsies using HG U133 plus 2 (U133) microarrays (Affymetrix))^{11,12}. These findings were then refined using a much larger series of cases (n=372)¹⁰. FIG. 1 shows the distinctive gene expression profiles of the major classes of PTCL. This work clearly demonstrated: (a) the successful performance of the diagnostic algorithm based on GEP analysis of fresh frozen tissues from several institutions using a standardized operating procedure, (b) that step-wise binary model algorithms can be used for subgroup classification in complex entities, (c) that the number of genes in a subgroup classifier can be reduced substantially (e.g., from >200 to 22 genes for AITL) without significantly compromising diagnostic accuracy¹⁰, and (d) that distinct oncogenic pathways which include potential therapeutic targets can be identified in these various molecular PTCL entities.

Some additional noteworthy findings included: (i) ALK(−) ALCL is a distinct entity (a finding later supported independently by both genomic and mutational data)²⁴⁻²⁷ and 11% of PTCL-NOS can be reclassified as this entity, which is highly responsive to the anti-CD30 therapy brentuximab vedotin (Table 2). (ii) ENKTL can be subdivided into NK and γδ-T cell subtypes^{10,12}, and ˜9% of PTCL-NOS can be re-classified as γδ-PTCL¹⁰ (a subtype containing activating STAT5B mutations and sensitivity to JAK inhibitors²⁸). Of importance as well, either Aurora kinase A or NOTCH1 inhibition were shown to induce marked cytotoxicity in NK-lymphoma cell lines¹². (iii) AITL prognosis is highly dependent on tumor microenvironment (i.e., the B-cell, dendritic, and monocytic cell content) and the patient quartile with the most favorable signature had a 5-year overall survival (OS) of 55% while the OS of the least favorable quartile was only 15%^{10,11}.

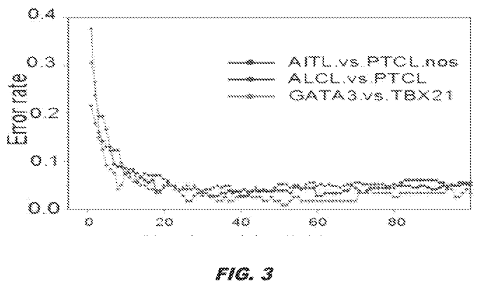

B.3. Identification of distinct subgroups within PTCL-NOS: PTCL-NOS, the largest fraction of PTCL diagnosed with current histopathological methods⁴, can now be separated into two major molecular subtypes characterized by either high expression of the transcription factors GATA3 (˜33%) or TBX21 (˜49%) and many of their target genes¹⁰ (FIG. 2A). Cases with high expression of GATA3 show poorer clinical outcome (FIG. 2B)¹⁰.

B.4. Mutation analysis cross-validates the molecular classification. Our mutational analysis of more than 90 PTCL cases demonstrated the exclusive presence of IDH2^{R172} mutations^{29,30} in GEP-classified or re-classified (from PTCL-NOS) AITL and preferential occurrence of CD28 and RHOA^{G17V} mutations as well³¹. In contrast, JAKI and STAT3 mutations are more prevalent in ALK(−) ALCL²⁶ and STAT5B mutations are frequent in γδ-PTCL²⁸. These observations support the validity of the molecular classification, and these mutations provide independent markers to assess the performance of the molecular classifier.

B.5. GEP signatures in FFPE tissues: Concerns have been raised that FFPE tissues might not yield RNA of sufficient quality for GEP assays. We have shown that GEP signatures obtained from FFPE tissues using expression arrays can robustly subclassify DLBCL¹⁹. In addition, we assessed two non-array platforms, a quantitative nuclease protection assay (HTG Molecular Diagnostics, Inc.)^{22,32} and the nCounter technology^{20,23,33} (NanoString Technologies), and demonstrated the feasibility of measuring signatures in archival DLBCL FFPE specimens. Further, the industry co-PI, Dr. Morris with Brian Z. Ring, PhD, the lead industry bioinformatician on the current proposal, employed RNA-seq to perform GEP of triple-negative breast cancer FFPE specimens to develop the commercially available Insight TNBCtype™ 101-gene assay that identifies 6 molecular TNBC subtypes, each relevant for clinical management³⁴.

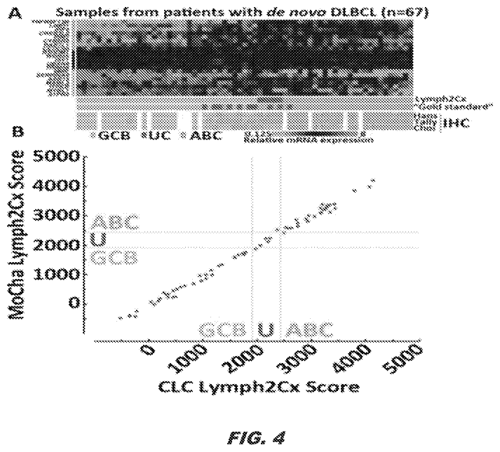

B.6. The gene signature can be refined into a smaller panel: Drs. Chan, Iqbal and coworkers curated 345 genes as the most powerful predictors of PTCL diagnosis and prognosis from their previous array studies¹⁰⁻¹². We are now further refining this gene list to build classifiers with smaller gene numbers. FIG. 3 shows computational modeling demonstrating no significant change in error rate in distinguishing between two PTCL subtypes using smaller panels of 15-20 genes.

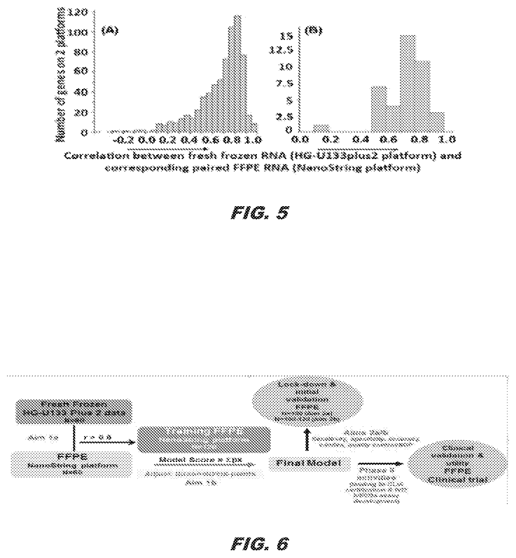

B.7. NanoString Technologies platform for profiling FFPE Tissues: We have shown that a signature derived from FF specimens could be reduced to a 15-gene set on FFPE tissues for DLBCL classification using the NanoString nCounter system: the “Lymph2Cx” signature^{20,23} (FIG. 4A), and this assay could be performed with high reproducibility at multiple labs (FIG. 4B). The Lymph2Cx assay was recently selected as the CDx for FDA evaluation in the current Phase III ROBUST trial (Celgene; ClinicalTrials.gov ID: NCT02285062) examining R-CHOP±lenalidomide for ABC-DLBCL. The development of Lymph2Cx by Drs. Chan, Iqbal and their LLMPP colleagues will be replicated for the assay proposed herein to ensure the creation of standardized and validated clinical diagnostic and prognostic signatures. The likelihood of success is further enhanced by leveraging core capabilities and strengths at the corporate partner, HealthChart LLC, together with our consulting molecular diagnostics firm, Insight Genetics, Inc. (which will participate in the validation study performed in Aim 2a, as described below).

### C. Approach/Specific Aims

Note on technical terminology/statistical considerations: Transcripts are designated “probe sets” on Affymetrix U133 arrays and “codesets” on the NanoString platform throughout the text. The “training set” is comprised of cases from which corresponding GEP data from FF specimens analyzed on U133 arrays is available; the “validation set” is comprised of non-overlapping cases, upon which the locked-down algorithms will be evaluated. The final number of cases to be included in these sets, as well as all other statistical planning and analyses in this project, will be performed by Wu Consulting, Inc., a biostatistics firm that has worked extensively in the pharma and biotech sector since 1993 (www.wuconsulting.com; see letter).

Aim 1: Identify a reduced set of transcripts and create a new model able to replicate the existing “academic” diagnostic and prognostic PTCL signatures.

Overview. The overarching goal of this Phase I STTR proposal is to transition an academic PTCL diagnostic/prognostic GEP signature developed using fresh frozen specimens analyzed with expression arrays to an optimized, locked-down signature applicable to FFPE specimens analyzed with the Nanostring nCounter platform. The current signature¹⁰ is larger than required and would be impractical and uneconomical for clinical assay development. Thus, our initial studies (Aim 1) will reduce the diagnostic sets to the minimum genes possible using the same linear model methodology successfully employed to develop the Lymph2Cx assay for DLBCL²⁰. In addition, however, if needed we will explore complementary approaches (e.g., centroid-based models, as we have used previously)³⁴ to help ensure success in creating a robust model using minimal genes.

Deliverable: Creation of a reduced gene set to be used for the derivation of an optimized, commercially applicable PTCL classification model.

Aim 1a: Identify a reduced set of transcripts required for the optimal distinction of each PTCL entity.

Pre-analytical considerations for FFPE specimens: We have previously compared the performance of 667 diagnostic and prognostic genes identified using U133 arrays for DLBCL against the data obtained using NanoString codesets to analyze FFPE tissues from the same cases (n=88). FIG. 5A shows the correlations between the codesets vs. probe sets in these transcripts. Most of the genes are quite well correlated with a median correlation of 0.75, although some perform very poorly. We specifically examined the correlation of the 40 genes present in both this DLBCL gene set and our academic PTCL signature and observed a correlation of 0.76 between the two platforms (FIG. 5B). We expect a similar correlation will hold true for the rest of the genes in our PTCL signature and, given that only about 15-20 genes should be sufficient for each subtype distinction in a binary model (FIG. 3), we should have an ample number of transcripts from the 345 genes identified in our microarray studies¹⁰⁻¹² to construct a model with a markedly reduced gene number.

Power calculations: The required number of matched FF and FFPE PTCL cases for Aim 1a is relatively small, as these studies seek only to determine the performance of codesets in RNA from FFPE specimens on the nCounter platform compared to data from the corresponding FF samples previously characterized using arrays. We anticipate that 60 samples will be sufficient to bound the correlation of a well-performing transcript having a correlation between the specimen and platform types of 0.80 to be between 0.68 and 0.88 with 95% probability and also bound the correlation of a poorly performing transcript having a correlation of 0.4 to be between 0.155 and 0.60, allowing well- and poor-performing transcripts to be readily distinguished. This sample size will also enable proper scaling and additive adjustment such that for a typical sample the error due to improper adjustment will be <⅕ of the error due to noise between the FF and FFPE specimen modeling.

Aim 1b: Refine the diagnostic and prognostic signatures that can be applied to FFPE tissues to distinguish PTCL entities.

Method for recapitulation of gene signature predictors into an FFPE tissue/NanoString assay: Our original FF RNA PTCL signatures consist of a model score composed of a linear combination of expression values with individual weights based on the discriminative ability of each gene, together with a set of cut-points that divide samples into PTCL molecular subtypes¹⁰⁻¹². We will generate a “training set” with FF PTCL specimens analyzed on U133 arrays and matched FFPE tissues analyzed on the NanoString platform. This training set will accurately estimate the correlation between any given Affymetrix probe sets used in the FF tissue-based model and the corresponding NanoString codesets on FFPE specimens. This analysis will indicate the extent to which the change of platform introduces noise into the expression estimate, and thus the extent to which a specific gene should be down weighted to indicate a reduction in predictive ability in the model or eliminated altogether. Second, once a weighted score has been generated, the training set will be used to estimate the change in centering and dynamic range of the final model score to determine appropriate cut-points. This should be easily accomplished by estimating the mean and SD of the model on the FFPE sample/NanoString assay, and comparing the results to the mean and SD of the FF specimen/array model for matched samples. To meet these goals, the required training set is estimated to be 120 cases (FIG. 6).

Algorithm development for PTCL subtype classification and prognostication: Detailed methodology for subtype classification has been previously described by our team for B-cell lymphomas^{20,23,35-40} and PTCL¹⁰⁻¹². Briefly, for the current studies a series of 4 binary predictors will be used to differentiate four individual subtypes, AITL, ALCL, ATLL and ENKTL, from PTCL-NOS. Based on the results for these predictors we will classify a specimen into one of 6 possible categories (AITL, ALCL, ATLL, ENKTL, PTCL-NOS, or indeterminate) (Table 3). “Indeterminate” samples have features of two or more of the well-defined PTCL subtypes; based on our prior experience¹⁰⁻¹² indeterminate cases should be a very small fraction (1-2%) of samples. PTCL-NOS cases will then be segregated into GATA3- and TBX21-high subgroups, and ENKTL will also be subdivided into NK and gamma/delta T-cell lineages. AITL prognostic subgroups as defined previously¹⁰⁻¹¹ will be translated employing methodology as described for the diagnostic signatures. We will identify well-performing codesets and adjust the weight of each to best reproduce the model scores to divide patients into subgroups that show the greatest survival distinctions. To ensure that the prognostic models are robust we will proceed to lock-down and validation only if they can be demonstrated to differentiate survival with a one-sided p-value of <0.05.

TABLE 3
Stepwise binary classification scheme for subtype analysis.

| Individual binary model results | | | | |
|---|---|---|---|---|
| AITL vs | ALCL vs | ATLL vs | ENKTL vs | Prediction |
| PTCL NOS | PTCL NOS | PTCL NOS | PTCL NOS | Result |
| AITL | PTCL-NOS | PTCL-NOS | PTCL-NOS | AITL |
| PTCL-NOS | ALCL | PTCL-NOS | PTCL-NOS | ALCL |
| PTCL-NOS | PTCL-NOS | ATLL | PTCL-NOS | ATLL |
| PTCL-NOS | PTCL-NOS | PTCL-NOS | ENKTL | ENKTL |
| PTCL-NOS | PTCL-NOS | PTCL-NOS | PTCL-NOS | PTCL-NOS |

| | |
|---|---|
| All other combinations of predictor results | Indeterminate |

Sample size considerations: For this stage of the project we propose collecting additional new samples to raise the total number of paired FF and FFPE specimens to 120 cases (20 AITL, 10 ALK(+) ALCL, 10 ALK(−) ALCL, 20 ATLL, 20 ENKTL, 20 GATA3 PTCL and 20 TBX21 PTCL). With this number of patients we should be able to estimate the noise introduced by the change in platform of a well-measured gene (r>0.8) to within 12% of its true value with 95% confidence. Estimates of the scaling and mean shift necessary to normalize our FFPE model scores to their frozen counterparts, with 95% confidence, suggest the difference between the estimated model thresholds and their optimized value to be less than 20% of the expected noise of an individual sample due to the change in platform. Moreover, 20 samples of each subtype for each binary distinction will also allow us to normalize our model scores to their frozen counterparts, such that the difference between the estimated model cut-points and their optimized value will be, with 95% confidence, only 12% of the variability of an individual sample. Simulations suggest that this degree of inaccuracy will result, with 95% confidence, in at most 4% of the samples being predicted differently from how they would be predicted should a similar NanoString model based on an infinite number of samples be used.

Potential problems and alternative approaches: Re-classification: Discrepant cases will be re-reviewed for morphologic differences between FF and FFPE tissues that can explain the discrepancy^{10,20,23}. The molecular classifications will be cross-validated by analysis of somatic mutations and/or DNA copy number changes unique or enriched in specific subtypes^{10-12,24-31} Clinical outcomes will serve as validation for the prognostic signatures. Classification model: There are a number of additional classification models that will be considered (e.g., centroid-based models we have used previously with success to develop similar expression-based commercial diagnostic assays^{34,41}); however, our PTCL signatures have been tested and validated in FF tissues using linear models and we anticipate robustness of the same model in FFPE tissues, as demonstrated in other studies^{18,42}. Number of genes in model: The current “academic” signature contains 345 genes. Our preliminary bioinformatics studies suggest that a reduction in transcripts to a total of 50-60 or perhaps fewer for the entire signature will be readily doable. Of note, the NanoString platform can assay up to 800 transcripts in a single test.

Specific Aim 2: Confirm the applicability of the refined diagnostic and prognostic PTCL gene signature sets on the NanoString nCounter platform for the analysis of FFPE specimens.

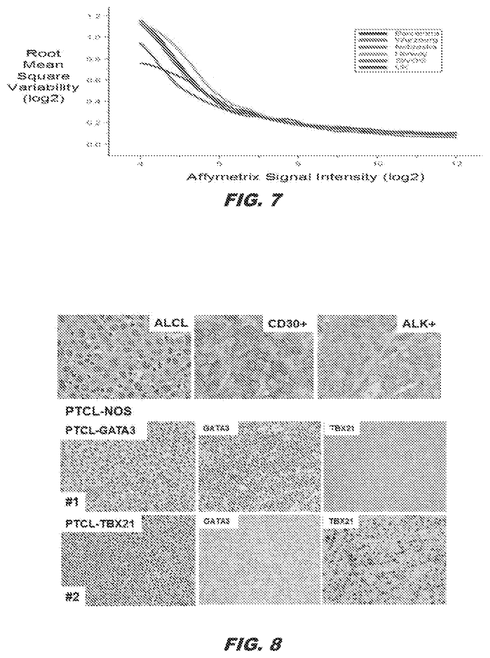

Overview: The overarching goal of Aim 2 will be to demonstrate that the refined diagnostic and prognostic signatures are robust, reproducible, and ready for the transition to Phase II commercial development. The LLMPP consortium has demonstrated low inter-lab variability, during a sample exchange of frozen materials run on an Affymetrix platform (FIG. 7), indicating the ability to develop SOPs and obtain reproducible results across testing sites; herein we will perform similar studies to confirm the reproducibility of our PTCL signatures with FFPE specimens on the NanoString platform in order to advance the assay forward commercially.

Deliverables: Lock-down of the optimized PTCL signature based on training cohort and inter-laboratory study results (Aim 2a); validation cohort results confirming the suitability of the locked down signature for advancement into Phase II development (Aim 2b).

Aim 2a: Create locked down diagnostic and prognostic PTCL gene signatures and evaluate inter-laboratory reproducibility.

We will evaluate our refined diagnostic and prognostic signatures using a non-overlapping series of PTCL cases (˜100) with the same subtype distribution as the training set. Consecutive scrolls from representative FFPE blocks will be evaluated blind using the working “locked down” standard operating procedure (SOP) created from our training set studies (Aim 1b). Assays will be performed in the diagnostic laboratories at UNMC (Dr. Timothy Greiner), City of Hope (Dr. Raju Pillai), and HealthChart with the consulting commercial CLIA lab, Insight Molecular Labs (Drs. Steve Morris and Dave Hout—www.insightgenetics.com; see letter).

Statistical considerations: We will reanalyze a portion of those samples that were part of the initial studies performed by the LLMPP¹⁰⁻¹² (but separate from those cases studied in Aim 1). The relatively small number of specimens (˜100) will not allow us to definitively confirm every single performance parameter of the FFPE model at the statistical metrics desired (due to limitations inherent with Phase I funding). Rather, we will seek to mimic prior LLMPP studies involved in optimizing and transitioning the ABC vs. GCB DLBCL subtyping signature from an FF/array assay to an FFPE/nCounter platform²⁰²¹, in which modest numbers were used for training and validation sets (51 and 68 cases, resp.) to confirm the overall fidelity of subtyping. This approach has been proven successful in follow-up studies of the Lymph2Cx assay: calls were concordant in 96% of 49 repeated biopsies and in 100% of 83 FFPE specimens compared to the gold standard FF/array assay, and critically, no misclassification (ABC- to GCB-DLBCL or vice versa) was observed²¹. To estimate the predictive ability of the FFPE tissue model in PTCL, we will evaluate its performance with a leave-one-out cross validation, including the selection of all weights and thresholds in the model as part of the cross-validation step.

Analysis plan: The FF/microarray diagnosis is considered the gold standard and the accuracy, precision, sensitivity and specificity will be based on comparisons with the array results. The assay results will be provided to Drs. George Wright (NCI Biometric Research Program) and Brian Ring (HealthChart LLC), who will independently determine the classification of the cases. For reproducibility analysis, we will compare the final predicted classifications from the three lab sites as well as the agreement between the model scores relative to their distance from the cut-points to provide a more precise estimate of prediction reproducibility. Based on our prior experience we find that models on NanoString are highly reproducible between sites, generally having a correlation of >0.99^{20,21}. We therefore set a high bar and estimate that 100 samples should be sufficient to achieve this metric with 99% power for each of the models.

Aim 2b: Validate “locked” diagnostic/prognostic signatures for Phase II assay transition.

The goals of Aim 2b are to demonstrate that the locked assay can be readily applied clinically in multiple institutional labs using archival materials and to provide for final troubleshooting, if needed, of the assay to ensure its readiness for Phase II development. A cohort (˜100-120 cases, to include 20 cases of each diagnostic and prognostic subtype) of archival FFPE PTCL specimens will be selected from laboratories worldwide (see letters from collaborating investigators). Since these cases will not have Affymetrix expression array studies performed, direct comparison will not be feasible and the emphasis will be more on the operational aspects of reproducibility, reliability, and problem-solving (if needed) across multiple laboratories using the locked version and SOP. In addition, emphasis will be placed on quality control measures across the different laboratories, which will be noted in order to refine the SOP for future routine testing.

### Example 2

### Plan to Obtain Appropriate Specimens for Developing and Optimizing Assay:

We have assembled ˜70 PTCL cases from our previous GEP study.¹⁰ The pre-selection represents a critical step, as representative tumor sections need to be cut, the presence of representative and adequate tissues confirmed, and the diagnosis of the PTCL cases verified. These cases have gone through a pathology review organized by Drs. Chan and Amador. These included cases, with consensus initial diagnosis and molecular signature diagnosis. Of these, we have consensus diagnosis for AITL (n=18); ALCL (n=15); ENKTL(n=4); PTCL-NOS(n=30). Additional immunostains which are currently used for the characterization or sub-classification were performed on cases, including stains that can help to distinguish between the GATA3 and TBX21 subgroups (FIG. 8). We have developed an immunohistochemistry based algorithm, which can sub-classify PTCL-NOS into GATA3 and TBX21 subgroups. We will continue to refine this IHC algorithm and cross validate the cases with gene signature diagnosis when developed.

Optimization of Isolation of DNA/RNA from Formalin-Fixed Paraffin-Embedded (FFPE) Tissues:

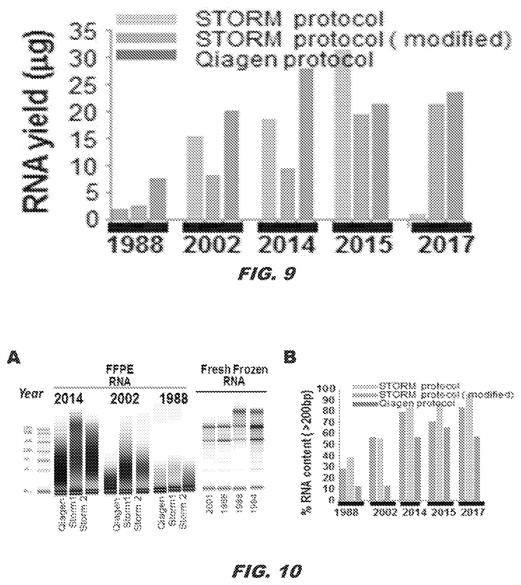

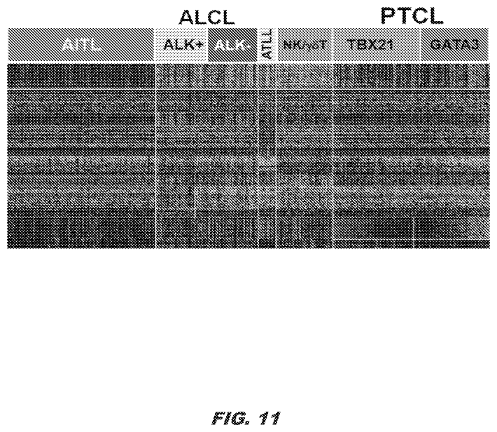

Since the tissues specimens are generally prepared by fixation with formalin (formaldehyde), that leads to biomolecular crosslinks and adducts and reduce signals from molecular analysis involving hybridizations procedures. Generally, isolation of DNA/RNA from FFPTE tissues using commercial kits (e.g. Qiagen AllPrep DNA/RNA FFPE kit) involve heating in Tris buffers at high temperatures (>80° C.) for several hours, that damages RNA and DNA. We have been using these kits routinely for isolation of DNA/RNA for nCounter assays in previous study²⁶ with good success. Since we anticipate a number of FFPE cases from our retrospective series to be older than 10 years and our analytical assay require that at least 40% of total-RNA content within a reaction mixture for nCounter assay should be greater than 200 bp in length. Therefore, we tested a water-soluble bifunctional catalyst (anthranilates and phosphanilates) method that removes formaldehyde adducts from RNA and DNA at lower temperature and shorter incubation time (Karmaker et. al Nat Chem. 2015; 7(9): 752-758) available at https://celldatasci.com/products/RNAstorm/. To test the usefulness of this protocol, we used 10 μm scrolls (2×) of a representative FFPE blocks from 1988 to 2017 (non-essential cases) and compared with Qiagen FFPE isolation kit. The total RNA yield from these kits were in similar range in these cases, but older blocks comparatively showed lower yield (FIG. 9).

The analysis of the size distribution of the RNA molecules using the TapeStation-2200 showed that Strom kits yielded comparatively better (larger size) RNA in older cases (>10 year) than the Qiagen Kit, whereas there was more modest improvement in cases from 2014 FFPE block (FIGS. 10A and 10B). So, the priority will be to use the cases from 2010 onwards for nCounter assays in initial phase. In conclusion, isolation procedure from Qiagen kit can be a feasible method for recent cases (<5 years) whereas if >10 year cases are needed for optimization especially in rare entities like ENKTL, Storm kit isolation can be an effective alternative for isolation of RNA/DNA.

Translation of GEP Data from Affymetrix Probe Sets to nCounter Codeset from NanoString for Multi-Analyte Assays:

Our major objective is to translate the GEP signature from fresh frozen tissues to FFPE tissues using NanoString technologies. Since CodeSet design is expensive and bioinformatically challenging, we refined our gene expression signature from our previous study and added more genes in each diagnostic signature by at least 10% in number, so we have a sufficient number of genes available for refinement of the signature in the event that some of the CodeSets do not work optimally in FFPE RNA without changing the accuracy of the diagnostic signature (FIG. 11). The CodeSets were designed using nDesign™ Gateway, with bioinformatician personnel from NanoString, under an agreement UNeMEd for confidentiality of the gene list. The design includes a total of 416 CodeSets, which include housekeeping genes(n=50), AITL diagnostic (n=50), prognostic (n=49), and PTCL-NOS subgroup (n=50) CodeSets, as well as CodeSets to classify ALCL (including ALK+ALCL vs ALK−ALCL, n=100), to distinguish between NK and the gamma delta sub-group (n=50). We have received these CodeSets for these genes and will begin the assays on FFPE cases in a few weeks. We anticipate that some RNAs will be unsuitable for studies after fixation and need to be excluded, particularly with the smaller signatures to be used. Therefore, we will perform the initial assays on 10 fresh frozen RNA and corresponding FFPET to compare signal strength using the nCounter platform and only transcripts with correlation ≥0.8 will be selected for further evaluation.

The presently disclosed subject matter is further illustrated by the further explanation of the features, benefits, and advantages of the present invention, and by the specific but non-limiting examples as set forth in the Appendix, which are attached hereto and incorporated herein by this reference.

All publications, patents, and patent applications mentioned herein and in the Appendix are incorporated by reference to the same extent as if each individual publication, patent, or patent application was specifically and individually indicated to be incorporated by reference.

It will be understood that various details of the presently disclosed subject matter can be changed without departing from the scope of the subject matter disclosed herein. Furthermore, the foregoing description is for the purpose of illustration only, and not for the purpose of limitation.

### REFERENCES

1. Rudiger T, Weisenburger D D, Anderson J R, et al. Peripheral T-cell lymphoma (excluding anaplastic large-cell lymphoma): results from the Non-Hodgkin's Lymphoma Classification Project. Annals of Oncology: Official journal of the European Society for Medical Oncology ESMO. 2002; 13(1):140-149.
2. Bellei M, Chiattone C S, Luminari S, et al. T-cell lymphomas in South America and Europe. Rev Bras Hematol Hemoter. 2012; 34(1):42-47.
3. Swerdlow S H, Campo E, Harris N L, et al. WHO Classification: Pathology and Genetics of Tumors of Haematopoietic and Lymphoid Tissues. WHO; 4. Lyon, France: IARC Press; 2008.
4. Vose J, Armitage J, Weisenburger D, et al. International Peripheral T-cell and Natural Killer/T-cell Lymphoma Study: pathology findings and clinical outcomes. J Clin Oncol. 2008; 26(25):4124-4130.
5. Morris S W, Kirstein M N, Valentine M B, et al. Fusion of a kinase gene, ALK, to a nucleolar protein gene, NPM, in non-Hodgkin's lymphoma. Science. 1994; 263(5151):1281-1284.
6. Parrilla Castellar E R, Jaffe E S, Said J W, et al. ALK-negative anaplastic large cell lymphoma is a genetically heterogeneous disease with widely disparate clinical outcomes. Blood. 2014; 124(9):1473-1480.
7. Escalon M P, Liu N S, Yang Y, et al. Prognostic factors and treatment of patients with T-cell non-Hodgkin lymphoma: the M. D. Anderson Cancer Center experience. Cancer. 2005; 103(10):2091-2098.
8. Crozier J A, Sher T, Yang D, et al. Persistent disparities among patients with T-cell Non-Hodgkin Lymphomas and B-cell Diffuse Large Cell Lymphomas over 40 years: A SEER database review. Clinical Lymphoma Myeloma and Leukemia. 2015; 15(10):578-85.
9. Xu B, Liu P. No survival improvement for patients with angioimmunoblastic T-cell lymphoma over the past two decades: a population-based study of 1207 cases. PLoS One. 2014; 9(3):e92585.
10. Iqbal J, Wright G, Wang C, et al. Gene expression signatures delineate biological and prognostic subgroups in peripheral T-cell lymphoma. Blood 2014; 123(19):2915-2923.
11. Iqbal J, Weisenburger D D, Greiner T C, et al. Molecular signatures to improve diagnosis in peripheral T-cell lymphoma and prognostication in angioimmunoblastic T-cell lymphoma. Blood 2010; 115(5):1026-1036.
12. Iqbal J, Weisenburger D D, Chowdhury A, et al. Natural killer cell lymphoma shares strikingly similar molecular features with a group of non-hepatosplenic gamma/delta T-cell lymphoma and is highly sensitive to a novel aurora kinase A inhibitor in vitro. Leukemia. 2011; 25(2):348-358.
13. Moskowitz A J, Lunning M A, Horwitz S M. How I treat the peripheral T-cell lymphomas. Blood 2014; 123(17):2636-2644.
14. Gambacorti-Passerini C, Messa C, Pogliani E M. Crizotinib in anaplastic large-cell lymphoma. N Engl J Med 2011; 364(8):775-776.
15. Ishida T, Joh T, Uike N, et al. Defucosylated anti-CCR4 monoclonal antibody (KW-0761) for relapsed adult T-cell leukemia-lymphoma: a multicenter phase II study. J Clin Oncol. 2012; 30(8):837-842.
16. O'Connor O A, Bhagat G, Ganapathi K, et al. Changing the paradigms of treatment in peripheral T-cell lymphoma: from biology to clinical practice. Clin Cancer Res. 2014; 20(20):5240-5254.
17. Coiffier B, Federico M, Caballero D, et al. Therapeutic options in relapsed or refractory peripheral T-cell lymphoma. Cancer Treat Rev. 2014; 40(9):1080-1088.
18. Iqbal J, Wilcox R, Naushad H, et al. Genomic signatures in T-cell lymphoma: How can these improve precision in diagnosis and inform prognosis? Blood Rev. 2016; 30(2):89-100.
19. Linton K, Howarth C, Wappett M, et al. Microarray gene expression analysis of fixed archival tissue permits molecular classification and identification of potential therapeutic targets in diffuse large B-cell lymphoma. J Mol Diagn. 2012; 14(3):223-232.
20. Scott D W, Wright G W, Williams P M, et al. Determining cell-of-origin subtypes of diffuse large B-cell lymphoma using gene expression in formalin-fixed paraffin-embedded tissue. Blood 2014; 123(8):1214-1217.
21. Scott D W, Mottok A, Ennishi D, et al. Prognostic significance of diffuse large B-cell lymphoma cell of origin determined by digital gene expression in formalin-fixed paraffin-embedded tissue biopsies. J Clin Oncol. 2015; 33(26):2848-2856.
22. Rimsza L M, Leblanc M L, Unger J M, et al. Gene expression predicts overall survival in paraffin-embedded tissues of diffuse large B-cell lymphoma treated with R-CHOP. Blood 2008; 112(8):3425-3433.
23. Rimsza L M. Diffuse large B-cell lymphoma classification tied up nicely with a “String”. Clin Cancer Res. 2015; 21(10):2204-2206.
24. Feldman A L, Law M, Remstein E D, et al. Recurrent translocations involving the IRF4 oncogene locus in peripheral T-cell lymphomas. Leukemia. 2009; 23(3):574-580.
25. Feldman A L, Dogan A, Smith D I, et al. Discovery of recurrent t(6;7)(p25.3;q32.3) translocations in ALK-negative anaplastic large cell lymphomas by massively parallel genomic sequencing. Blood 2011; 117(3):915-919.
26. Crescenzo R, Abate F, Lasorsa E, et al. Convergent mutations and kinase fusions lead to oncogenic STAT3 activation in anaplastic large cell lymphoma. Cancer Cell. 2015; 27(4):516-532.
27. Boddicker R L, Kip N S, Xing X, et al. The oncogenic transcription factor IRF4 is regulated by a novel CD30/NF-kappaB positive feedback loop in peripheral T-cell lymphoma. Blood 2015; 125(20):3118-3127.
28. Kucuk C, Jiang B, Hu X, et al. Activating mutations of STAT5B and STAT3 in lymphomas derived from gammadelta-T or N K cells. Nat Commun. 2015; 6:6025.
29. Cairns R A, Iqbal J, Lemonnier F, et al. IDH2 mutations are frequent in angioimmunoblastic T-cell lymphoma. Blood 2012; 119(8):1901-1903.
30. Wang C, McKeithan T W, Gong Q, et al. IDH2^{R172} mutations define a unique subgroup of patients with angioimmunoblastic T-cell lymphoma. Blood 2015; 126(15):1741-1752.
31. Rohr J, Guo S, Huo J, et al. Recurrent activating mutations of CD28 in peripheral T-cell lymphomas. Leukemia. 2016; 30(5):1062-1070.
32. Roberts R A, Sabalos C M, LeBlanc M L, et al. Quantitative nuclease protection assay in paraffin-embedded tissue replicates prognostic microarray gene expression in diffuse large-B-cell lymphoma. Lab Invest. 2007; 87(10):979-997.
33. Geiss G K, Bumgarner R E, Birditt B, et al. Direct multiplexed measurement of gene expression with color-coded probe pairs. Nat Biotechnol. 2008; 26(3):317-325.
34. Ring B Z, Hout D R, Morris S W, et al. Generation of an algorithm based on minimal gene sets to clinically subtype triple negative breast cancer patients. BMC Cancer. 2016 Feb. 23; 16:143.
35. Alizadeh A A, Eisen M B, Davis R E, et al. Distinct types of diffuse large B-cell lymphoma identified by gene expression profiling. Nature. 2000; 403(6769):503-511.
36. Dave S S, Fu K, Wright G W, et al. Molecular diagnosis of Burkitt's lymphoma. N Engl J Med 2006; 354(23):2431-2442.
37. Dave S S, Wright G, Tan B, et al. Prediction of survival in follicular lymphoma based on molecular features of tumor-infiltrating immune cells. N Engl J Med 2004; 351(21):2159-2169.
38. Rosenwald A, Wright G, Chan W C, et al. The use of molecular profiling to predict survival after chemotherapy for diffuse large-B-cell lymphoma. N Engl J Med 2002; 346(25):1937-1947.
39. Rosenwald A, Wright G, Leroy K, et al. Molecular diagnosis of primary mediastinal B cell lymphoma identifies a clinically favorable subgroup of diffuse large B cell lymphoma related to Hodgkin lymphoma. J Exp Med 2003; 198(6):851-862.
40. Rosenwald A, Wright G, Wiestner A, et al. The proliferation gene expression signature is a quantitative integrator of oncogenic events that predicts survival in mantle cell lymphoma. Cancer Cell. February 2003; 3(2):185-197.
41. Tibshirani R, Hastie T, Narasimhan B, Chu G. Diagnosis of multiple cancer types by shrunken centroids of gene expression. Proc Natl Acad Sci USA. 2002; 99(10):6567-6572.
42. Iqbal J, Naushad H, Bi C, et al. Genomic signatures in B-cell lymphoma: How can these improve precision in diagnosis and inform prognosis? Blood Rev. 2016; 30(2):73-88.

### Example 3

### Gene Expression Signatures for the Accurate Diagnosis of Peripheral T-Cell Lymphoma Entities in the Routine Clinical Practice

### Abstract

### Purpose

Peripheral T-cell lymphoma (PTCL) includes heterogeneous clinicopathological entities with numerous diagnostic and treatment challenges. We previously defined robust transcriptomic signatures that distinguish common PTCL entities and identified two novel biological and prognostic PTCL-NOS subtypes (PTCL-TBX21 and PTCL-GATA3). We aimed to consolidate a gene-expression-based subclassification using formalin-fixed, paraffin-embedded tissue (FFPE) tissues to improve the accuracy and precision in PTCL diagnosis.

### Methods

We assembled a well-characterized PTCL training cohort (n=105) with gene expression profiling (GEP) data to derive a diagnostic signature using fresh-frozen (FF) tissue on HG-U133plus2-platform (Affymetrix, Inc) subsequently validated using matched FFPE tissues in a digital GEP platform (NanoString, Inc). Statistical filtering approaches were applied to refine the transcriptomic signatures then validated in another PTCL cohort (n=140) with rigorous pathology review and ancillary assays.

### Results

In the training cohort, the refined transcriptomic classifier in FFPE tissues showed high sensitivity (>80%), specificity (>95%), and accuracy (>94%) for PTCL subclassification compared to the FF-derived diagnostic model and showed high reproducibility between 3 independent laboratories. In the validation cohort, the transcriptional classifier matched the pathology diagnosis rendered by 3-expert hematopathologists in 85% (n=119) of the cases, showed “borderline association” with the molecular signatures in 6% (n=8), and disagreed in 8% (n=11). The classifier improved the pathology diagnosis in 2 cases, validated by clinical findings. Of the 11 cases with disagreements, 4 had a molecular classification that may provide an improvement over pathology diagnosis based on overall transcriptomic and morphological features. The molecular subclassification provided a comprehensive molecular characterization of PTCL subtypes, including viral etiological factors and translocation partners.

### Conclusion

We developed a novel transcriptomic approach for PTCL subclassification that facilitates translation into clinical practice with higher precision and uniformity than conventional pathology diagnosis.

### Context Summary

### Key Objective

To evaluate the role of digital gene-expression signatures for the classification of peripheral T-cell lymphoma (PTCL) using formalin-fixed, paraffin-embedded (FFPE) tissue, and to develop a highly accurate and reproducible diagnostic assay applicable for routine clinical practice.

### Knowledge Generated

Using digital quantitation of transcripts, we have defined robust transcriptomic signatures that can distinguish common PTCL subtypes according to the WHO classification, including two novel biological and prognostic subgroups within PTCL-NOS. We refined the classification algorithm and standardized the assay procedure for a robust diagnostic assay that was validated in an independent PTCL cohort. This assay was reproducible across institutions and showed high classification accuracy.

### Relevance

The digital transcriptomic assay resulted in robust molecular classification of PTCL using mRNA from FFPE tissue, which facilitates translation into a clinical test that provides more precise and uniform diagnosis than conventional pathology. This molecular assay will also facilitate better stratification of cases for research studies and clinical trials.

### Introduction

Peripheral T-cell lymphoma (PTCL) represents ˜10-15% of non-Hodgkin lymphoma (NHL)¹ with numerous challenges in diagnosis even for expert hematopathologists²⁻⁴. The World Health Organization (WHO) classification identifies more than 25 different subtypes of PTCLs, with angioimmunoblastic T-cell lymphoma (AITL), anaplastic large cell lymphoma (ALCL), adult T-cell leukemia/lymphoma (ATLL), and extranodal NK/T-cell lymphoma of nasal type (ENKTCL) as the most frequent entities with geographic variations⁵. However, 30% of PTCL cannot be classified into any of the specific entities in the WHO classification, and these are categorized as PTCL, not otherwise specified (PTCL-NOS)⁶⁷. Tumor-defining abnormalities, such as translocations involving the ALKgene in ALK-positive ALCL (ALK+ALCL)⁸, human T-lymphotropic virus (HTLV-1) infection in ATLL⁹, EBV positivity in ENKTCL, and IDH2^{R172} mutations in AITL, are generally uncommon in PTCL. PTCLs generally have a poor prognosis with current therapies², and more intensive regimens have not been proven superior¹⁰. However, novel targeted therapies are now being tested, with some remarkable results^{11,12}.

The sub-classification is more challenging for PTCL compared to B-cell lymphomas due to the complexity of T-cell biology with numerous functional subsets and functional plasticity. Gene expression profiling (GEP) has aided in delineating novel biological subtypes and in the identification of oncogenic pathways in several B-NHLs¹³⁻¹⁸. Similar approaches in PTCLs resulted in robust molecular classifiers for the common PTCL entities and identified two biological and prognostic subgroups within PTCL-NOS (PTCL-GATA3 and PTCL-TBX21)¹⁹⁻²¹. However, these studies were performed on fresh frozen (FF) samples with transcriptome-wide arrays, thus limiting the application to routine clinical practice^{22,23}. Formalin-fixed, paraffin-embedded (FFPE) tissue samples are widely used in routine diagnosis, but formalin fixation leads to fragmentation, cross-linking, and chemical modifications of RNA and DNA²⁴. Therefore, the effective translation of our highly accurate RNA-based PTCL diagnostic signatures to FFPE tissue is challenging but essential to implement an assay with wide clinical application²⁵. Using digital quantitation of RNA, as in diffuse large B-cell lymphoma²⁶⁻²⁸, we consolidated our PTCL diagnostic signatures from fresh frozen RNA into a single technical platform for the accurate diagnosis across major PTCL entities¹⁹⁻²¹. We used a “training” PTCL cohort (n=105) and validated in an“independent” PTCL cohort (n=140) and among the largest number of well-characterized cases investigated on a single platform for PTCL subclassification. Several viral transcripts were added to improve diagnostic accuracy, and we report a diagnostic algorithm that attained high sensitivity, specificity, and accuracy in distinguishing PTCL entities, including the novel molecular biological subtypes of PTCL-GATA3 and PTCL-TBX21^{20,21}

### Materials and Methods

### Patient Information

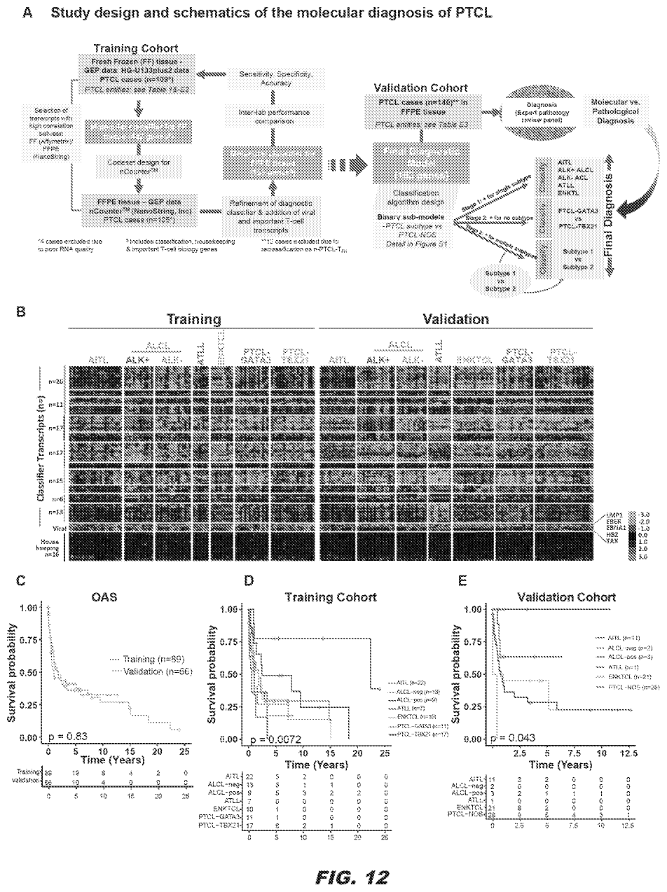

We included 249 diagnostic PTCL cases from multiple institutions, which after the exclusion of 4 cases with poor RNA quality, were divided into a training cohort (105 cases) with previously generated GEP¹⁹⁻²¹ with matched FF and FFPE samples and a validation cohort (140 cases) that had not been previously analyzed (Tables 4 and 5 and FIG. 12A). The basic clinical and pathological characteristics of the cases are shown in Table 6. Inclusion and exclusion criteria of PTCL cases are detailed in the supplemental section.

TABLE 4
Performance of the PTCL diagnostic algorithm in training cohorts.

Training (n = 105)

| Diagnosis | n | Sensitivity | Specificity | Accuracy |
|---|---|---|---|---|
| AITL | 24 | 0.96 | 0.96 | 0.96 |
| ALCL | 29 | 0.93 | 0.99 | 0.97 |
| ALK − ALCL | 15 | 0.87 | 1.00 | 0.98 |
| ALK + ALCL | 14 | 0.93 | 0.98 | 0.97 |
| ENKTCL | 10 | 0.90 | 1.00 | 0.99 |
| ATLL | 7 | 1.00 | 1.00 | 1.00 |
| PTCL-NOS | 35 | 0.86 | 0.96 | 0.92 |
| PTCL-GATA3 | 15 | 0.93 | 0.96 | 0.96 |
| PTCL-TBX21 | 20 | 0.80 | 0.97 | 0.94 |

TABLE 5
Performance of the PTCL diagnostic
algorithm in validation cohorts.

Validation (n = 140)

| Diagnosis | n | Sensitivity | Specificity | Accuracy |
|---|---|---|---|---|
| AITL | 19 | 1.00 | 0.98 | 0.96 |
| ALCL | 36 | 0.89 | 0.99 | 0.96 |
| ALK − ALCL | 16 | 0.75 | 0.99 | 0.96 |
| ALK + ALCL | 20 | 0.90 | 0.98 | 0.97 |
| ENKTCL | 22 | 0.96 | 1.00 | 0.99 |
| ATLL | 12 | 0.83 | 0.98 | 0.97 |
| PTCL-NOS | 51 | 0.94 | 0.93 | 0.94 |
| PTCL-GATA3 | | | | |
| PTCL-TBX21 | | | | |

TABLE 6
Characteristics in training and validation cohort cases and excluded cases.

| | | | | | | Consensus | |
|---|---|---|---|---|---|---|---|
| | | | Follow-up | | HG U133 + 2 | Pathology | Nanostring |
| Case | Cohort | Site | (years) | Status | Classification | (CA, DWD, WCC) | Classification |
| 1 | Training | BCCA | 3.066667 | Alive | AITL | NA | Intermediate. |
| | | (Vancouver BC) | | | | | AITL/GATA3 |
| 2 | Training | NCCS | 7.151667 | Alive | AITL | NA | AITL |
| | | (Singapore) | | | | | |
| 3 | Training | UNMC | 14.96436 | Alive | AITL | NA | AITL |
| 4 | Training | NCCS | 1.12 | Alive | AITL | NA | AITL |
| | | (Singapore) | | | | | |
| 5 | Training | UNMC | 4.662829 | Alive | AITL | NA | AITL |
| 6 | Training | NCCS | 1.231667 | Alive | AITL | NA | AITL |
| | | (Singapore) | | | | | |
| 7 | Training | NCCS | 1.12 | Alive | AITL | NA | AITL |
| | | (Singapore) | | | | | |
| 8 | Training | BCCA | 0.775 | Dead | AITL | NA | Intermediate |
| | | (Vancouver BC) | | | | | AITL/TBX21 |
| 9 | Training | UNMC | 14.79441 | Dead | AITL | NA | PTCL-GATA3 |
| 10 | Training | BCCA | 0.252778 | Dead | AITL | NA | AITL |
| | | (Vancouver BC) | | | | | |
| 11 | Training | NCCS | 1.806667 | Dead | AITL | NA | AITL |
| | | (Singapore) | | | | | |
| 12 | Training | UNMC | 0.055833 | Dead | AITL | NA | AITL |
| 13 | Training | UNMC | 2.9 | Dead | AITL | NA | AITL |
| 14 | Training | UNMC | 0.005482 | Dead | AITL | NA | AITL |
| 15 | Training | NCCS | 2.239167 | Dead | AITL | NA | AITL |
| | | (Singapore) | | | | | |
| 16 | Training | BCCA | 0.322222 | Dead | AITL | NA | AITL |
| | | (Vancouver BC) | | | | | |
| 17 | Training | BCCA | 2.25 | Dead | AITL | NA | AITL |
| | | (Vancouver BC) | | | | | |
| 18 | Training | NCCS | 0.076667 | Dead | AITL | NA | AITL |
| | | (Singapore) | | | | | |
| 19 | Training | NCCS | 1.2375 | Dead | AITL | NA | AITL |
| | | (Singapore) | | | | | |
| 20 | Training | NCCS | 0.180833 | Dead | AITL | NA | AITL |
| | | (Singapore) | | | | | |
| 21 | Training | NCCS | 0.4025 | Dead | AITL | NA | AITL |
| | | (Singapore) | | | | | |
| 22 | Training | BCCA | 0.622222 | Dead | AITL | NA | AITL |
| | | (Vancouver BC) | | | | | |
| 23 | Training | Mayo Clinic | NA | NA | AITL | NA | Intermediate |
| | | (Rochester) | | | | | AITL/TBX21 |
| 24 | Training | Mayo Clinic | NA | NA | AITL | NA | AITL |
| | | (Rochester) | | | | | |
| 25 | Training | NCCS | 2.76 | Alive | ALK− ALCL | NA | ALK− ALCL |
| | | (Singapore) | | | | | |
| 26 | Training | UNMC | 5.75 | Alive | ALK− ALCL | NA | ALK− ALCL |
| 27 | Training | NCCS | 0.3425 | Alive | ALK− ALCL | NA | ALK− ALCL |
| | | (Singapore) | | | | | |
| 28 | Training | BCCA | 2.475 | Dead | ALK− ALCL | NA | PTCL-TBX21 |
| | | (Vancouver BC) | | | | | |
| 29 | Training | NCCS | 0.0875 | Dead | ALK− ALCL | NA | ALK− ALCL |
| | | (Singapore) | | | | | |
| 30 | Training | BCCA | 0.088889 | Dead | ALK− ALCL | NA | ALK− ALCL |
| | | (Vancouver BC) | | | | | |
| 31 | Training | BCCA | 0.030556 | Dead | ALK− ALCL | NA | ALK− ALCL |
| | | (Vancouver BC) | | | | | |
| 32 | Training | BCCA | 0.094444 | Dead | ALK− ALCL | NA | ALK− ALCL |
| | | (Vancouver BC) | | | | | |
| 33 | Training | International | 0.11499 | Dead | ALK− ALCL | NA | ALK− ALCL |
| | | PTCL Study | | | | | |
| 34 | Training | NCCS | 7.22 | Dead | ALK− ALCL | NA | ALK+ ALCL |
| | | (Singapore) | | | | | |
| 35 | Training | International | 1.820671 | Dead | ALK− ALCL | NA | ALK− ALCL |
| | | PTCL Study | | | | | |
| 36 | Training | UNMC | 0.844333 | Dead | ALK− ALCL | NA | ALK− ALCL |
| 37 | Training | NCCS | 15.09083 | Dead | ALK− ALCL | NA | ALK+ ALCL |
| | | (Singapore) | | | | | |
| 38 | Training | NCCS | NA | NA | ALK− ALCL | NA | ALK− ALCL |
| | | (Singapore) | | | | | |
| 39 | Training | NCCS | NA | NA | ALK− ALCL | NA | ALK− ALCL |
| | | (Singapore) | | | | | |
| 40 | Training | NCCS | NA | NA | ALK− ALCL | NA | ALK− ALCL |
| | | (Singapore) | | | | | |
| 41 | Training | UNMC | 5.5 | Alive | ALK+ ALCL | NA | ALK+ ALCL |
| 42 | Training | UNMC | 13.665 | Alive | ALK+ ALCL | NA | ALK+ ALCL |
| 43 | Training | NCCS | 5.741667 | Alive | ALK+ ALCL | NA | ALK+ ALCL |
| | | (Singapore) | | | | | |
| 44 | Training | UNMC | 24.07617 | Alive | ALK+ ALCL | NA | ALK+ ALCL |
| 45 | Training | BCCA | 4.836111 | Alive | ALK+ ALCL | NA | ALK+ ALCL |
| | | (Vancouver BC) | | | | | |
| 46 | Training | BCCA | 4.277778 | Alive | ALK+ ALCL | NA | ALK+ ALCL |
| | | (Vancouver BC) | | | | | |
| 47 | Training | NCCS | 0.898333 | Dead | ALK+ ALCL | NA | ALK+ ALCL |
| | | (Singapore) | | | | | |
| 48 | Training | NCCS | 0.990833 | Dead | ALK+ ALCL | NA | ALK+ ALCL |
| | | (Singapore) | | | | | |
| 49 | Training | UNMC | 22.35742 | Dead | ALK+ ALCL | NA | AITL |
| 50 | Training | Mayo Clinic | NA | NA | ALK+ ALCL | NA | ALK+ ALCL |
| | | Rochester) | | | | | |
| 51 | Training | NCCS | NA | NA | ALK+ ALCL | NA | ALK+ ALCL |
| | | (Singapore) | | | | | |
| 52 | Training | Mayo Clinic | NA | NA | ALK+ ALCL | NA | ALK+ ALCL |
| | | (Rochester) | | | | | |
| 53 | Training | Mayo Clinic | NA | NA | ALK+ ALCL | NA | ALK+ ALCL |
| | | (Rochester) | | | | | |
| 54 | Training | International | 0.580424 | Alive | ATLL | NA | ATLL |
| | | PTCL Study | | | | | |
| 55 | Training | International | 3.425051 | Dead | ATLL | NA | ATLL |
| | | PTCL Study | | | | | |
| 56 | Training | International | 0.99384 | Dead | ATLL | NA | ATLL |
| | | PTCL Study | | | | | |
| 57 | Training | International | 0.741958 | Dea | ATLL | NA | ATLL |
| | | PTCL Study | | | | | |
| 58 | Training | International | 0.334018 | Dead | ATLL | NA | ATLL |
| | | PTCL Study | | | | | |
| 59 | Training | International | 1.281314 | Dead | ATLL | NA | ATLL |
| | | PTCL Study | | | | | |
| 60 | Training | International | 0.969199 | Dead | ATLL | NA | ATLL |
| | | PTCL Study | | | | | |
| 61 | Training | Sungkyunkwan | 0.05 | Alive | NA | ENKTL | ENKTL |
| | | University | | | | | |
| | | (Korea) | | | | | |
| 62 | Training | Sungkyunkwan | 3.675 | Alive | NA | ENKTL | ENKTL |
| | | University | | | | | |
| | | (Korea) | | | | | |
| 63 | Training | NCCS | 0.870833 | Alive | ENKTL | NA | ENKTL |
| | | (Singapore) | | | | | |
| 64 | Training | NCCS | 7.293333 | Alive | ENKTL | NA | ENKTL |
| | | (Singapore) | | | | | |
| 65 | Training | UNMC | 0.038417 | Dead | ENKTL | NA | PTCL-TBX21 |
| 66 | Training | Sungkyunkwan | 0.708333 | Dead | NA | ENKTL | ENKTL |
| | | University | | | | | |
| | | (Korea) | | | | | |
| 67 | Training | Sungkyunkwan | 1.591667 | Dead | NA | ENKTL | ENKTL |
| | | University | | | | | |
| | | (Korea) | | | | | |
| 68 | Training | Sungkyunkwan | 1.008333 | Dead | NA | ENKTL | ENKTL |
| | | University | | | | | |
| | | (Korea) | | | | | |
| 69 | Training | NCCS | 1.141667 | Dead | ENKTL | NA | ENKTL |
| | | (Singapore) | | | | | |
| 70 | Training | International | 0.197125 | Dead | ENKTL | NA | ENKTL |
| | | PTCL Study | | | | | |
| 71 | Training | Wurzburg/ | 7.972222 | Alive | PTCL-GATA3 | NA | PTCL-GATA3 |
| | | Stuttgart | | | | | |
| 72 | Training | NCCS | 0.72 | Alive | PTCL-GATA3 | NA | PTCL-GATA3 |
| | | (Singapore) | | | | | |
| 73 | Training | NCCS | 3.033333 | Alive | PTCL-GATA3 | NA | Intermediate.AIT |
| | | (Singapore) | | | | | L/GATA3 |
| 74 | Training | UNMC | 0.589167 | Dead | PTCL-GATA3 | NA | PTCL-GATA3 |
| 75 | Training | NCCS | 3.414167 | Dead | PTCL-GATA3 | NA | PTCL-GATA3 |
| | | (Singapore) | | | | | |
| 76 | Training | UNMC | 0.566667 | Dead | PTCL-GATA3 | NA | PTCL-GATA3 |
| 77 | Training | UNMC | 0.046667 | Dead | PTCL-GATA3 | NA | PTCL-GATA3 |
| 78 | Training | Wurzburg/ | 0.075 | Dead | PTCL-GATA3 | NA | PTCL-GATA3 |
| | | Stuttgart | | | | | |
| 79 | Training | NCCS | 0.194167 | Dead | PTCL-GATA3 | NA | PTCL-GATA3 |
| | | (Singapore) | | | | | |
| 80 | Training | UNMC | 0.078333 | Dead | PTCL-GATA3 | NA | PTCL-GATA3 |
| 81 | Training | NCCS | 0.355833 | Dead | PTCL-GATA3 | NA | Intermediate. |
| | | (Singapore) | | | | | AITL/GATA3 |
| 82 | Training | Mayo Clinic | NA | NA | PTCL-GATA3 | NA | PTCL-GATA3 |
| | | (Rochester) | | | | | |
| 83 | Training | Wurzburg/ | NA | NA | PTCL-GATA3 | NA | PTCL-GATA3 |
| | | Stuttgart | | | | | |
| 84 | Training | Mayo Clinic | NA | NA | PTCL-GATA3 | NA | ALK+ ALCL |
| | | (Rochester) | | | | | |
| 85 | Training | Mayo Clinic | NA | NA | PTCL-GATA3 | NA | PTCL-GATA3 |
| | | (Rochester) | | | | | |
| 86 | Training | NCCS | 0.815833 | Alive | PTCL-TBX21 | NA | PTCL-TBX21 |
| | | (Singapore) | | | | | |
| 87 | Training | UNMC | 13.04583 | Alive | PTCL-TBX21 | NA | PTCL-TBX21 |
| 88 | Training | Wurzburg/ | 0.632444 | Alive | PTCL-TBX21 | NA | PTCL-TBX21 |
| | | Stuttgart | | | | | |
| 89 | Training | Wurzburg/ | 1.369444 | Alive | PTCL-TBX21 | NA | Intermediate |
| | | Stuttgart | | | | | AITL/TBX21 |
| 90 | Training | BCCA | 5.563889 | Alive | PTCL-TBX21 | NA | PTCL-TBX21 |
| | | (Vancouver BC) | | | | | |
| 91 | Training | NCCS | 0.0025 | Alive | PTCL-TBX21 | NA | Intermediate |
| | | (Singapore) | | | | | AITL/TBX21 |
| 92 | Training | UNMC | 6.09863 | Alive | PTCL-TBX21 | NA | AITL |
| 93 | Training | BCCA | 7.922222 | Dead | PTCL-TBX21 | NA | AITL |
| | | (Vancouver BC) | | | | | |
| 94 | Training | UNMC | 2.319167 | Dead | PTCL-TBX21 | NA | PTCL-GATA3 |
| 95 | Training | BCCA | 2.452778 | Dead | PTCL-TBX21 | NA | PTCL-GATA3 |
| | | (Vancouver BC) | | | | | |
| 96 | Training | UNMC | 9.498333 | Dead | PTCL-TBX21 | NA | PTCL-TBX21 |
| 97 | Training | UNMC | 1.416667 | Dead | PTCL-TBX21 | NA | PTCL-TBX21 |
| 98 | Training | NCCS | 0.051667 | Dead | PTCL-TBX21 | NA | Intermediate |
| | | (Singapore) | | | | | AITL/TBX21 |
| 99 | Training | BCCA | 0.975 | Dead | PTCL-TBX21 | NA | Intermediate |
| | | (Vancouver BC) | | | | | AITL/TBX21 |
| 100 | Training | NCCS | 0.164167 | Dead | PTCL-TBX21 | NA | Intermediate |
| | | (Singapore) | | | | | AITL/TBX21 |
| 101 | Training | BCCA | 18.39167 | Dead | PTCL-TBX21 | NA | PTCL-TBX21 |
| | | (Vancouver BC) | | | | | |
| 102 | Training | Wurzburg/ | 0.516667 | Dead | PTCL-TBX21 | NA | PTCL-TBX21 |
| | | Stuttgart | | | | | |
| 103 | Training | Mayo Clinic | NA | NA | PTCL-TBX21 | NA | PTCL-TBX21 |
| | | (Rochester) | | | | | |
| 104 | Training | Wurzburg/ | NA | NA | PTCL-TBX21 | NA | PTCL-TBX21 |
| | | Stuttgart | | | | | |
| 105 | Training | NCCS | NA | NA | PTCL-TBX21 | NA | PTCL-TBX21 |
| | | (Singapore) | | | | | |
| 106 | Validation | Wurzburg/ | 0.519444 | Alive | NA | AITL | AITL |
| | | Stuttgart | | | | | |
| 107 | Validation | Wurzburg/ | 0.005556 | Alive | NA | AITL | AITL |
| | | Stuttgart | | | | | |
| 108 | Validation | Wurzburg/ | 3.833333 | Alive | NA | AITL | AITL |
| | | Stuttgart | | | | | |
| 109 | Validation | Wurzburg/ | 6.338889 | Alive | NA | AITL | AITL |
| | | Stuttgart | | | | | |
| 110 | Validation | Wurzburg/ | 0.2 | Alive | NA | AITL | AITL |
| | | Stuttgart | | | | | |
| 111 | Validation | Wurzburg/ | 1.019444 | Alive | NA | AITL | Intermediate |
| | | Stuttgart | | | | | AITL/GATA3 |
| 112 | Validation | Wurzburg/ | 0.747222 | Alive | NA | AITL | AITL |
| | | Stuttgart | | | | | |
| 113 | Validation | UNMC | 5.605833 | Alive | NA | AITL | Intermediate |
| | | | | | | | AITL/TBX21 |
| 114 | Validation | UNMC | 0.578417 | Dead | NA | AITL | AITL |
| 115 | Validation | OHSU | 0.670773 | Dead | NA | AITL | AITL |
| 116 | Validation | Wurzburg/ | 0.438889 | Dead | NA | AITL | AITL |
| | | Stuttgart | | | | | |
| 117 | Validation | BCCA | NA | NA | NA | AITL | AITL |
| | | (Vancouver BC) | | | | | |
| 118 | Validation | Wurzburg/ | NA | NA | NA | AITL | Intermediate |
| | | Stuttgart | | | | | AITL/GATA3 |
| 119 | Validation | Wurzburg/ | NA | NA | NA | AITL | AITL |
| | | Stuttgart | | | | | |
| 120 | Validation | Wurzburg/ | NA | NA | NA | AITL | Intermediate |
| | | Stuttgart | | | | | AITL/TBX21 |
| 121 | Validation | Cleveland | NA | NA | NA | AITL | AITL |
| | | Clinic | | | | | |
| 122 | Validation | UNMC | NA | NA | NA | AITL | AITL |
| 123 | Validation | UNMC | NA | NA | NA | AITL | AITL |
| 124 | Validation | UNMC | NA | NA | NA | AITL | Intermediate |
| | | | | | | | AITL/TBX21 |
| 125 | Validation | City of Hope | NA | NA | NA | ALK− ALCL | ALK− ALCL |
| 126 | Validation | City of Hope | NA | NA | NA | ALK− ALCL | ALK− ALCL |
| 127 | Validation | UNMC | 0.016417 | Dead | NA | ALK− ALCL | ALK− ALCL |
| 128 | Validation | UNMC | 0.392 | Dead | NA | ALK− ALCL | PTCL-TBX21 |
| 129 | Validation | Milan/Bologna | NA | NA | NA | ALK− ALCL | ALK− ALCL |
| | | University | | | | | |
| | | (Italy) | | | | | |
| 130 | Validation | Milan/Bologna | NA | NA | NA | ALK− ALCL | ALK+ ALCL |
| | | University | | | | | |
| | | (Italy) | | | | | |
| 131 | Validation | BCCA | NA | NA | NA | ALK− ALCL | ALK− ALCL |
| | | (Vancouver BC) | | | | | |
| 132 | Validation | BCCA | NA | NA | NA | ALK− ALCL | ALK− ALCL |
| | | (Vancouver BC) | | | | | |
| 133 | Validation | City of Hope | NA | NA | NA | ALK− ALCL | ALK− ALCL |
| 134 | Validation | City of Hope | NA | NA | NA | ALK− ALCL | ALK+ ALCL |
| 135 | Validation | UNMC | NA | NA | NA | ALK− ALCL | ALK− ALCL |
| 136 | Validation | UNMC | NA | NA | NA | ALK− ALCL | ALK− ALCL |
| 137 | Validation | UNMC | NA | NA | NA | ALK− ALCL | ALK− ALCL |
| 138 | Validation | UNMC | NA | NA | NA | ALK− ALCL | ALK− ALCL |
| 139 | Validation | UNMC | NA | NA | NA | ALK− ALCL | ALK− ALCL |
| 140 | Validation | UNMC | NA | NA | NA | ALK− ALCL | PTCL-TBX21 |
| 141 | Validation | UNMC | 0.515333 | Alive | NA | ALK+ ALCL | ALK+ ALCL |
| 142 | Validation | UNMC | 3.157917 | Alive | NA | ALK+ ALCL | ALK+ ALCL |
| 143 | Validation | UNMC | 10.72917 | Alive | NA | ALK+ ALCL | ALK+ ALCL |
| 144 | Validation | City of Hope | NA | NA | NA | ALK+ ALCL | PTCL-GATA3 |
| 145 | Validation | Milan/Bologna | NA | NA | NA | ALK+ ALCL | ALK+ ALCL |
| | | University | | | | ALK+ ALCL | ALK+ ALCL |
| | | (Italy) | | | | | |
| 146 | Validation | Milan/Bologna | NA | NA | NA | | |
| | | University | | | | | |
| | | (Italy) | | | | | |
| 147 | Validation | Milan/Bologna | NA | NA | NA | ALK+ ALCL | ALK+ ALCL |
| | | University | | | | | |
| | | (Italy) | | | | | |
| 148 | Validation | Milan/Bologna | NA | NA | NA | ALK+ ALCL | ALK+ ALCL |
| | | University | | | | | |
| | | (Italy) | | | | | |
| 149 | Validation | City of Hope | NA | NA | NA | ALK+ ALCL | ALK+ ALCL |
| 150 | Validation | BCCA | NA | NA | NA | ALK+ ALCL | ALK+ ALCL |
| | | (Vancouver BC) | | | | | |
| 151 | Validation | City of Hope | NA | NA | NA | ALK+ ALCL | ALK+ ALCL |
| 152 | Validation | City of Hope | NA | NA | NA | ALK+ ALCL | ALK+ ALCL |
| 153 | Validation | City of Hope | NA | NA | NA | ALK+ ALCL | ALK+ ALCL |
| 154 | Validation | City of Hope | NA | NA | NA | ALK+ ALCL | ALK+ ALCL |
| 155 | Validation | UNMC | NA | NA | NA | ALK+ ALCL | ALK+ ALCL |
| 156 | Validation | UNMC | NA | NA | NA | ALK+ ALCL | ALK+ ALCL |
| 157 | Validation | UNMC | NA | NA | NA | ALK+ ALCL | ALK+ ALCL |
| 158 | Validation | UNMC | NA | NA | NA | ALK+ ALCL | PTCL-TBX21 |
| 159 | Validation | City of Hope | NA | NA | NA | ALK, ALK pos | ALK+ ALCL |
| 160 | Validation | Milan/Bologna | NA | NA | NA | ALK, ALK pos | ALK+ ALCL |
| | | University | | | | | |
| | | (Italy) | | | | | |
| 161 | Validation | UNMC | 1.102 | Alive | NA | ATLL | PTCL-GATA3 |
| 162 | Validation | Weill-Cornell | NA | NA | NA | ATLL | PTCL-GATA3 |
| 163 | Validation | City of Hope | NA | NA | NA | ATLL | ATLL |
| 164 | Validation | University | NA | NA | NA | ATLL | ATLL |
| | | of Miami | | | | | |
| 165 | Validation | INEN (Peru) | NA | NA | NA | ATLL | ATLL |
| 166 | Validation | INEN (Peru) | NA | NA | NA | ATLL | ATLL |
| 167 | Validation | INEN (Peru) | NA | NA | NA | ATLL | ATLL |
| 168 | Validation | INEN (Peru) | NA | NA | NA | ATLL | ATLL |
| 169 | Validation | Weill-Cornell | NA | NA | NA | ATLL | ATLL |
| 170 | Validation | Weill-Cornell | NA | NA | NA | ATLL | ATLL |
| 171 | Validation | Weill-Cornell | NA | NA | NA | ATLL | ATLL |
| 172 | Validation | UNMC | NA | NA | NA | ATLL | ATLL |
| 173 | Validation | City of Hope | 4.679452 | Alive | NA | ENKTL | ENKTL |
| 174 | Validation | City of Hope | 3.430137 | Alive | NA | ENKTL | ENKTL |
| 175 | Validation | City of Hope | 1.00274 | Alive | NA | ENKTL | ENKTL |
| 176 | Validation | City of Hope | 4.512329 | Alive | NA | ENKTL | ENKTL |
| 177 | Validation | City of Hope | 4.59726 | Alive | NA | ENKTL | ALK− ALCL |
| 178 | Validation | Sungkyunkwan | 6.391667 | Alive | NA | ENKTL | ENKTL |
| | | University | | | | | |
| | | (Korea) | | | | | |
| 179 | Validation | Sungkyunkwan | 4.275 | Alive | NA | ENKTL | ENKTL |
| | | University | | | | | |
| | | (Korea) | | | | | |
| 180 | Validation | Sungkyunkwan | 3.433333 | Alive | NA | ENKTL | ENKTL |
| | | University | | | | | |
| | | (Korea) | | | | | |
| 181 | Validation | Sungkyunkwan | 0.133333 | Alive | NA | ENKTL | ENKTL |
| | | University | | | | | |
| | | (Korea) | | | | | |
| 182 | Validation | BCCA | 5.136208 | Dead | NA | ENKTL | ENKTL |
| | | (Vancouver BC) | | | | | |
| 183 | Validation | UNMC | 0.276864 | Dead | NA | ENKTL | ENKTL |
| 184 | Validation | City of Hope | 0.671233 | Dead | NA | ENKTL | ENKTL |
| 185 | Validation | City of Hope | 0.457534 | Dead | NA | ENKTL | ENKTL |
| 186 | Validation | City of Hope | 0.167123 | Dead | NA | ENKTL | ENKTL |
| 187 | Validation | City of Hope | 0.331507 | Dead | NA | ENKTL | ENKTL |
| 188 | Validation | Sungkyunkwan | 0.175 | Dead | NA | ENKTL | ENKTL |
| | | University | | | | | |
| | | (Korea) | | | | | |
| 189 | Validation | Sungkyunkwan | 0.575 | Dead | NA | ENKTL | ENKTL |
| | | University | | | | | |
| | | (Korea) | | | | | |
| 190 | Validation | Sungkyunkwan | 0 | Dead | NA | ENKTL | ENKTL |
| | | University | | | | | |
| | | (Korea) | | | | | |
| 191 | Validation | Sungkyunkwan | 0.075 | Dead | NA | ENKTL | ENKTL |
| | | University | | | | | |
| | | (Korea) | | | | | |
| 192 | Validation | Sungkyunkwan | 0.975 | Dead | NA | ENKTL | ENKTL |
| | | University | | | | | |
| | | (Korea) | | | | | |
| 193 | Validation | Sungkyunkwan | 0.441667 | Dead | NA | ENKTL | ENKTL |
| | | University | | | | | |
| | | (Korea) | | | | | |
| 194 | Validation | City of Hope | NA | NA | NA | ENKTL | ENKTL |
| 195 | Validation | Cleveland | 3.455556 | Alive | NA | PTCL-NOS | PTCL-GATA3 |
| | | Clinic | | | | | |
| 196 | Validation | Cleveland | 12.68611 | Alive | NA | PTCL-NOS | PTCL-GATA3 |
| | | Clinic | | | | | |
| 197 | Validation | BCCA | 8.971937 | Alive | NA | PTCL-NOS | AITL |
| | | (Vancouver BC) | | | | | |
| 198 | Validation | City of Hope | 0.011111 | Alive | NA | PTCL-NOS | PTCL-TBX21 |
| 199 | Validation | OHSU | 3.578371 | Alive | NA | PTCL-NOS | PTCL-TBX21 |
| 200 | Validation | Wurzburg/ | 0.083333 | Alive | NA | PTCL-NOS | PTCL-TBX21 |
| | | Stuttgart | | | | | |
| 201 | Validation | UNMC | 10.94298 | Alive | NA | PTCL-NOS | PTCL-TBX21 |
| 202 | Validation | UNMC | 11.46107 | Alive | NA | PTCL-NOS | PTCL-TBX21 |
| 203 | Validation | Wurzburg/ | 0.061111 | Alive | NA | PTCL-NOS | PTCL-GATA3 |
| | | Stuttgart | | | | | |
| 204 | Validation | Wurzburg/ | 0.086111 | Dead | NA | PTCL-NOS | PTCL-GATA3 |
| | | Stuttgart | | | | | |
| 205 | Validation | BCCA | 0.396988 | Dead | NA | PTCL-NOS | Intermediate |
| | | (Vancouver BC) | | | | | AITL/GATA3 |
| 206 | Validation | BCCA | 0.577687 | Dead | NA | PTCL-NOS | PTCL-GATA3 |
| | | (Vancouver BC) | | | | | |
| 207 | Validation | OHSU | 0.462697 | Dead | NA | PTCL-NOS | PTCL-GATA3 |
| 208 | Validation | BCCA | 5.900068 | Dead | NA | PTCL-NOS | PTCL-TBX21 |
| | | (Vancouver BC) | | | | | |
| 209 | Validation | BCCA | 1.078713 | Dead | NA | PTCL-NOS | PTCL-TBX21 |
| | | (Vancouver BC) | | | | | |
| 210 | Validation | BCCA | 3.206023 | Dead | NA | PTCL-NOS | PTCL-TBX21 |
| | | (Vancouver BC) | | | | | |
| 211 | Validation | Wurzburg/ | 0 | Dead | NA | PTCL-NOS | PTCL-TBX21 |
| | | Stuttgart | | | | | |
| 212 | Validation | Cleveland | 0.166667 | Dead | NA | PTCL-NOS | PTCL-TBX21 |
| | | Clinic | | | | | |
| 213 | Validation | Cleveland | 2 | Dead | NA | PTCL-NOS | PTCL-TBX21 |
| | | Clinic | | | | | |
| 214 | Validation | BCCA | 0.112252 | Dead | NA | PTCL-NOS | AITL |
| | | (Vancouver BC) | | | | | |
| 215 | Validation | BCCA | 0.498289 | Dead | NA | PTCL-NOS | PTCL-TBX21 |
| | | (Vancouver BC) | | | | | |
| 216 | Validation | BCCA | 0.982888 | Dead | NA | PTCL-NOS | PTCL-TBX21 |
| | | (Vancouver BC) | | | | | |
| 217 | Validation | OHSU | 0.574949 | Dead | NA | PTCL-NOS | PTCL-TBX21 |
| 218 | Validation | OHSU | 0.032854 | Dead | NA | PTCL-NOS | PTCL-TBX21 |
| 219 | Validation | BCCA | 0.889802 | Dead | NA | PTCL-NOS | ATLL |
| | | (Vancouver BC) | | | | | |
| 220 | Validation | City of Hope | 0.280556 | Dead | NA | PTCL-NOS | ATLL |
| 221 | Validation | BCCA | 0.752778 | Dead | NA | PTCL-NOS | Intermediate |
| | | (Vancouver BC) | | | | | AITL/TBX21 |
| 222 | Validation | UNMC | 0.085 | Dead | NA | PTCL-NOS | PTCL-GATA3 |
| 223 | Validation | Wurzburg/ | NA | NA | NA | PTCL-NOS | PTCL-GATA3 |
| | | Stuttgart | | | | | |
| 224 | Validation | Wurzburg/ | NA | NA | NA | PTCL-NOS | PTCL-GATA3 |
| | | Stuttgart | | | | | |
| 225 | Validation | Wurzburg/ | NA | NA | NA | PTCL-NOS | PTCL-GATA3 |
| | | Stuttgart | | | | | |
| 226 | Validation | Wurzburg/ | NA | NA | NA | PTCL-NOS | PTCL-GATA3 |
| | | Stuttgart | | | | | |
| 227 | Validation | Wurzburg/ | NA | NA | NA | PTCL-NOS | PTCL-GATA3 |
| | | Stuttgart | | | | | |
| 228 | Validation | Wurzburg/ | NA | NA | NA | PTCL-NOS | PTCL-GATA3 |
| | | Stuttgart | | | | | |
| 229 | Validation | Cleveland | NA | NA | NA | PTCL-NOS | PTCL-GATA3 |
| | | Clinic | | | | | |
| 230 | Validation | Wurzburg/ | NA | NA | NA | PTCL-NOS | PTCL-GATA3 |
| | | Stuttgart | | | | | |
| 231 | Validation | Wurzburg/ | NA | NA | NA | PTCL-NOS | PTCL-GATA3 |
| | | Stuttgart | | | | | |
| 232 | Validation | Wurzburg/ | NA | NA | NA | PTCL-NOS | PTCL-GATA3 |
| | | Stuttgart | | | | | |
| 233 | Validation | Wurzburg/ | NA | NA | NA | PTCL-NOS | PTCL-GATA3 |
| | | Stuttgart | | | | | |
| 234 | Validation | Wurzburg/ | NA | NA | NA | PTCL-NOS | PTCL-TBX21 |
| | | Stuttgart | | | | | |
| 235 | Validation | Wurzburg/ | NA | NA | NA | PTCL-NOS | Intermediate |
| | | Stuttgart | | | | | AITL/TBX21 |
| 236 | Validation | Wurzburg/ | NA | NA | NA | PTCL-NOS | PTCL-TBX21 |
| | | Stuttgart | | | | | |
| 237 | Validation | Wurzburg/ | NA | NA | NA | PTCL-NOS | PTCL-TBX21 |
| | | Stuttgart | | | | | |
| 238 | Validation | Wurzburg/ | NA | NA | NA | PTCL-NOS | PTCL-TBX21 |
| | | Stuttgart | | | | | |
| 239 | Validation | Wurzburg/ | NA | NA | NA | PTCL-NOS | PTCL-TBX21 |
| | | Stuttgart | | | | | |
| 240 | Validation | Wurzburg/ | NA | NA | NA | PTCL-NOS | PTCL-TBX21 |
| | | Stuttgart | | | | | |
| 241 | Validation | Wurzburg/ | NA | NA | NA | PTCL-NOS | PTCL-TBX21 |
| | | Stuttgart | | | | | |
| 242 | Validation | BCCA | NA | NA | NA | PTCL-NOS | PTCL-GATA3 |
| | | (Vancouver BC) | | | | | |
| 243 | Validation | BCCA | NA | NA | NA | PTCL-NOS | PTCL-TBX21 |
| | | (Vancouver BC) | | | | | |
| 244 | Validation | BCCA | NA | NA | NA | PTCL-NOS | PTCL-TBX21 |
| | | (Vancouver BC) | | | | | |
| 245 | Validation | BCCA | NA | NA | NA | PTCL-NOS | PTCL-TBX21 |
| | | (Vancouver BC) | | | | | |
| 246 | Excluded | Wurzburg/ | NA | NA | NA | PTCL-TFH | Intermediate |
| | | Stuttgart | | | | | AITL/TBX21 |
| 247 | Excluded | BCCA | NA | NA | NA | PTCL-TFH | AITL |
| | | (Vancouver BC) | | | | | |
| 248 | Excluded | BCCA | NA | NA | NA | PTCL-TFH | Intermediate |
| | | (Vancouver BC) | | | | | AITL/TBX21 |
| 249 | Excluded | BCCA | NA | NA | NA | PTCL-TFH | PTCL-GATA3 |
| | | (Vancouver BC) | | | | | |
| 250 | Excluded | BCCA | NA | NA | NA | PTCL-TFH | Intermediate |
| | | (Vancouver BC) | | | | | AITL/TBX21 |
| 251 | Excluded | UNMC | NA | NA | NA | PTCL-TFH | PTCL-GATA3 |
| 252 | Excluded | Wurzburg/ | NA | NA | NA | PTCL-TFH | AITL |
| | | Stuttgart | | | | | |
| 253 | Excluded | OHSU | NA | NA | NA | PTCL-TFH | PTCL-TBX21 |
| 254 | Excluded | OHSU | NA | NA | NA | PTCL-TFH | PTCL-GATA3 |
| 255 | Excluded | Cleveland | NA | NA | NA | PTCL-TFH | Intermediate |
| | | Clinic | | | | | AITL/TBX21 |
| 256 | Excluded | UNMC | NA | NA | NA | PTCL-TFH | PTCL-GATA3 |
| 257 | Excluded | City of Hope | NA | NA | NA | PTCL-TFH | PTCL-TBX21 |
| 258 | Excluded | UNMC | NA | NA | NA | RH | PTCL-GATA3 |
| 259 | Excluded | UNMC | NA | NA | NA | RH | PTCL-GATA3 |
| 260 | Excluded | UNMC | NA | NA | NA | RH | PTCL-GATA3 |
| 261 | Excluded | UNMC | NA | NA | NA | RH | Intermediate |
| | | | | | | | AITL/GATA3 |
| 262 | Excluded | UNMC | NA | NA | NA | RH | PTCL-GATA3 |
| 263 | Excluded | UNMC | NA | NA | NA | RH | PTCL-GATA3 |
| 264 | Excluded | UNMC | NA | NA | NA | RH | PTCL-GATA3 |
| 265 | Excluded | UNMC | NA | NA | NA | RH | PTCL-TBX21 |
| 266 | Excluded | UNMC | NA | NA | NA | RH | PTCL-GATA3 |
| 267 | Excluded | UNMC | NA | NA | NA | RH | PTCL-GATA3 |

AITL, angioimmunoblastic T-cell lymphoma; ALCL, anaplastic large cell lymphoma; ATLL, adult T-cell lymphoma/leukemia; ENKTCL, extranodal NK/T-cell lymphoma; PTCL-NOS, peripheral T-cell lymphoma, not otherwise specified; PTCL-TFH, peripheral T-cell lymphoma with TFH phenotype; RH, reactive hyperplasia; NA: Not Available.

### Histopathology/Immunomorphological Features of the PTCL Cohorts

PTCL cases were centrally reviewed and diagnosed according to the current WHO classification⁶. The validation cohort was thoroughly re-evaluated by three hematopathologists (CA, DDW, WCC) with a comprehensive immunostaining panel and TCRg gene rearrangement analysis when needed. A consensus diagnosis was reached when there was unanimous agreement on the diagnosis.

### Rna Extraction and Digital Gene Expression for PTCL Subclassification

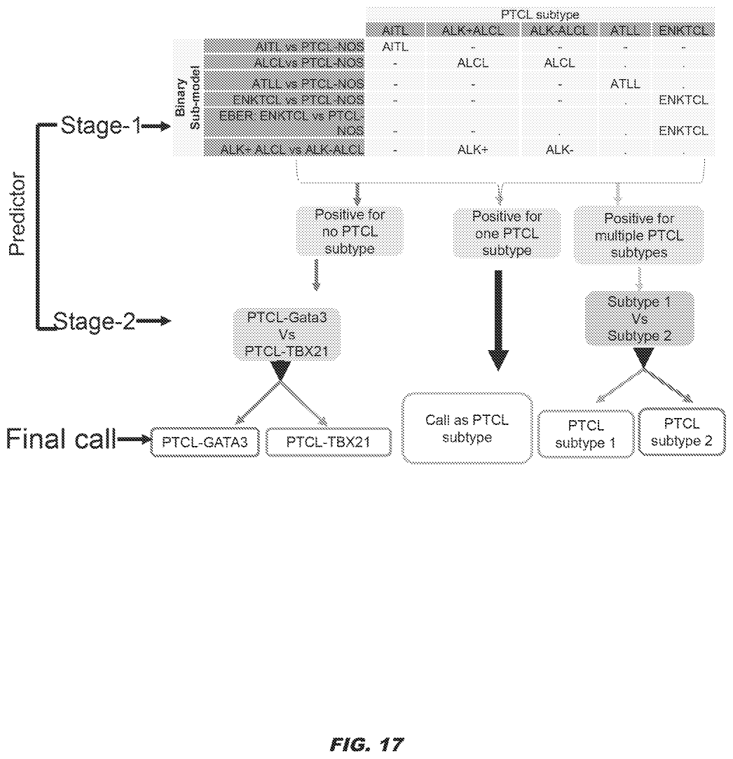

The details about RNA extraction protocols, quality control measures, nCounter® assay, data processing, cross-validation, and reproducibility assessment are in the supplemental section and in FIG. 12A. The data analysis and normalization were designed to process samples individually, rather than in batches, so that the protocol would be suitable for processing patient samples on an as needed basis. Class prediction was based on a series of binary comparisons that were combined to for a final classification call for each sample (FIG. 12A and FIG. 17). The detailed materials and methods are provided herein.

### Survival Analysis

The survival data were analyzed using the survival, survminer, and coin packages in R and detailed herein.

### Results

### Patient Characteristics in the “Training” and “Validation” Cohorts

The training cohort (n=105), which had previously generated GEP data in FF¹⁹⁻²¹ and matched FFPE tissues, was used to select the classifier genes for the nCounter® platform. The PTCL “validation” cohort (n=140) without fresh frozen transcriptomic data was rigorously diagnosed by 3-expert hematopathologists using current WHO diagnostic criteria⁶ (FIG. 12A and Tables 4 and 5). Of note, PTCL-NOS cases were evaluated with T_{FH} markers for exclusion of nodal PTCL-T_{FH} cases and subsequently subclassified into PTCL-GATA3 and PTCL-TBX21 using the recently published IHC algorithm²⁹.

The clinicopathological characteristics of the training and validation cohorts are summarized in Table 6. There was no significant difference in gender, age, and OS (FIG. 12C) between the validation and training cohorts. The median follow-up for survivors was 3.5 years (range 0.01-24 years) for patients with available survival data. ALK+ALCL cases showed a superior outcome than the other entities, consistent with published studies (FIG. 12D-E)^{2,21}.

### Development of Transcriptomic Signatures for FFPE Tissue

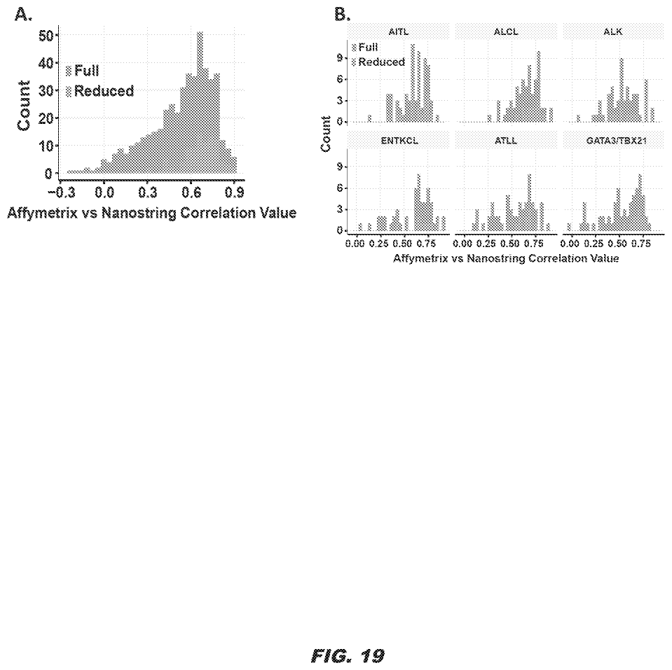

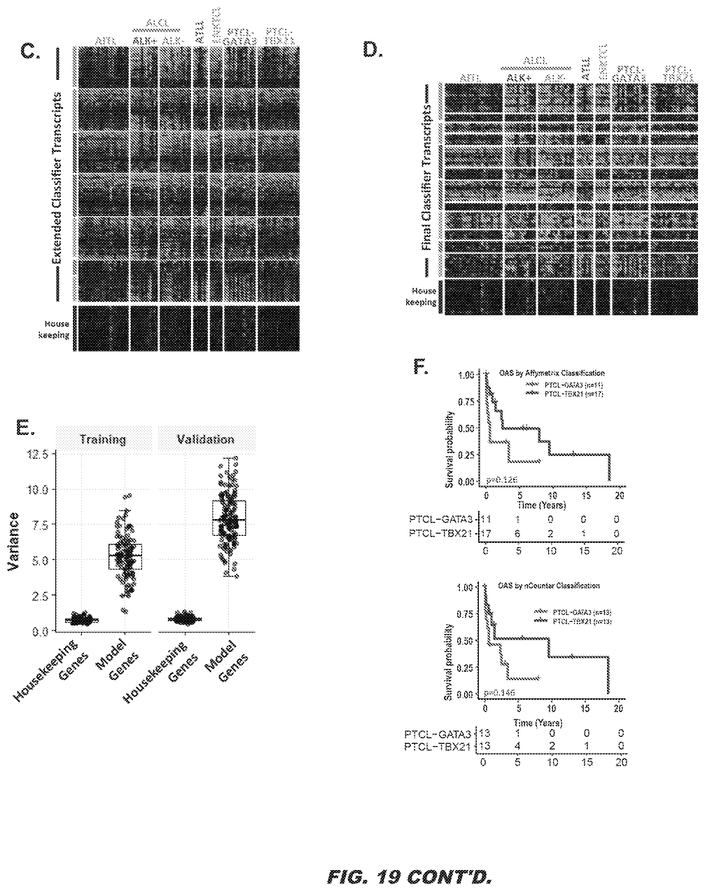

FFPE tissue blocks were selected based on the presence of adequate tumor tissue and RNA quality assessed as shown in supplemental-S1. Transcriptomic signatures assessed between two platforms (HG-U133plus2 (Affymetrix, Inc) versus the nCounter® platform revealed high correlation (correlation coefficient r>0.4) in the majority (˜60%) of the signature-specified genes (FIG. 19A-B). We performed recursive filtering analysis to exclude transcripts with correlation coefficients ≤0.4 using Pearson correlation (except 3 transcripts) to generate 11-20 diagnostic transcripts per PTCL subtype and 16 house-keeping genes (FIG. 19C-D). Using these well-performing transcripts on the nCounter® did not affect the classification accuracy sensitivity, specificity, either in fresh frozen RNA (HG-U133plus 2) or matched FFPE RNA (nCounter®, Inc) in the training cohort (n=105). The molecular subclassification using FFPE samples was highly comparable with the FF gold standard with an error rate of <5% across the various PTCL subtypes.

### Accuracy of PTCL Classification and Interlaboratory Reproducibility Using the Refined Signature

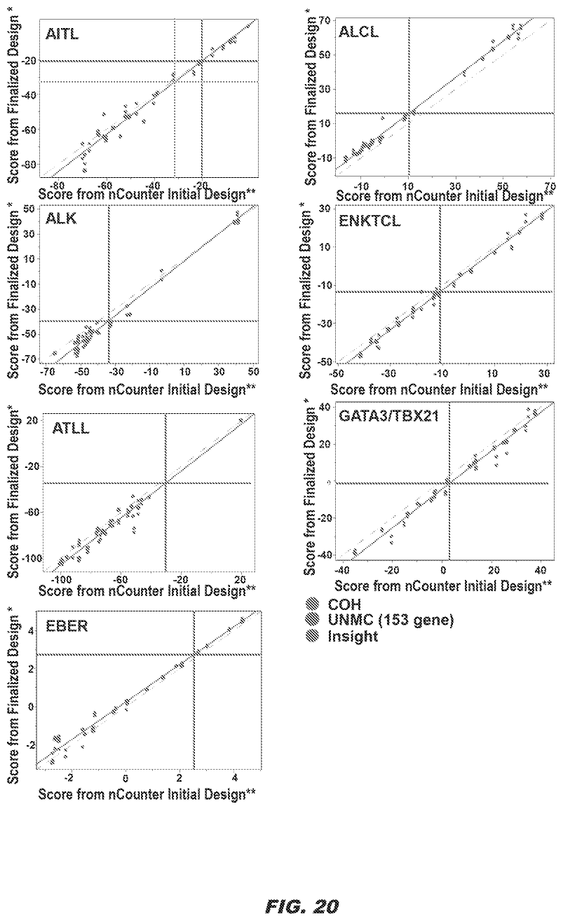

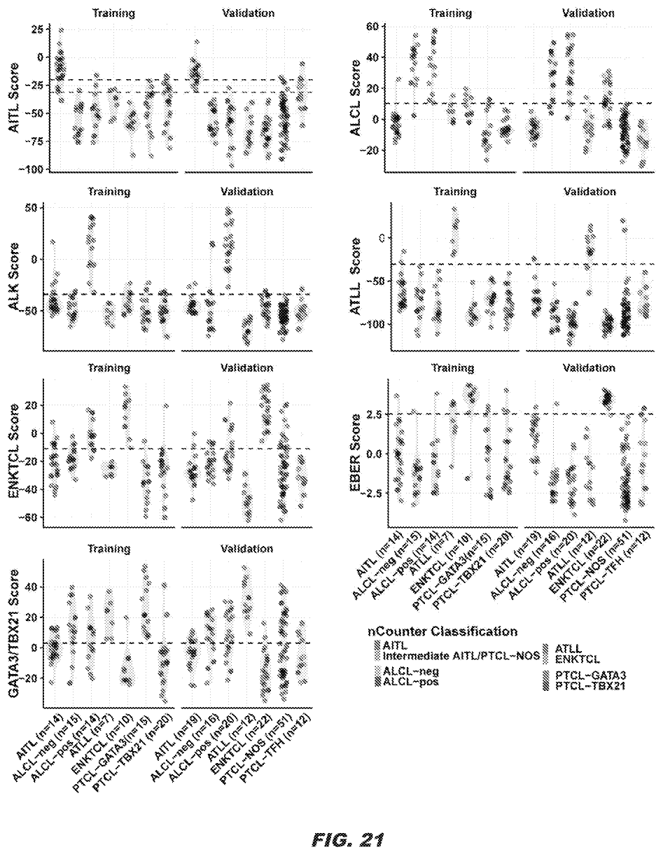

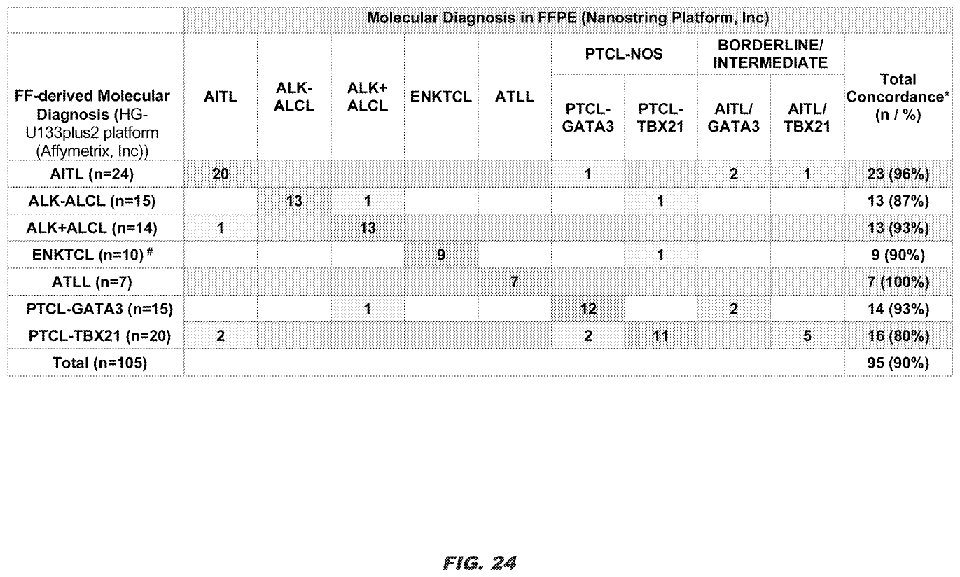

The reduced transcript signature retained the accuracy in classification in the HG-U133 plus2 platform data¹⁹⁻²¹ (FIG. 19C-D). Therefore, the reduced diagnostic transcripts were considered the “gold standard” for subsequent comparisons to the nCounter® assay in the training set (FIG. 12B, left panel). The classification of the FFPE training cohort was recapitulated on the nCounter® platform in 90% (95 of 105) of the cases (Tables 4, 5 and FIG. 24). We observed the prognostic difference between PTCL-GATA3 and PTCL-TBX21 (FIG. 19F), but the number of samples was too small to reach statistical significance. Since we transitioned from a larger panel to a smaller panel of diagnostic transcripts, seven cases from different PTCL subtypes were re-evaluated with the reduced transcript set versus the original larger panel on the nCounter® platform. They demonstrated concordant results, maintaining similar diagnostic accuracy (FIG. 20). When the assay was performed at two additional CLIA sites to assess the reproducibility, we observed highly concordant results and the same classification as at the original site for all 24 PTCL cases studied and interobserver (FIG. 20).

### Refined Diagnostic Algorithm Across Different PTCL Entities and Validation Cohort

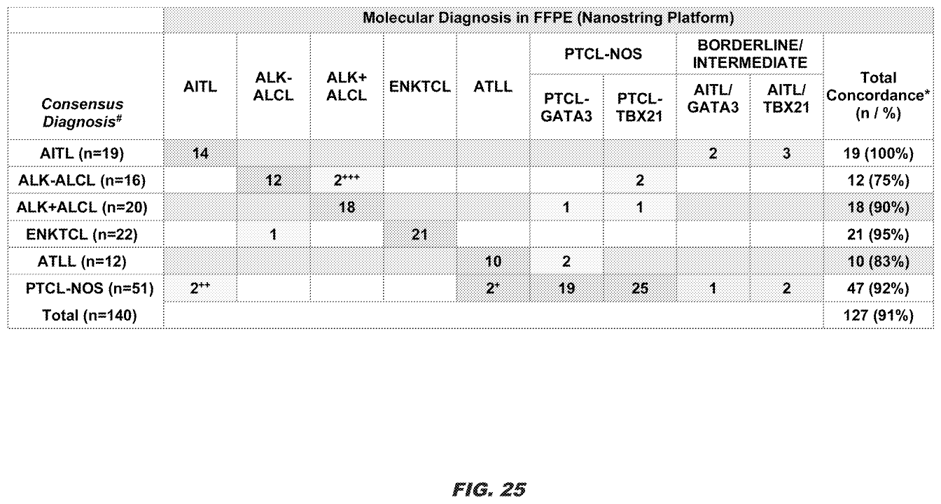

To assess diagnostic performance, the molecular classification obtained with the nCounter® platform was compared to the consensus pathologic diagnosis in an independent validation cohort (n=140). This cohort had a similar clinical outcome and distribution of PTCL subtypes as the training cohort (FIG. 12A,C). The classification obtained with the nCounter® platform was highly comparable with the diagnosis rendered by expert pathologists, with an overall concordance of 91% (127 of 140, 95% confidence interval (CI) 0.85-0.95) in the validation cases, and refined the classification of challenging PTCL cases as indicated below (Tables-4, 5 and FIG. 25; FIG. 12B, right panel; FIG. 21).

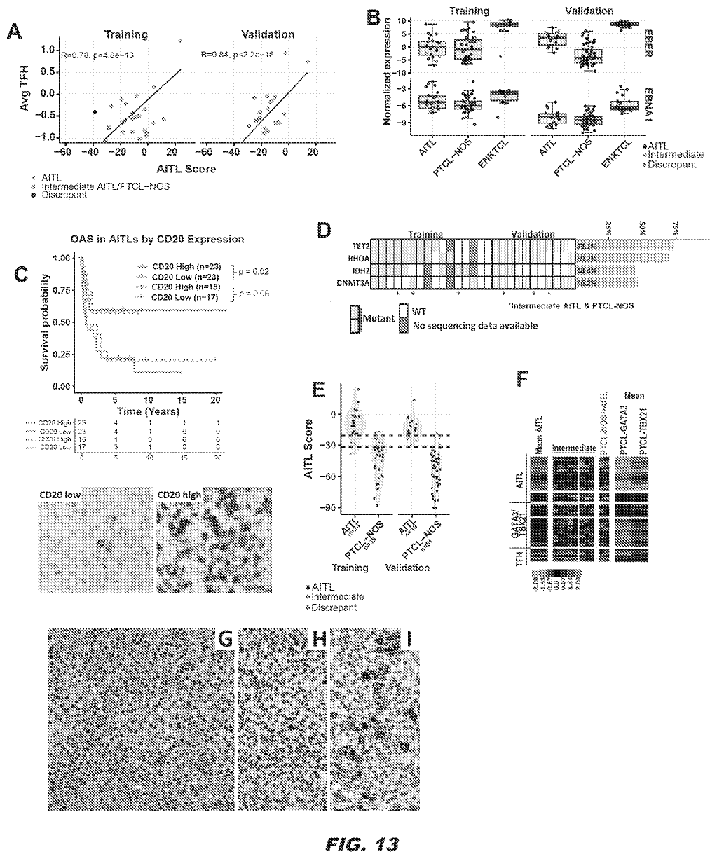

AITL: Average expression of the diagnostic signature significantly correlated with a pan T-FH gene expression signature (i.e., 6 transcripts defined in WHO as T_{FH} markers⁶) (FIG. 13A-B). The cases molecularly classified as AITL in the training and validation cohorts by nCounter® showed immunomorphological features commonly associated with AITL. Consistent with previous studies, high expression of CD20 was associated with a better OS^{20,21,30},which was validated using immunohistochemistry (FIG. 13C). An AITL mutation spectrum (i.e., TET2, DNMT3A, RHOA^{G17V}, and IDH2^{R172}) was seen in 89% of the cases with available sequencing data (FIG. 13D).

Using the nCounter® platform, AITL was classified with an 83% concordance in the training cohort (20/24) and 74% in the validation cohort (14/19), whereas the remaining cases (3 in training and 5 in validation) showed “borderline model score” between AITL and PTCL-NOS (Tables 4 and 5; FIG. 13E; FIG. 21). These cases missed the AITL molecular diagnosis based on the “threshold or cut point”. Upon re-review, these cases were confirmed to have classical AITL immunomorphological features, and mutation analysis supported the diagnosis, with a classical AITL mutation spectrum including TET2, RHOA^{G17V} and IDH2^{R172} as shown in FIG. 13D. These cases showed an AITL diagnostic signature expression at comparatively higher levels than other PTCLs, marginally lower than AITL, and a higher expression of the T_{FH} signature. (FIG. 13A,F). As expected, the three follicular T-cell lymphoma cases were all classified as AITL. Two PTCL-NOS cases were molecularly classified as AITL. These cases showed focal positivity of the T_{FH} markers BCL6 and ICOS (FIG. 13G-I), but criteria for nodal PTCL-T_{FH} were not met. While these two cases (FIG. 13F) clearly expressed AITL signature genes and T_{FH} mRNA signatures higher than most other PTCL-NOS cases, mutations in the genes commonly seen in AITL (TET2, IDH2^{R172}, RHOA^{G17V}, DNMT3A) were not present. These two cases may represent PTCL-T_{FH} that were not classified by the immunostains performed.

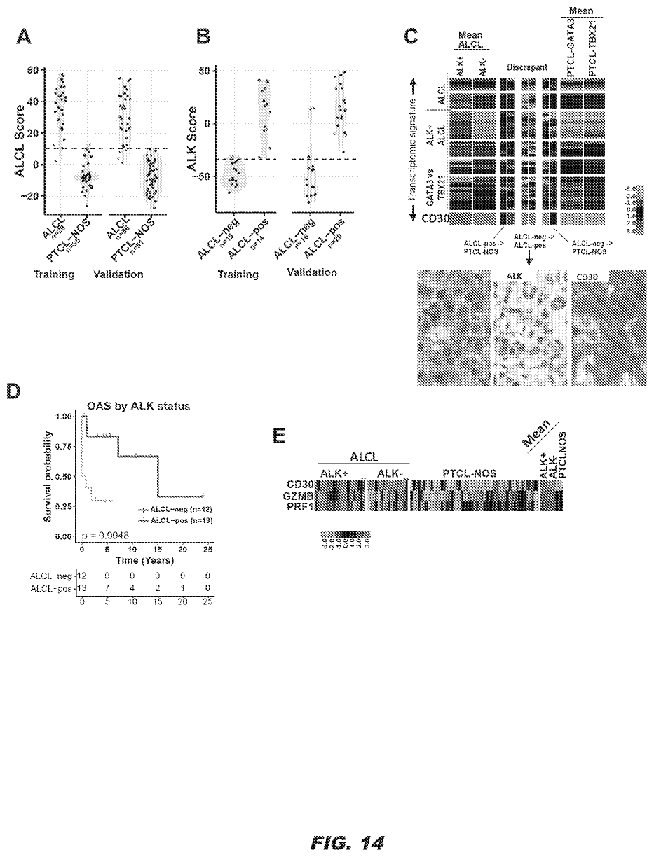

ALCL subtypes: Initial analysis of ALCL versus other PTCLs showed an 83% (30/36, 95% CI: 0.67-0.94) concordance in the validation cohort, which was comparable to the training cohort (26/29; 90%, 95% CI: 0.73-0.98) using the refined signature (FIG. 14A). The validation cohort (Tables 4 and 5; FIG. 14B) showed a 90% (18/20) concordance in ALK+ALCL and 75% (12/16) in ALK−ALCL. Interestingly, two ALK−ALCLs were classified as ALK+ALCL because of a high ALK+ALCL signature, but ALK mRNA expression was low (FIG. 14C), and IHC did not detect ALK protein expression. As expected, ALCL classified cases by ALK status showed that ALK-positive cases had a better OS (FIG. 14D). Remarkably, none of the PTCL-NOS cases, including those with strong CD30 positivity by IHC, were misclassified into ALCL. However, 4 ALCL (two ALK+ALCL and two ALK−ALCLs) showed a comparatively lower ALCL signature expression, although the two ALK+ALCLs had a high ALK signature (FIG. 14C). The two ALK-ALCL cases with low ALCL signature did not express CD30 mRNA, and the failure to classify these cases may be attributed to inadequate RNA quality or low tumor content (FIG. 14E). These findings suggest that the occasional cases with a low diagnostic signature should be diagnosed with caution.

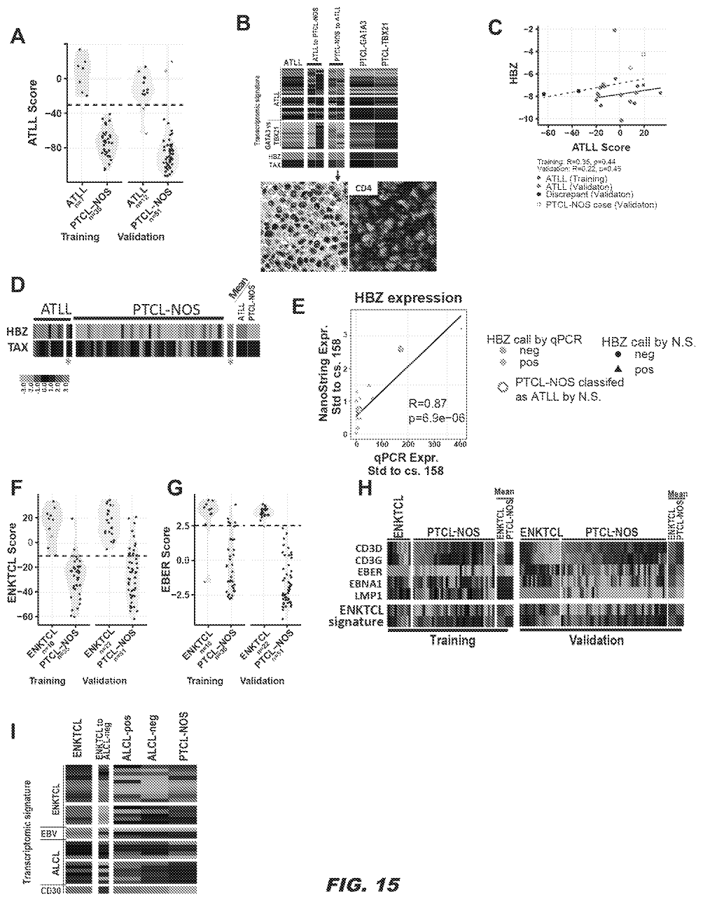

ATLL: The ATLL molecular signature detected 100% of ATLL cases in the training cohort (7/7, 95% CI: 0.59-1) and 83% of the validation cohort cases (10/12, 95% CI: 0.52-0.98) (FIG. 15A). The 2 cases showing disagreement had marginal expression of the ATLL diagnostic signature, but both were confirmed to be positive for HTLV1 mRNA expression (i.e., HBZ by qPCR), though in one, the expression of HBZ measured by the nCounter® was lower than other ATLLs (FIG. 15B-C). Of the two HTLV1 transcripts (HZB and TAX1), HZB was consistently expressed at higher levels in ATLL cases compared to TAX and showed a positive correlation with the ATLL signature (FIG. 15D-E), and a significant correlation with HBZ expression measured by qRT-PCR (FIG. 15F). Interestingly, two PTCL-NOS cases from the validation cohort were molecularly classified as ATLL (FIG. 15A). Re-evaluation of these cases using HZB-specific qPCR confirmed the expression of HBZ (FIG. 15E), and subsequent review of the clinical chart indicated serologic positivity for HTLV1 and a clinical presentation compatible with ATLL, unknown at the time of the initial diagnosis. Morphologically, these cases consisted of CD4-positive monomorphic T-cell lymphomas which, in the absence of an appropriate clinical history, blood examination, and serologic testing, were misdiagnosed as PTCL-NOS⁷ (FIG. 15B). Therefore, these two cases were reclassified as ATLL.

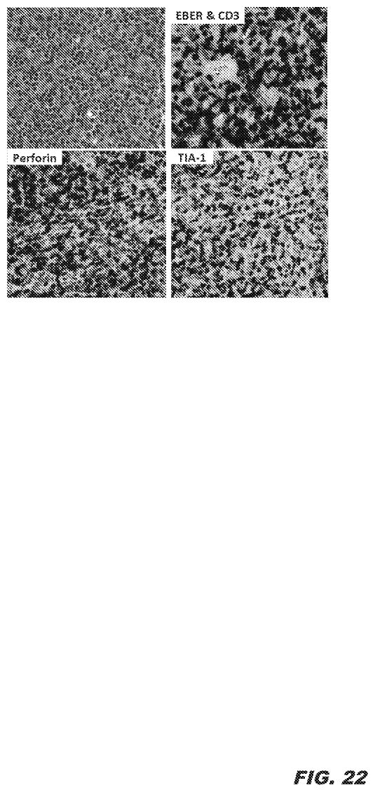

ENKTCL: The ENKTCL molecular classifier was able to identify 90% (9/10) of ENKTCL cases in the training cohort and 95% (21/22) in the validation cohort (Tables 4 and 5, FIG. 15F-G). A subset of cases had elevated expression of CD3γ and CD3δ relative to other cases (FIG. 15H) and may be derived from the T-lineage. Two cases were diagnosed according to current WHO classification as “primary EBV-positive nodal T/NK-cell lymphoma”. These cases resemble ENKTCL but with a primarily nodal presentation, and both were classified as ENKTCL by the molecular assay (FIG. 22). One molecularly classified ENTKCL case uniquely showed both ALCL and ENKTCL signatures but strong expression of EBV transcripts (FIG. 14I).

### Refinement and Validation of Two Novel PTCL-NOS Subtypes (PTCL-GATA3 and PTCL-TBX21)

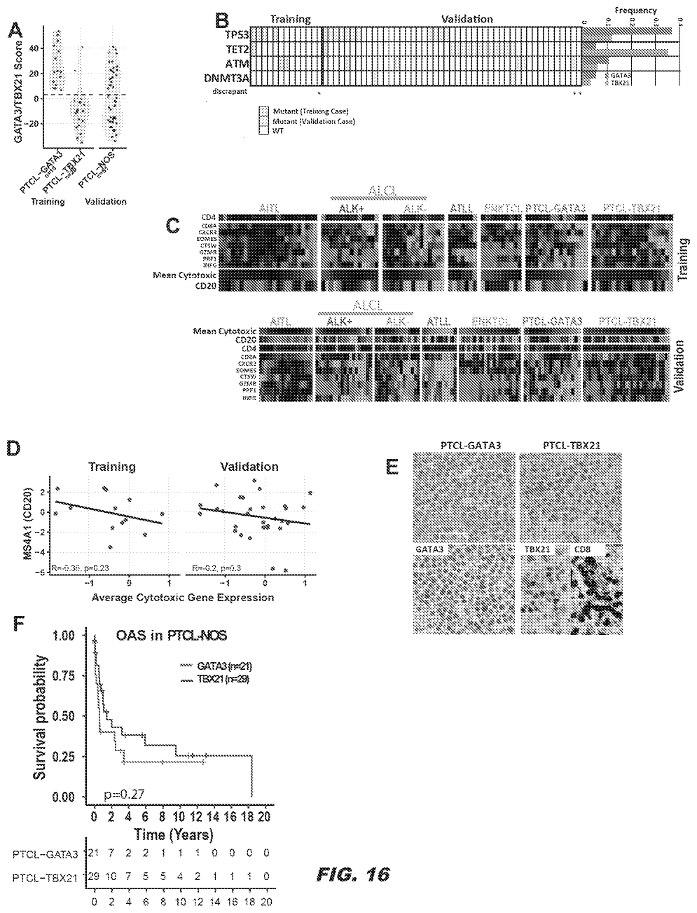

Of the PTCL-NOS cases in the training cohort, we separated two novel molecular subgroups with an 87% concordance (i.e., PTCL-GATA3 or PTCL-TBX21) with a reduced number of transcripts (n=19). Of the remaining PTCL-NOS group in the validation, 40% (21/52) were classified as PTCL-GATA3 subtype and 52% (27/52) as the PTCL-TBX21 subtype using the nCounter® platform (Tables 4 and 5; FIG. 16A). This classification showed good concordance with our IHC algorithm classification (overall concordance: 80%). To further validate our transcriptomic signature classification, we compared sequencing data in these two groups. We observed that genetic alterations like TET2 mutation were frequent in the PTCL-TBX21 subtype, whereas TP53 mutations were enriched in the PTCL-GATA3 subgroup, concordant with our prior findings³¹ (FIG. 16B).

Consistent with our prior observations^{21,29}, cases with the expression of cytotoxic transcripts were significantly enriched in the PTCL-TBX21 subtype (FIG. 16C) and validated by a more frequent cytotoxic immunophenotype in the PTCL-TBX21 subtype than in the PTCL-GATA3 subtype (52% vs. 16%, p=0.013, FIG. 16C). An inverse correlation was noted between the average cytotoxic CD8+ T-cell signature and the pan B-cell CD20 transcripts (FIG. 16D). Consistent with our prior observation²⁹, PTCL-TBX21 cases frequently had an enriched inflammatory background irrespective of the cytotoxic phenotype (FIG. 16E). Since the clinical outcome of the PTCL-NOS was available only in a limited number of cases, but the combined cohort trended toward inferior OS associated with cases classified as PTCL-GATA3 vs. PTCL-TBX21 (median OS: 0.57 vs. 1.4 years; FIG. 16F).

Evaluation of the PTCL-T_{F}H Cases, Excluded from the Validation Cohort

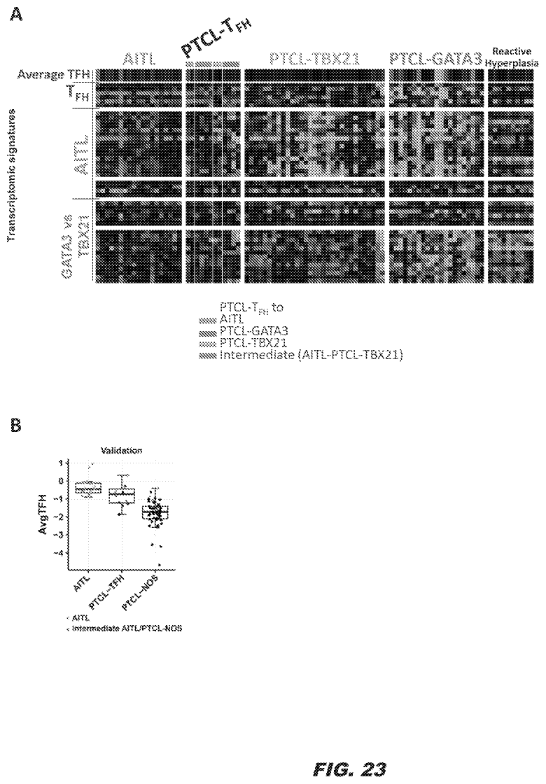

PTCL-T_{FH} (n=12) cases were analyzed using the nCounter® platform, and only 2 (17%) showed a significant association with the AITL molecular signature, whereas 4 cases had borderline scores between AITL and PTCL-NOS, and 6 cases showed a clear association with the PTCL-NOS, resembling PTCL-TBX21 cases (n=3) or PTCL-GATA3 (n=3). When we specifically examined the T_{FH} signature, the PTCL-T_{FH} cases with high AITL signatures also showed higher T_{FH} signatures than the rest (FIG. 23A-B). We also included 10 cases of reactive hyperplasia, and none of them showed expression of the diagnostic signatures for subtypes of PTCL included in the assay.

### Discussion

The diagnosis of PTCL is one of the most challenging among lymphomas and more often results in an inconclusive, inconsistent, or incorrect diagnosis^{19-21,32,33}. Recently, novel therapeutic approaches have shown striking benefits in subgroups of PTCL, including Brentuximab-Vedotin (BV) on CD30+ PTCL, particularly ALCL³⁴, Crizotinib in ALK+ALCL^{35,36}; mogamulizumab in ATLL³⁷, HDACi and demethylating agents in AITL or T_{FH}-PTCL³⁸, and possibly Enasidenib in IDH2 mutant AITL³⁹. Thus, accurate diagnosis may be important for patient treatment and in clinical trials of new drugs⁴⁰⁻⁴². We have performed extensive GEP studies on PTCL and constructed RNA-based molecular diagnostic signatures and predictors of survival and have delineated critical oncogenic mechanisms¹⁹⁻²¹. Some of these findings have been included in the revised 2016 WHO classification⁶. To translate this molecular information to a platform suitable for clinical application²⁵, we performed a systematic analysis to identify RNAs from FFPE tissues in PTCL, using the nCounter® platform that correlated well with GEP data from fresh frozen tissues, as this digital quantitation technology is more tolerant of degraded RNA typical of FFPE materials. In addition, we developed a diagnostic transcriptomic signature with a minimum number of transcripts that performed comparably in the training set with the previous GEP derived diagnosis¹³⁻¹⁵. In the pre-analytical assessment, RNA yield and quality (i.e., DV₂₀₀>50%) of older specimens were improved using the RNAstorm™ kit. However, in recently acquired FFPE tissues, other isolation methods may also provide good quality RNA. Additionally, this assay could be performed using limited RNA quantities (minimum required 200 ng), usually obtained from a few unstained slides depending on the size of the tissue. However, we were able to have an adequate classification even using RNA extracted from core needle biopsies, which represented ˜10% of the study samples. The inter-lab comparison of variability and reproducibility across three CLIA certified labs also correlated very well.

As we do not have GEP data on cases for validation, we only included cases with a firm pathology diagnosis after extensive IHC studies and stringent review. The findings were very similar to those of the training set (FIG. 12), maintaining high sensitivity, specificity, and accuracy. In some cases, the signature score fell just outside the cut-off points, and these were considered as “borderline” cases. It is reassuring that a few cases in which the molecular assays made the correct diagnosis on a retrospective analysis.−. However, frank discrepancies were also observed. This could be partly due to technical reasons such as tumor content and heterogeneity of the tumor, but some may be related to biology that was not known yet, such as a strong ALK signature in some ALK-ALCL cases that may represent the recently reported ALK-positive-like ALCL⁴³. Similarly, two PTCL-NOS showed significant association with AITL and T_{FH} transcriptomics signature, which upon review, showed focal expression of T_{FH} markers (BCL6, ICOS). The strong T_{FH} expression signature may indicate that these cases are more similar to PTCL-T_{FH} but did not meet the current criteria of strong expression of at least two T_{FH} markers. In these cases, the molecular assay revealed the complex and overlapping biology between PTCL-NOS and AITL and their poorly defined borders. We also found that the addition of EBV and HTLV1 transcripts enhanced the diagnostic performance of the molecular assay in ENTKCL and ATLL, respectively. One important contribution of this approach was the robust definition of the PTCL-GATA3 and PTCL-TBX21 cases, which have different biology and prognosis as supported by recent genetic findings³¹. While it is possible to simulate the GEP classification using an IHC panel, the stains can be challenging to optimize and interpret, which may lead to substantial variability among institutions. The assay reported here was highly reproducible among laboratories and, thus, presents a major advantage.

The GEP study that initially defined the diagnostic signatures was performed before the definition of the provisional entity of PTCL-T_{FH} was not formally included in this study. However, the 12 PTCL-T_{FH} cases explored with the nCounter® assay showed higher mean AITL and T_{FH} signatures, and individual signatures overlapped with the molecular signatures for AITL or PTCL-subtypes, similar to the study by Dobay et al.⁴⁴. These exploratory studies indicated that PTCL-T_{FH} is unlikely to be a single entity as currently defined and needs further evaluation to determine how best it should be characterized. Similarly, our previous GEP studies^{20,21} indicated a cytotoxic PTCL variant within the PTCL-TBX21 subgroup. Consistent with that observation, we found 25% of the cases in PTCL-TBX21 to have a cytotoxic signature and validated an association with CD8+ phenotype by IHC. Due to the small number of cases studied, further investigations are necessary to develop a robust signature for this group of cases.

In summary, we described an approach to translate the PTCL diagnostic signatures into a clinically applicable assay, which we envision to be a useful tool for general and academic pathologists in the diagnostically challenging field of PTCL. Additionally, we believe it can facilitate a better definition of cases for research studies and ensure more uniform cohorts for clinical trials. PTCL classification is an evolving area, and as our understanding of the underlying biology and available technology improves, modifications will be instituted to make the classification clinically relevant.

### References Cited in this Example

1. Bellei M, Chiattone C S, Luminari S, et al. T-cell lymphomas in South America and europe. Rev Bras Hematol Hemoter. 2012; 34(1):42-47.
2. Vose J, Armitage J, Weisenburger D, International TCLP. International peripheral T-cell and natural killer/T-cell lymphoma study: pathology findings and clinical outcomes. J Clin Oncol. 2008; 26(25):4124-4130.
3. Briski R, Feldman A L, Bailey N G, et al. The role of front-line anthracycline-containing chemotherapy regimens in peripheral T-cell lymphomas. Blood Cancer J. 2014; 4:e214.
4. Rudiger T, Weisenburger D D, Anderson J R, et al. Peripheral T-cell lymphoma (excluding anaplastic large-cell lymphoma): results from the Non-Hodgkin's Lymphoma Classification Project. Ann Oncol. 2002; 13(1):140-149.
5. Fox C P, Civallero M, Ko Y H, et al. Survival outcomes of patients with extranodal natural-killer T-cell lymphoma: a prospective cohort study from the international T-cell Project. Lancet Haematol. 2020;7(4):e284-e294.
6. Swerdlow S H, Campo E, Harris N L, et al. WHO Classification of Tumours of Haematopoietic and Lymphoid Tissues, Fourth Edition. Revised. 2017;2.
7. Weisenburger D D, Savage K J, Harris N L, et al. Peripheral T-cell lymphoma, not otherwise specified: a report of 340 cases from the International Peripheral T-cell Lymphoma Project. Blood. 2011; 117(12):3402-3408.
8. Morris S W, Kirstein M N, Valentine M B, et al. Fusion of a kinase gene, ALK, to a nucleolar protein gene, NPM, in non-Hodgkin's lymphoma. Science. 1994; 263(5151):1281-1284.
9. Tsukasaki K, Tsushima H, Yamamura M, et al. Integration patterns of HTLV-I provirus in relation to the clinical course of ATL: frequent clonal change at crisis from indolent disease. Blood. 1997; 89(3):948-956.
10. Escalon M P, Liu N S, Yang Y, et al. Prognostic factors and treatment of patients with T-cell non-Hodgkin lymphoma: the M. D. Anderson Cancer Center experience. Cancer. 2005; 103(10):2091-2098.
11. Horwitz S, O'Connor O A, Pro B, et al. Brentuximab vedotin with chemotherapy for CD30-positive peripheral T-cell lymphoma (ECHELON-2): a global, double-blind, randomised, phase 3 trial. Lancet. 2019; 393(10168):229-240.
12. Pro B, Advani R, Brice P, et al. Five-year results of brentuximab vedotin in patients with relapsed or refractory systemic anaplastic large cell lymphoma. Blood. 2017; 130(25):2709-2717.
13. Alizadeh A A, Eisen M B, Davis R E, et al. Distinct types of diffuse large B-cell lymphoma identified by gene expression profiling. Nature. 2000; 403(6769):503-511.
14. Rosenwald A, Wright G, Chan W C, et al. The use of molecular profiling to predict survival after chemotherapy for diffuse large-B-cell lymphoma. N Engl J Med 2002; 346(25):1937-1947.
15. Dave S S, Fu K, Wright G W, et al. Molecular diagnosis of Burkitt's lymphoma. N Engl J Med 2006; 354(23):2431-2442.
16. Monti S, Savage K J, Kutok J L, et al. Molecular profiling of diffuse large B-cell lymphoma identifies robust subtypes including one characterized by host inflammatory response. Blood 2005; 105(5):1851-1861.
17. Rosenwald A, Wright G, Wiestner A, et al. The proliferation gene expression signature is a quantitative integrator of oncogenic events that predicts survival in mantle cell lymphoma. Cancer Cell. 2003b; 3(2):185-197.
18. Shipp M A, Ross K N, Tamayo P, et al. Diffuse large B-cell lymphoma outcome prediction by gene-expression profiling and supervised machine learning. Nat Med. 2002; 8(1):68-74.
19. Iqbal J, Weisenburger D D, Chowdhury A, et al. Natural killer cell lymphoma shares strikingly similar molecular features with a group of non-hepatosplenic gammadelta T-cell lymphoma and is highly sensitive to a novel aurora kinase A inhibitor in vitro. Leukemia. 2011; 25(2):348-358.
20. Iqbal J, Weisenburger D D, Greiner T C, et al. Molecular signatures to improve diagnosis in peripheral T-cell lymphoma and prognostication in angioimmunoblastic T-cell lymphoma. Blood. 2010; 115(5):1026-1036.
21. Iqbal J, Wright G, Wang C, et al. Gene expression signatures delineate biological and prognostic subgroups in peripheral T-cell lymphoma. Blood. 2014; 123(19):2915-2923.
22. Lone W, Alkhiniji A, Manikkam Umakanthan J, Iqbal J. Molecular Insights Into Pathogenesis of Peripheral T Cell Lymphoma: a Review. Curr Hematol Malig Rep. 2018; 13(4):318-328.
23. Iqbal J, Amador C, McKeithan T W, Chan W C. Molecular and Genomic Landscape of Peripheral T-Cell Lymphoma. Cancer Treat Res. 2019; 176:31-68.
24. Srinivasan M, Sedmak D, Jewell S. Effect of fixatives and tissue processing on the content and integrity of nucleic acids. Am J Pathol. 2002; 161(6):1961-1971.
25. Crescenzo R, Abate F, Lasorsa E, et al. Convergent mutations and kinase fusions lead to oncogenic STAT3 activation in anaplastic large cell lymphoma. Cancer cell. 2015; 27(4):516-532.
26. Scott D W, Wright G W, Williams P M, et al. Determining cell-of-origin subtypes of diffuse large B-cell lymphoma using gene expression in formalin-fixed paraffin-embedded tissue. Blood. 2014; 123(8):1214-1217.
27. Phang K C, Akhter A, Tizen N M S, et al. Comparison of protein-based cell-of-origin classification to the Lymph2Cx RNA assay in a cohort of diffuse large B-cell lymphomas in Malaysia. J Clin Pathol. 2018; 71(3):215-220.
28. Rimsza L M, Wright G, Schwartz M, et al. Accurate classification of diffuse large B-cell lymphoma into germinal center and activated B-cell subtypes using a nuclease protection assay on formalin-fixed, paraffin-embedded tissues. Clin Cancer Res. 2011; 17(11):3727-3732.
29. Amador C, Greiner T C, Heavican T B, et al. Reproducing the molecular subclassification of peripheral T-cell lymphoma-NOS by immunohistochemistry. Blood. 2019; 134(24):2159-2170.
30. Advani R H, Ansell S M, Lechowicz M J, et al. A phase II study of cyclophosphamide, etoposide, vincristine and prednisone (CEOP) Alternating with Pralatrexate (P) as front line therapy for patients with peripheral T-cell lymphoma (PTCL): final results from the T-cell consortium trial. Br J Haematol. 2016; 172(4):535-544.
31. Heavican T B, Bouska A, Yu J, et al. Genetic drivers of oncogenic pathways in molecular subgroups of peripheral T-cell lymphoma. Blood. 2019; 133(15):1664-1676.
32. Maura F, Agnelli L, Leongamornlert D, et al. Integration of transcriptional and mutational data simplifies the stratification of peripheral T-cell lymphoma. Am J Hematol. 2019.
33. Piccaluga P P, Fuligni F, De Leo A, et al. Molecular profiling improves classification and prognostication of nodal peripheral T-cell lymphomas: results of a phase III diagnostic accuracy study. J Clin Oncol. 2013; 31(24):3019-3025.
34. Van Der Weyden C, Dickinson M, Whisstock J, Prince H M. Brentuximab vedotin in T-cell lymphoma. Expert Rev Hematol. 2019; 12(1):5-19.
35. Andraos E, Dignac J, Meggetto F. NPM-ALK: A Driver of Lymphoma Pathogenesis and a Therapeutic Target. Cancers (Basel). 2021;13(1).
36. Gambacorti-Passerini C, Messa C, Pogliani E M. Crizotinib in anaplastic large-cell lymphoma. N Engl J Med. 2011; 364(8):775-776.
37. Moskowitz A J, Lunning M A, Horwitz S M. How I treat the peripheral T-cell lymphomas. Blood. 2014; 123(17):2636-2644.
38. Lopez A T, Bates S, Geskin L. Current Status of HDAC Inhibitors in Cutaneous T-cell Lymphoma. Am J Clin Dermatol. 2018; 19(6):805-819.
39. https://clinicaltrials.gov/ct2/show/NCT02273739.
40. Ishida T, Joh T, Uike N, et al. Defucosylated anti-CCR4 monoclonal antibody (KW-0761) for relapsed adult T-cell leukemia-lymphoma: a multicenter phase II study. J Clin Oncol. 2012; 30(8):837-842.
41. O'Connor O A, Bhagat G, Ganapathi K, et al. Changing the paradigms of treatment in peripheral T-cell lymphoma: from biology to clinical practice. Clin Cancer Res. 2014; 20(20):5240-5254.
42. Iqbal J, Wilcox R, Naushad H, et al. Genomic signatures in T-cell lymphoma: How can these improve precision in diagnosis and inform prognosis? Blood Rev. 2016; 30(2):89-100.
43. Yeung K H R A, Russell V, Choi W, et al. ALK-Negative Anaplastic Large Cell Lymphomas Encompass Distinct Subgroups Including an ALK-Positive-like Subgroup with Favorable Prognosis. Blood. 2021;138 2403.
44. Dobay M P, Lemonnier F, Missiaglia E, et al. Integrative clinicopathological and molecular analyses of angioimmunoblastic T-cell lymphoma and other nodal lymphomas of follicular helper T-cell origin. Haematologica. 2017;102(4):e148-e151.

### Materials and Methods

### Patient Information

We included 249 diagnostic PTCL cases from multiple institutions, which after exclusion of 4 cases with poor RNA quality, were then divided into a training cohort of 105 cases and a validation cohort of 140 cases (Tables 4 and 5/FIG. 12A). The basic clinical and pathological characteristics of the two cohorts are shown in Table 6. The training cohort included a “bridging set” of 91 cases that had been previously evaluated in prior GEP studies¹⁻³ using RNA from FF tissue on an HG-U133 Plus 2.0 Array13-15 (Affymetrix, Santa Clara, CA; accession number GSE19069) and which had available corresponding FFPE samples. In addition, 9 new PTCLs with available HG-U133 Plus 2.0 data that were not included in the earlier GEP studies¹⁻³ and 5 well-characterized, classical ENKTCL cases with a confirmed diagnosis were also included in the training set. The training cohort consisted of 24 AITL, 14 ALK+ALCL, 15 ALK−ALCL, 7 ATLL, 10 ENKTCL, and 35 PTCL-NOS (15 PTCL-GATA3 and 20 PTCL-TBX21). These were used to identify which RNA transcripts previously identified as diagnostic on FF tissue also performed well in FFPE on the nCounter® platform (NanoString Inc, Seattle).

A separate validation cohort consisted of an independent series of 140 PTCL cases, not previously analyzed with GEP but with adequate FFPE tissues available. This set included 19 AITL, 20 ALK+ALCL, 16 ALK−ALCL, 12 ATLL, 22 ENKTCL, and 51 PTCL-NOS. Though we initially assembled 152 PTCL cases for validation, 12 were excluded from this cohort since they were pathologically reclassified as PTCL with a T-follicular helper (T_{FH}) phenotype. These 12 PTCL-T_{FH} cases and 10 additional reactive lymphadenopathies were analyzed with the NanoString-based assay for comparison. The research protocol was approved by the UNMC and COH-MC Institutional Review Board.

### Morphological and Immunophenotypical Characteristics of the PTCL Cohorts

All cases were centrally reviewed and diagnosed according to the current WHO classification⁴. The cases in the validation cohort consisted of a new cohort of cases without FF GEP data that underwent a rigorous pathological review in a consensus conference by three hematopathologists (CA, DW, WCC) with expertise in T-cell lymphoma diagnosis. A consensus diagnosis was reached when there was unanimous agreement on the diagnosis. The pathological evaluation included part or entire panel of B and T-cell immunostains including CD3, CD20, CD30, T_{FH} markers (PD1, ICOS, CD10, CXCL13, and BCL6), CD4, CD8, cytotoxic markers (TIA1, granzyme B, and perforin), TCRαβ, TCRγδ, and EBER in-situ hybridization as needed. Cases of PTCL-NOS with strong expression of two or more T_{FH} markers were reclassified as nodal PTCL with T_{FH} phenotype. PTCL-NOS cases were subclassified into PTCL-GATA3 and PTCL-TBX21 using the recently published IHC algorithm using antibodies against GATA3, TBX21, CCR4, and CXCR3 by one hematopathologist (CA)⁵. Of the 43 AITL cases, 40 had typical AITL morphology, but three were T-FL grouped with AITL for transcriptomic analysis. ATLL cases in the validation series must have a positive signal (CT-value) with HZB viral transcript using qRT-PCR (Taq man probe) based on FFPE-extracted RNA, according to a previously established test for the presence of HTLV-1 virus in ATLL^{4,6,7}. ENKTCL cases in the validation cohort were confirmed using either EBER in situ hybridization or PCR for the existence of LMP1/EBNA1 in the genomic DNA. Of the 32 ENKTCL cases, 30 had nasal involvement and were typical ENKTCL, and two had primarily nodal involvement but otherwise had similar phenotypic features as the nasal ENKTCL cases. Detailed information on nasal involvement in these 2 cases was not available. The two cases may represent primary nodal EBV-positive T/NK-cell lymphoma in the current WHO classification, but were grouped with ENK/TCL for transcriptomic analysis.

RNA Extraction from FFPE Tissues and Digital Gene Expression Using nCounter® System

FFPE scrolls of 10-20 m thickness were cut from blocks to a surface area of 1 cm2 and stored at −20° C. until extraction. For a few cases where FFPE scrolls were not available, tissue was scraped from unstained slides. The de-paraffinization and extraction of total-RNA was performed using two commercial kits (a) Qiagen AllPrep DNA/RNA FFPE Kit (Qiagen GmbH, Germany) or (b) RNAStorm™ RNA isolation kit (Cell Data Sciences, CD501)⁸ according to the manufacturer's instructions. The RNA quality was assessed using Agilent Tape-Station and estimated using RNA integrity analysis (RIN) value⁹ and by the RNA fragment size >200 bases DV₂₀₀)¹⁰. The RNA was quantified using Qubit, and 200 ng of total RNA (DV200:>35%) was used for hybridization, and for more degraded samples, additional RNA was used, following NanoString, Inc recommendations (https://www.nanostring.com/wp-content/uploads/2021/07/MAN-10050-05-Preparing-48 RNA-from-FFPE-Samples.pdf). The total RNA was hybridized to the custom codeset (see below) at 65° C. overnight (16 to 18 hours) on the nCounter® Prep Station and GEP data was acquired on the nCounter® digital analyzer at the “high 50 resolution” setting. Standard QC (as offered by nSolver™ analysis software, NanoString Technologies) was employed, with flagging of any sample with a total of the positive spike-in controls outside of 0.3 to 3 times the geometric mean of the total positive spike-in for that cartridge. Signal count values were normalized by dividing the counts for each gene for a given sample by the geometric mean of the counts for the housekeeping genes in that sample. The normalized counts were then log 2 transformed to obtain gene signal values utilized in all subsequent analyses. Samples for which the geometric mean of the housekeeping genes was 10 or less were excluded from the study.

Code Set Design for the nCounter® PTCL Subtyping Assay

The training cohort of PTCL cases (n=105), with GEP derived from fresh frozen RNA, was used to select the classifier genes for the nCounter® platform using corresponding FFPE tissues. We reanalyzed the earlier FF GEP data (HG-U133 Plus 2.0 Array, Affymetrix, Inc) to generate a diagnostic transcript signature, which included 287 classifier transcripts specific for PTCL subtypes, 50 housekeeping genes with consistent expression in PTCL, an extra set of genes associated with T-cell differentiation. The genes were selected either to be characteristically differentially expressed (either positively or negatively) for: (1) one of the PTCL subtypes (i.e., AITL, ALCL, ENKTCL, ATLL), (2) be differentially expressed between ALK+ and ALK−ALCL, or (3) to distinguish between the PTCL-TBX21 and PTCL-GATA3 subgroups of PTCL-NOS. The 50 housekeeping genes were selected among those with the lowest variance across the PTCL in the FF GEP data. Thus, these classifier transcripts were designed to be the most informative and robust elements for classification, and nCounter® CodeSets were designed with the NanoString bioinformatics team using the standard nCounter® chemistry for CodeSet design. The barcode corresponding to each captured transcript was imaged and counted post hybridization, thus providing digital quantitation of each captured RNA (details about custom CodeSet for genes can be viewed at https://www.nanostring.com/products/custom-solutions/custom-CodeSets).

To minimize the cost associated with recurring CodeSet design and manufacturing (nCounter®, NanoString Inc), we used the samples from the bridging cohort to curate the initial set of diagnostic markers to define a set of 93 genes and five subtype-specific viral transcripts from EBV and HTLV-1 which showed the best performance based on the NanoString platform. The following sets of criteria were used to make this evaluation: (a) correlation between the NanoString log 2 signal values and their corresponding HG-U133 Plus 2.0 signal intensity (SI) values; (b) T-statistic between the normalized log-transformed NanoString SI values for those samples in the PTCL subtype vs. other PTCL subtypes; (c) T-statistic between the HG-U133 Plus 2.0 signal values for samples in the PTCL subtype and other PTCL subtype for all samples' and (d) Correlation between the NanoString signal values and the model score for that subtype from Iqbal et al. 2014¹. Care was taken so that this curated list included both positively and negatively associated genes for each distinction. Additionally, the housekeeping genes were reduced to 16, with the lowest variance in the bridging cohort. We also added 40 other genes identified from the literature as being important to T-cell lymphoma biology. This resulted in a final 153 gene CodeSet that was used in all analyses going forward.

### Prediction of Subclassification in the Training Cohort

Multiple prediction methods, including linear discriminant analysis, lasso regression, support vector machines, and random forests, were applied to the training set in a cross-validated manner utilizing several different prediction architectures (data not shown). The method with the best cross-validated predictive accuracy was based on a series of binary linear predictors, structured as described below and illustrated in FIG. 12A and FIG. 17.

For a given pair of classes and a selected gene i, we calculate

$${w}_{i} = ( \frac{{μ}_{1 i} - {μ}_{2 i}}{{σ}_{i}^{2}} )$$

where μ₁ᵢ and μ₂ᵢ represent the mean expression values for that gene in class 1 and class 2, and σᵢ² is the pooled within-group variance estimate. Then for each sample j, we calculate a weighted score for the distinction between sets 1 and 2 as

$${S}_{12 j} = {\sum }_{i} {x}_{ij} {w}_{i} .$$

Where xij is the normalized signal value for gene i on sample j and the sum is over all genes selected for a particular distinction.

We then plotted an ROC curve based on the relationship between this score and membership in class 1 or class 2 and identified the point at which the average of sensitivity and specificity was maximized. Samples with a score above this point were identified with class 1, and those with scores below this point were identified with class 2. An exception to this rule was made for the AITL subtype classification, for which there was a region of overlap between the AITL and PTCL-NOS model scores. For samples with scores in this region it was indeterminant whether the sample was an AITL. To define this region, we considered a lower cut-point that maximized Sensitivity+2*Specificity, and an upper cut-point that maximized Specificity+2*Sensitivity. Samples above the upper cut-point were called AITL, those below the lower cut-point were called Non-AITL, those between the two cut-points were called borderline AITL.

To predict the subtype of an individual sample, we initially ran six predictors, 4 of these were trained to distinguish PTCL-NOS from cases in one of the following four categories: AITL, ALCL, ENKTCL, or ATLL. We found that the ENKTCL predictor tended to identify PTCL-NOS with cytotoxic marker expression as ENKTCL, so we additionally generated a classifier between PTCL-NOS and ENKTCL based on the viral transcript EBER. For a sample to be predicted as ENKTCL, we required that both the multigene ENKTCL and the single EBER classifier, were consistent with the molecular diagnosis. A final predictor distinguishing ALK-ALCL from ALK+ALCL was also generated. First it divided the ALCL samples according to the ALK positivity second it was used to distinguish ENKTCL samples which share some of the characteristics of ALCL but are uniformly ALK(−). If the initial set of predictors all concurred that the sample was PTCL-NOS, then a final predictor distinguishing PTCL-GATA3 from PTCL-TBX21 was applied. If more than one of the 4 predictors resulted in a non-PTCL-NOS diagnosis (e.g., both ALCL and AITL), then an additional head-to-head predictor between those types was applied to break the tie (e.g., ALCL vs. AITL), with all genes characteristic of either type selected to be included in the model.

### Pre-Analytical Assessment of FFPE RNA

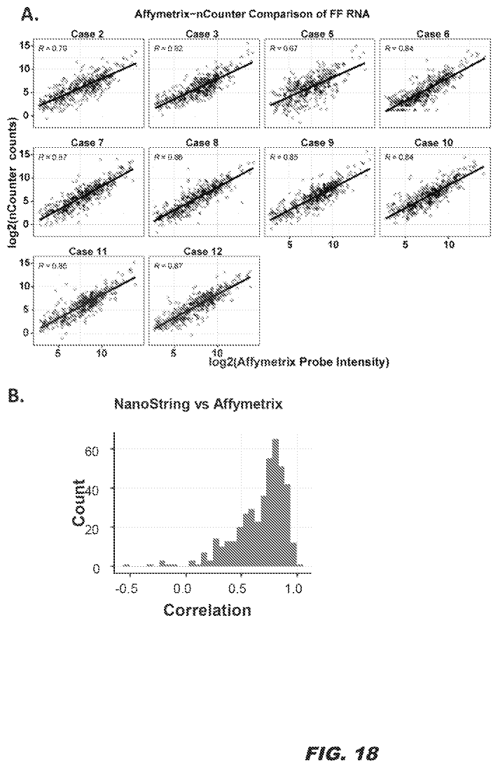

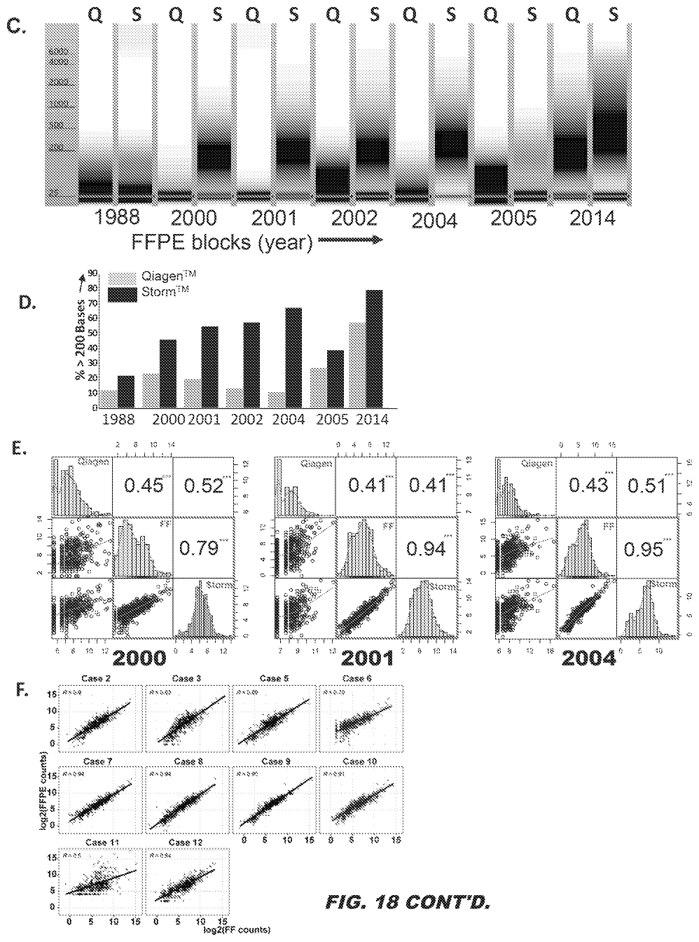

To identify transcripts that perform well on the nCounter® platform, we correlated the signal intensities for each transcript in 10 fresh frozen RNA samples profiled by both the nCounter® platform and the Affymetrix HG-U133 plus2 array. Greater than 86.5% and 65% of the transcripts showed correlations of >0.4 or >0.6 between the two platforms, respectively, and these were selected for further analysis (FIG. 18A-B). Additionally, RNA isolated from FFPE samples using the RNAstorm™ method generated longer transcripts (>200 bases) compared to another commercial kit (FIG. 18C-D) and resulted in a higher correlation coefficient with the fresh frozen RNA transcriptional profile (FIG. 18E). Thus, the RNAstorm™ isolation kit was used in the standard operating procedure for this assay for subsequent samples. We were able to isolate at least 200 ng-1 ug of total RNA per FFPE specimen with >50% of RNA showing DV200>50%. There was a high correlation between the FF RNA and corresponding FFPE RNA run on the nCounter® (NanoString, Inc) (FIG. 18F).

Reproducibility Between Sites and Adjustment Between Original Gene Set (n=442) To Refined Gene Set (n=153)

To check the reproducibility of the assay at different institutions, we selected 24 samples from the training set and reran them on the 153-gene array at two different institutions (City of Hope and Insight Genetics). Seven of these samples, along with one additional training sample, were also rerun at the original site (UNMC) on the reduced gene-set array, resulting in a total of 55 replicate samples. We observed that, for some of the predictor scores, there appeared to be a systematic bias between the models run on the 442-gene array and those run on the 153-gene array (see FIG. 20). To correct for this, we calculated the mean and standard deviation of the scores for the replicated samples on the 153-gene array (denoted by μ153 and σσ153) and on the 442-gene array (denoted by μ442 and σσ442). Using these values, we adjusted the scores produced by the 153-gene array (in particular those in the validation set) to match those of the 442-gene array according to the following formula:

$${Score}_{adjusted} = ( {Score}_{orginal} - {μ}_{153} ) \frac{{σ}_{442}}{{σ}_{153}} + {μ}_{442}$$

Note that no validation samples were used to either generate the predictive model or define this adjustment.

### Statistical Analysis

Baseline patient characteristics were compared between groups using χ2 and t-tests. The Kaplan-Meier method was used to estimate the overall survival distributions. Overall survival (OS) times were calculated as the time from diagnosis to the date of death or of last contact. Patients who were alive at last contact were treated as censored for the overall survival analysis. The differences in outcome between groups were assessed using the permutation based log rank test as implemented in the coin R-package, using the “approximation” distribution¹¹. Two-sided p<0.05 was considered significant.

### REFERENCES CITED IN THE METHODS

1. Iqbal J, Wright G, Wang C, et al. Gene expression signatures delineate biological and prognostic subgroups in 181 peripheral T-cell lymphoma. Blood. 2014; 123(19):2915-2923. 182
2. Iqbal J, Weisenburger D D, Greiner T C, et al. Molecular signatures to improve diagnosis in peripheral T-cell 183 lymphoma and prognostication in angioimmunoblastic T-cell lymphoma. Blood. 2010; 115(5):1026-1036. 184
3. Iqbal J, Weisenburger D D, Chowdhury A, et al. Natural killer cell lymphoma shares strikingly similar molecular 185 features with a group of non-hepatosplenic gammadelta T-cell lymphoma and is highly sensitive to a novel 186 aurora kinase A inhibitor in vitro. Leukemia. 2011; 25(2):348-358. 187
4. Swerdlow S H, Campo E, Harris N L, et al. WHO Classification of Tumours of Haematopoietic and Lymphoid 188 Tissues, Fourth Edition. Revised. 2017;2. 189
5. Amador C, Greiner T C, Heavican T B, et al. Reproducing the molecular subclassification of peripheral T-cell 190 lymphoma-NOS by immunohistochemistry. Blood. 2019; 134(24):2159-2170. 191
6. Akbarin M M, Shirdel A, Bari A, et al. Evaluation of the role of TAX, HBZ, and HTLV-1 proviral load on the survival 192 of ATLL patients. Blood Res. 2017; 52(2):106-111. 193
7. Li M, Green P L. Detection and quantitation of HTLV-1 and HTLV-2 mRNA species by real-time RT-PCR. J Virol 194 Methods. 2007;142(1-2):159-168. 195
8. Karmakar S, Harcourt E M, Hewings D S, et al. Organocatalytic removal of formaldehyde adducts from RNA and 196 DNA bases. Nat Chem. 2015; 7(9):752-758. 197
9. Schroeder A, Mueller O, Stocker S, et al. The RIN: an RNA integrity number for assigning integrity values to RNA 198 measurements. BMC Mol Biol. 2006; 7:3. 199
10. Wimmer I, Troscher A R, Brunner F, et al. Systematic evaluation of RNA quality, microarray data reliability and 200 pathway analysis in fresh, fresh frozen and formalin-fixed paraffin-embedded tissue samples. Sci Rep. 201 2018;8(1):6351. 202
11. Hothorn T, Hornik K, van de Wiel M A, Zeileis A. Implementing a Class of Permutation Tests: The coin Package. 203 Journal of Statistical Software. 2008; 28(8):1-23.

1. A method of differentiating between subtypes of Peripheral T-Cell Lymphoma (PTCL), the method comprising:
subjecting a sample from a subject to nucleic acid isolation;
obtaining a gene expression profile from the sample; and
identifying the subtype of PTCL based on the presence of specific genes within the gene expression profile.

2. The method of claim 1, wherein the PTCL subtype angioimmunoblastic T-cell lymphoma (AITL), anaplastic lymphoma kinase (ALK)-negative anaplastic large cell lymphoma (ALK−ALCL), adult T-cell leukemia/lymphoma (ATLL), extranodal natural killer/T-cell lymphoma (ENKTL), ALK-positive ALCL (ALK+ALCL), PTCL—not otherwise specified (PTCL-NOS), or indeterminate, wherein indeterminate indicates that the sample comprises features of at least two of the PTCL subtypes.

3. The method of claim 2, wherein:
a) the PTCL subtype is identified as AITL if the gene expression profile comprises any one or more of ACHE, ADRA2A, ARRDC4, COL4A4, EFNB2, FCAMR, GJA4, GNA14, IER3IP1, ISG15, MSR1, OLFM1, PAPLN, S1PR3, SOX8, TMEM206, TUBB2B, and VANGL2; or
b) the PTCL subtype is identified as ALK−ALCL if the gene expression profile comprises any one or more of BATF3, C9orf89, CD28, DUSP2, ICMT, LGALS1, PIK3IP1, PRKCQ, TNFRSF8, TRAT1, and ZNF101; or
c) the PTCL subtype is identified as ENKTL if the gene expression profile comprises any one or more of ATP8B4, CD3D, CD5, CTSW, FAM102A, FASLG, GZMB, KLRC2, KLRC3, KLRC4, PRF1, SH2D1B, EBER, LMP1, and TNFRSF25; or
d) the PTCL subtype is identified as ATLL if the gene expression profile comprises any one or more of ARSG, CCNEL, DOK5, FGF18, MYCN, NFATC1, NSMCEL, NUCB2, SAT1, SLC7A10, SPPL2A, STOM, TIAM2, UST, HBZ, and ZCCHC12; or
e) the PTCL subtype is identified as ALK+ALCL if the gene expression profile comprises any one or more of ALK, CACNA2D2, CCDC64, CCNA1, CCR4, DMBX1, GALNT2, GAS1, GATA3, HTRA3, ILIRAP, PCOLCE2, PFKFB3, RABGAP1L, TIAM2, TMEM158, and ZADH2; or
f) the PTCL subtype is identified as PTCL-NOS if the gene expression profile comprises any one or more of CASP2, EZH2, FLJ37453, IFI30, IFNG, LAGE3, LAP3, NR1H3, PLA2G7, SLAMF7, SLC31A2, TCN2, TMEM176B, WARS, TBX21, CXCR3, GATA3 and CCR4.

4.-8. (canceled)

9. The method of any one of claims 1-3, wherein the sample is a biopsy specimen from the subject, wherein:
a) the sample comprises formalin-fixed paraffin-embedded tissue; or
b) the sample comprises fresh frozen tissue.

10.-11. (canceled)

12. The method of any one of claims 1-3 or claim 9 further comprising providing the subject with an effective amount of a subtype-specific treatment,
wherein:
the sub-type specific treatment comprises a histone deacetylase (HDAC) inhibitor, an antifolate agent, an akylating agent, a protesome inhibitor, an antibody-drug conjugate, a phosphoinositide 3-kinases (PI3K) inhibitor, a Janus kinase (JAK) inhibitor, a signal transducer and activator of transcription (STAT) 3 inhibitor, a STAT5 inhibitor, an anaplastic lymphoma kinase (ALK) inhibitor, a hepatocyte growth factor (HGF) inhibitor, a cMET inhibitor, a platelet-derived growth factor receptor alpha (PDGFRα) inhibitor, a platelet-derived growth factor receptor beta (PDGFR) inhibitor, a mammalian target of rapamycin (mTOR) pathway inhibitor, an immune checkpoint inhibitor, a hypomethylating agent, an anti-cluster of differentiation 52 (CD52) antibody, an immunomodulatory drug, an anti-inducible T-cell co-stimulator (ICOS) antibody, a CC chemokine receptor 4 (CCR4) inhibitor, an isocitrate dehydrogenase (IDH) inhibitor, a B-cell lymphoma 2 inhibitor, an anti-interleukin-2 receptor alpha chain (CD25) antibody, a calcineurin inhibitor, a Notch signaling inhibitor, a Spleen tyrosine kinase (SYK) inhibitor, a bispecific antibody, a chimeric antigen receptor T (CAR-T) cell, or a combination thereof,
wherein:
the HDAC inhibitor comprises romidepsin, belinostat, panobinostat, or a combination thereof;
the antifolate agents comprises pralatrexate;
the akylating agent comprises bendamustine;
the proteosome inhibitor comprises bortezomib:
the antibody-drug conjugate comprises brentuximab vedotin;
the PI3K inhibitor comprises duvelisib, tenalisib, or a combination thereof;
the JAK inhibitor comprises ruxolitinib;
the ALK inhibitor comprises crizotinib;
the mTOR pathway inhibitor comprises everolimus;
the hypomethylating agent comprises 5-azacytidine;
the anti-CD52 antibody comprises alemtuzumab;
the ImiD comprises lenalidomide;
the CCR4 inhibitor comprises mogamulizumab;
the IDH inhibitor comprises enasidenib:
the BCL2 inhibitor comprises venetoclax:
the anti-CD25 antibody comprises camidanlumab tesirine;
the calcineurin inhibitor comprises cyclosporine A;
the SYK inhibitor comprises cerdulatinib;
the bispecific antibody comprises AFM13;
the chimeric antigen receptor T (CAR-T) cell comprises CD30, CD7 or both;
or a combination thereof.

13.-14. (canceled)

15. The method of claim 12, wherein:
a) the sub-type specific treatment for PTCL-NOS comprises romidepsin, belinostat, brentuximab vedotin, duvelisib, or a combination thereof; or
b) the sub-type specific treatment for AITL comprises romidepsin, 5-Aza, an isocitrate dehydrogenase (IDH) inhibitor, a calcineurin inhibitor, or a combination thereof; or
c) the sub-type specific treatment for ALK−ALCL comprises brentuximab vedotin; or
d) the sub-type specific treatment for ALK+ALCL comprises an ALK inhibitor, a platelet-derived growth factor receptor beta (PDGFR) inhibitor, or a combination thereof; or
e) the sub-type specific treatment for ATLL comprises a NOTCH inhibitor, a hepatocyte growth factor (HGF) inhibitor, a cMET inhibitor, mogamulizumab, or a combination thereof; or
f) the sub-type specific treatment for ENKTL comprises a platelet-derived growth factor receptor alpha (PDGFRα) inhibitor.

16.-20. (canceled)

21. A diagnostic kit for identifying a subtype of Peripheral T-Cell Lymphoma (PTCL) in a sample from a subject, the kit comprising at least one of a means for detecting the presence of one or a combination of genes, representing a genetic signature that is indicative of a particular PTCL subtype and instructions for use.

22. The kit of claim 21, wherein the PTCL subtype angioimmunoblastic T-cell lymphoma (AITL), anaplastic lymphoma kinase (ALK)-negative anaplastic large cell lymphoma (ALK−ALCL), adult T-cell leukemia/lymphoma (ATLL), extranodal natural killer/T-cell lymphoma (ENKTL), ALK-positive ALCL (ALK+ALCL), PTCL—not otherwise specified (PTCL-NOS), or indeterminate, wherein indeterminate indicates that the sample comprises features of at least two of the PTCL subtypes.

23. The kit of claim 22, wherein:
a) the PTCL subtype is indicative of AITL if the genetic signature comprises any one or more of ACHE, ADRA2A, ARRDC4, COL4A4, EFNB2, FCAMR, GJA4, GNA14, IER3IP1, ISG15, MSR1, OLFM1, PAPLN, S1PR3, SOX8, TMEM206, TUBB2B, and VANGL2; or
b) the PTCL subtype is indicative of as ALK−ALCL if the genetic signature comprises any one or more of BATF3, C9orf89, CD28, DUSP2, ICMT LGALS1, PIK3IP1, PRKCQ, TNFRSF8, TRAT1, and ZNF101; or
c) the PTCL subtype is indicative of ENKTL if the genetic signature comprises any one or more of ATP8B4, CD3D, CD5, CTSW, FAM102A, FASLG, GZMB, KLRC2, KLRC3, KLRC4, PRF1, SH2D1B, EBER, LMP1, and TNFRSF25; or
d) the PTCL subtype is indicative of ATLL if the genetic signature comprises any one or more of ARSG, CCNEL, DOK5, FGF18, MYCN, NFATC1, NSMCEL, NUCB2, SAT1, SLC7A10, SPPL2A, STOM, TIAM2, UST, TAX, and ZCCHC12; or
e) the PTCL subtype is indicative of ALK+ALCL if the gene signature comprises any one or more of ALK, CACNA2D2, CCDC64, CCNA1, CCR4, DMBX1, GALNT2, GAS1 GATA3, HTRA3, ILIRAP, PCOLCE2, PFKFB3, RABGAP1L, TIAM2, TMEM158, and ZADH2; or
f) the PTCL subtype is indicative of PTCL-NOS if the gene signature comprises any one or more of CASP2, EZH2, FLJ37453, IFI30, IFNG, LAGE3, LAP3, NR1H3, PLA2G7, SLAMF7, SLC31A2, TCN2, TMEM176B, and WARS.

24.-28. (canceled)

29. A method of identifying a particular subtype of Peripheral T-Cell Lymphoma (PTCL) in a sample, the method comprising:
obtaining a gene expression profile from a sample;
comparing the gene expression profile to gene signatures associated with a particular PTCL subtype, wherein each subtype of PTCL comprises a unique gene signature; and identifying the subtype of PTCL within the sample as either angioimmunoblastic T-cell lymphoma (AITL), anaplastic lymphoma kinase (ALK)-negative anaplastic large cell lymphoma (ALK−ALCL), adult T-cell leukemia/lymphoma (ATLL), extranodal natural killer/T-cell lymphoma (ENKTL), ALK-positive ALCL (ALK+ALCL), PTCL—not otherwise specified (PTCL-NOS), or indeterminate, wherein indeterminate indicates that the gene expression profile of the sample comprises genes from the unique gene signature of at least two of the PTCL subtypes.

30. The method of claim 29, wherein
the gene signature of AITL comprises any one or more of ACHE, ADRA2A, ARRDC4, COL4A4, EFNB2, FCAMR, GJA4, GNA14, IER3IP1, ISG15, MSR1, OLFM1, PAPLN, S1PR3, SOX8, TMEM206, TUBB2B, and VANGL2;
the gene signature of ALK−ALCL comprises any one or more of BATF3, C9orf89, CD28, DUSP2, ICMT, LGALS1, PIK3IP1, PRKCQ, TNFRSF8, TRAT1, and ZNF101; the gene signature of ENKTL comprises any one or more of ATP8B4, CD3D, CD5, CTSW, FAM102A, FASLG, GZMB, KLRC2, KLRC3, KLRC4, PRF1, SH2D1B, EBER, LMP1, and TNFRSF25;
the gene signature of ATLL comprises any one or more of ARSG, CCNE1, DOK5, FGF18, MYCN, NFATC1, NSMCE1, NUCB2, SAT1, SLC7A10, SPPL2A, STOM, TIAM2, UST, TAX, and ZCCHC12;
the gene signature of ALK+ALCL comprises any one or more of ALK, CACNA2D2, CCDC64, CCNA1, CCR4, DMBX1, GALNT2, GAS1, GATA3, HTRA3, ILIRAP, PCOLCE2, PFKFB3, RABGAP1L, TIAM2, TMEM158, and ZADH2;
the gene signature of PTCL-NOS comprises any one or more of CASP2, EZH2, FLJ37453, IFI30, IFNG, LAGE3, LAP3, NR1H3, PLA2G7, SLAMF7, SLC31A2, TCN2, TMEM176B, and WARS; or
a combination thereof.

31. The method of claim 29 or claim 30, wherein identifying the subtype of PTCL comprises:
a series of at least four binary predictors, wherein:
a first binary predictor comprises determining whether the gene expression profile of the sample is more consistent with the genetic signature of AITL or PTCL-NOS;
a second binary predictor comprises determining whether the gene expression profile of the sample is more consistent with the genetic signature of ALCL or PTCL-NOS;
a third binary predictor comprises determining whether the gene expression profile of the sample is more consistent with the genetic signature of ATLL or PTCL-NOS;
a fourth binary predictor comprises determining whether the gene expression profile of the sample is more consistent with the genetic signature of ENKTL or PTCL-NOS; and
assigning the PTCL subtype of the specimen based on results from the binary predictors.

32. The method of claim 31, wherein:
a) the PTCL subtype is identified as AITL when:
in the first binary predictor, the gene expression profile of the sample is more consistent with the genetic signature of AITL; and
in the second, third, and fourth binary predictors, the gene expression profile of the sample is more consistent with the genetic signature of PTCL-NOS; or
b) the PTCL subtype is identified as ALCL when:
in the second binary predictor, the gene expression profile of the sample is more consistent with the genetic signature of ALCL; and
in the first, third, and fourth binary predictors, the gene expression profile of the sample is more consistent with the genetic signature of PTCL-NOS.

33. (canceled)

34. The method of claim 32, further comprising a fifth binary predictor, wherein the fifth binary predictor comprises determining whether the gene expression profile of the sample is more consistent with the genetic signature of ALK+ALCL or ALK−ALCL; and
identifying the PTCL subtype as ALK+ALCL when the gene expression profile of the sample is more consistent with the genetic signature of ALK+ALCL; or
identifying the PTCL subtype as ALK−ALCL when the gene expression profile of the sample is more consistent with the genetic signature of ALK−ALCL.

35. The method of claim 31, wherein:
a) the PTCL subtype is identified as ATLL when:
in the third binary predictor, the gene expression profile of the sample is more consistent with the genetic signature of ATLL; and
in the first, second, and fourth binary predictors, the gene expression profile of the sample is more consistent with the genetic signature of PTCL-NOS; or
b) the PTCL subtype is identified as ENKTL when:
in the fourth binary predictor, the gene expression profile of the sample is more consistent with the genetic signature of ENKTL; and
in the first, second, and third binary predictors, the gene expression profile of the sample is more consistent with the genetic signature of PTCL-NOS, optionally wherein samples identified as ENKTL are subdivided into NK or gamma/delta T-cell lineages; or
c) the PTCL subtype is identified as PTCL-NOS when:
in the first, second, third, and fourth binary predictors, the gene expression profile of the sample is more consistent with PTCL-NOS, optionally wherein samples identified as PTCL-NOS cases are further segregated into GATA3- and TBX21-high subgroups; or
d) the PTCL subtype is identified as indeterminate when at least two of the binary predictors are not more consistent with PTCL-NOS, wherein a sample identified as indeterminate undergoes an additional binary predictor, the additional binary predictor comprising:
a first potential subtype that comprises one of the PTCL subtypes that was not more consistent with PTCL-NOS and a second potential subtype the comprises the at least one other PTCL subtype that was not more consistent with PTCL-NOS;
the method further comprising:
determining whether the gene expression profile of the sample is more consistent with the genetic signature of the first potential subtype or the second potential subtype; and
identifying the PTCL subtype as the first potential subtype when the gene expression profile of the sample is more consistent with the genetic signature of the first potential subtype; or
identifying the PTCL subtype the second potential subtype when the gene expression profile of the sample is more consistent with the genetic signature of the second potential subtype,
wherein if the PTCL subtype is identified as ALCL, the method further comprises determining the ALCL subtype as either ALK+ALCL or ALK−ALCL using the fifth binary predictor of claim 34.

36.-42. (canceled)

43. A method of treating peripheral T-cell lymphoma comprising:
obtaining a gene expression profile from a sample of a subject;
comparing the gene expression profile to gene signatures associated with a particular PTCL subtype, wherein each subtype of PTCL comprises a unique gene signature;
identifying the subtype of PTCL within the sample as either angioimmunoblastic T-cell lymphoma (AITL), anaplastic lymphoma kinase (ALK)-negative anaplastic large cell lymphoma (ALK−ALCL), adult T-cell leukemia/lymphoma (ATLL), extranodal natural killer/T-cell lymphoma (ENKTL), ALK-positive ALCL (ALK+ALCL), PTCL—not otherwise specified (PTCL-NOS), or indeterminate, wherein indeterminate indicates that the gene expression profile of the sample comprises genes from the unique gene signature of at least two of the PTCL subtypes; and
administering an effective amount of a therapeutic agent to the subject, wherein the therapeutic agent is configured to treat the identified PTCL subtype.

44. The method of claim 43, wherein the therapeutic agent comprises a histone deacetylase (HDAC) inhibitor, an antifolate agent, an akylating agent, a protesome inhibitor, an antibody-drug conjugate, a phosphoinositide 3-kinases (PI3K) inhibitor, a Janus kinase (JAK) inhibitor, a signal transducer and activator of transcription (STAT) 3 inhibitor, a STAT5 inhibitor, an anaplastic lymphoma kinase (ALK) inhibitor, a hepatocyte growth factor (HGF) inhibitor, a cMET inhibitor, a platelet-derived growth factor receptor alpha (PDGFRα) inhibitor, a platelet-derived growth factor receptor beta (PDGFRβ) inhibitor, a mammalian target of rapamycin (mTOR) pathway inhibitor, an immune checkpoint inhibitor, a hypomethylating agent, an anti-cluster of differentiation 52 (CD52) antibody, an immunomodulatory drug, an anti-inducible T-cell co-stimulator (ICOS) antibody, a CC chemokine receptor 4 (CCR4) inhibitor, an isocitrate dehydrogenase (IDH) inhibitor, a B-cell lymphoma 2 inhibitor, an anti-interleukin-2 receptor alpha chain (CD25) antibody, a calcineurin inhibitor, a Notch signaling inhibitor, a Spleen tyrosine kinase (SYK) inhibitor, a bispecific antibody, a chimeric antigen receptor T (CAR-T) cell, or a combination thereof.

45. The method of claim 44, wherein:
the HDAC inhibitor comprises romidepsin, belinostat, panobinostat, or a combination thereof;
the antifolate agents comprises pralatrexate;
the akylating agent comprises bendamustine;
the proteosome inhibitor comprises bortezomib;
the antibody-drug conjugate comprises brentuximab vedotin;
the PI3K inhibitor comprises duvelisib, tenalisib, or a combination thereof;
the JAK inhibitor comprises ruxolitinib;
the ALK inhibitor comprises crizotinib;
the mTOR pathway inhibitor comprises everolimus;
the hypomethylating agent comprises 5-azacytidine (5-Aza);
the anti-CD52 antibody comprises alemtuzumab;
the ImiD comprises lenalidomide;
the CCR4 inhibitor comprises mogamulizumab;
the IDH inhibitor comprises enasidenib;
the BCL2 inhibitor comprises venetoclax;
the anti-CD25 antibody comprises camidanlumab tesirine;
the calcineurin inhibitor comprises cyclosporine A;
the SYK inhibitor comprises cerdulatinib;
the bispecific antibody comprises AFM13;
the chimeric antigen receptor T (CAR-T) cell comprises CD30, CD7 or both;
or a combination thereof.

46. The method of claim 43, wherein:
a) the therapeutic agent comprises romidepsin, belinostat, brentuximab vedotin, duvelisib, or a combination thereof when PTCL subtype is identified as PTCL-NOS; or
b) the therapeutic agent comprises romidepsin, 5-Aza, an isocitrate dehydrogenase (IDH) inhibitor, a calcineurin inhibitor, or a combination thereof when the PTCL subtype is identified as AITL; or
c) the therapeutic agent comprises NOTCH inhibitor, a hepatocyte growth factor (HGF) inhibitor, a cMET inhibitor, mogamulizumab when the PTCL subtype is identified as ATLL; or
d) the therapeutic agent comprises brentuximab vedotin when the PTCL subtype is identified as ALK−ALCL; or
e) the therapeutic agent comprises an ALK inhibitor, a platelet-derived growth factor receptor beta (PDGFR) inhibitor, or a combination thereof when the PTCL subtype is identified as ALK+ALCL; or
f) the therapeutic agent comprises a platelet-derived growth factor receptor alpha (PDGFRα) inhibitor when the PTCL subtype is identified as ENKTL.

47.-51. (canceled)
